(19) United States
(12) Patent Application Publication
(10) Pub. No.: US 2025/0352653 A1
(43) Pub. Date: Nov. 20, 2025

(54) NEURON TARGETED 2-DEOXYGLUCOSE DENDRIMER FOR IMAGING AND TREATMENT OF NEUROLOGICAL DISEASES

(71) Applicants: Washington State University, Pullman, WA (US); University of Michigan-Dearborn, Dearborn, MI (US)

(72) Inventors: Anjali Sharma, Pullman, WA (US); Rishi Sharma, Pullman, WA (US); Anubhav Dhull, Pullman, WA (US); Aqib I. Dar, Pullman, WA (US); Zhi Zhang, Dearborn, MI (US)

(21) Appl. No.: 19/204,707

(22) Filed: May 12, 2025

Related U.S. Application Data
(60) Provisional application No. 63/647,530, filed on May 14, 2024.

(51) Int. Cl. A61K 47/60; A61K 49/00; A61P 25/00

(52) U.S. Cl. CPC A61K 47/60; A61K 49/0054; A61P 25/00; A61K 2123/00

(57) ABSTRACT

Provided herein is a dendrimer complex comprising a 2-deoxyglucose (2DG) dendrimer and a neuroactive agent conjugated to an outer surface of the dendrimer. The dendrimer complex may further include one or more imaging agents and/or radioligands conjugated to an outer surface of the dendrimer. Such complexes are useful in methods for detecting or treating neurological diseases or disorders.

### CROSS-REFERENCE TO RELATED APPLICATIONS

This application claims benefit of U.S. provisional patent application 63/647,530 filed May 14, 2024, the contents of which are incorporated herein by reference.

### FIELD OF THE INVENTION

The invention is generally related to a 2-deoxyglucose dendrimer useful for imaging and treatment of neurological diseases. In particular, the dendrimer provides for targeted delivery of drugs to neurons at the site of injury in the brain.

### BACKGROUND OF THE INVENTION

Delivering therapeutic molecules across the blood-brain barrier (BBB) has long been the most prominent obstacle in the treatment of brain disorders. Roughly 98% of drugs identified through high-throughput screening fail to advance to the next phase of drug development because they cannot effectively penetrate this protective barrier. Due to this reason, there is a greater incidence of failures during the later stages of CNS drug development as compared to non-CNS drugs. Lately, nanomaterials have become indispensable in the diagnosis and targeted treatment of various unmet clinical needs including brain diseases. Nanoparticles not only improve drug pharmacokinetics and biodistribution, but also offer controlled release kinetics at the intended target site by navigating through various biological barriers effectively. Efforts have persistently been underway to develop novel nanocarriers which are capable of precisely transporting drugs across the BBB to target the specific regions of brain damage. However, there is a limited presence of nanocarriers in the literature which are capable of specifically delivering therapies to neurons at the site of brain injury from non-invasive systemic administration routes. Even if drugs or nanoparticles get across the impaired BBB following brain injury or neuroinflammation, their uptake into the critical brain cells such as neurons, involved in brain diseases remains challenging. Targeting neurons is specifically more complex since they are far lower in number and less phagocytic in nature compared to the other immune cells in the brain. Moreover, the brain comprises various neuronal subtypes, each serving distinct functions, making it crucial to pinpoint those relevant to the specific disease.

Traumatic brain injury (TBI) stands as a prominent global contributor to fatalities and impacts countless individuals, with even more dire consequences in low and middle-income countries. Survivors endure long term disabilities, compromised neurological function, shifts in behavior, depression and require extensive long-term rehabilitation. The pathology of TBI is complicated and involves a primary insult due to direct physical trauma to the brain, which in turn leads to a secondary insult, such as neuroinflammation, oxidative stress and excitotoxicity, caused by destructive biochemical cascades ultimately leading to the death of glia and neurons. Microglia and astrocytes are activated after encountering TBI, which leads to the overproduction of neuroinflammatory mediators that intensify TBI, resulting in neuronal damage. Although huge research advancements have been made in the field of TBI, there is no approved therapy available to mitigate long term outcomes. Numerous potential treatments have faced challenges in late-stage clinical trials because they didn't achieve the necessary drug levels at the specific disease site. These drug delivery obstacles can be surmounted by developing innovative and biocompatible nanocarriers that possess enhanced targeting capabilities. Of particular significance is the precise delivery of drugs to vital cells like neurons at the site of brain injury.

Within the realm of polymer-based nanoparticulate drug delivery systems, dendrimers, which are hyper-branched, uniformly sized, monodispersed synthetic macromolecules with precisely defined structure and composition, have gained extensive utilization in the field of drug delivery systems. This arises from the meticulous control over their properties like molecular structure, size, shape and solubility. Dendrimers also offer the prospect of attaching targeting agents, imaging dyes, small molecule therapeutics and biologics to their multi-valence surface groups, enabling precise targeting, imaging, and therapeutic interventions for various diseases. Notwithstanding these benefits, the successful translation of different dendrimer-based drug delivery systems to clinical applications remains infrequent. Beyond the obstacle of attaining precise target specificity, there are additional challenges in the clinical implementation and commercialization of dendrimer-based drug delivery, including concerns related to cytotoxicity, scalability, structural imperfections, complex synthetic design, consistency in production, product purity, and in vivo stability. Novel platforms for targeted delivery of drugs to neurons for the treatment of neurological diseases/disorders are needed.

### SUMMARY

Disclosed herein is the rational design and synthesis of a 2-deoxy glucose (2DG) surfaced dendrimer (2DG-D) for targeted delivery of drugs to neurons at the site of injury in the brain. 2DG is incorporated at the surface of 2DG-D to achieve neuron targeting via GLUT transporters. To avoid any delivery of 2-deoxyglucose to neurons in injured brain regions which in some neurological conditions can have deleterious effects, robust chemical linkages may be utilized within the dendrimer backbone, preventing degradation, and enabling efficient clearance from non-target organs, primarily through renal excretion.

An aspect of the disclosure provides a dendrimer complex comprising a 2-deoxyglucose (2DG) dendrimer; and a neuroactive agent conjugated to an outer surface of the dendrimer, wherein the 2DG dendrimer is not conjugated to a prostate-specific membrane antigen (PSMA) ligand. In some embodiments, the dendrimer is a generation 0-10 dendrimer. In some embodiments, the dendrimer is a mixed layer dendrimer. In some embodiments, the neuroactive agent is pioglitazone or rosiglitazone. In some embodiments, the complex further comprises a mitochondria targeting moiety conjugated to an outer surface of the dendrimer. In some embodiments, the mitochondria targeting moiety is selected from the group consisting of triphenylphosphonium (TPP), rhodamine derivatives, dequalinium (DQA), peptide-based targeting ligands, tetraphenylethylene (TPE) based molecule, mitochondria-penetrating peptides, indolinium based compounds, and szeto-schiller (SS) peptides. In some embodiments, the complex further comprises one or more imaging agents and/or radioligands conjugated to an outer surface of the dendrimer.

Another aspect of the disclosure provides a pharmaceutical composition comprising a dendrimer complex as described herein and a pharmaceutically acceptable carrier.

Another aspect of the disclosure provides a method of delivering a neuroactive agent across the blood-brain barrier in a subject in need thereof, comprising administering to the subject a therapeutically effective amount of a dendrimer complex as described herein. In some embodiments, the dendrimer complex is administered via systemic administration. In some embodiments, the subject has a traumatic brain injury.

Another aspect of the disclosure provides a method of imaging neurons, comprising contacting the neurons with a dendrimer complex as described herein, wherein the dendrimer complex further comprises an imaging agent conjugated to an outer surface of the dendrimer; and detecting the imaging agent.

### BRIEF DESCRIPTION OF THE DRAWINGS

FIGS. 1A-C. Synthetic route to 2DG-dendrimer (2DG-D). A) Synthetic route to a clickable dendron containing three 2DG molecules; B) Synthesis of 2DG-D from 8-arm alkyne core; C) structure of 2DG-D depicting 24-2DG molecules at the surface.

FIGS. 2A-C. Characterization of 2DG-dendrimer. A) ¹H NMR spectra of intermediate dendron, hyper-core, protected and deprotected 2DG-D representing the appearance and disappearance of characteristic protons; B) HPLC chromatogram of 2DG-D showing>99% purity; C) Table representing the physicochemical properties of 2DG-D.

FIGS. 3A-C. Reproducible synthesis of 2DG-D and Cy5 conjugation. A) The figure shows the reproducibility in the synthesis of multiple 5 g-scale batches of 2DG-D via ¹H NMR and HPLC. The ¹H NMR spectra of 3 batches (5 g scale) show characteristic protons from 2DG-D. The HPLC chromatographs show reproducibility in the purity of these batches with all these batches showing>99% purity. B) Conjugation of near-infrared dye Cy5 on 2DG-D for imaging and biodistribution studies. C) HPLC chromatogram showing a shift in retention time during 2DG-D-Cy5 synthesis. The 2DG-D-Cy5 is stable when incubated in plasma at physiological temperature for 24 h.

FIGS. 4A-B. Brain and organ biodistribution of 2DG-D in a pediatric mouse model of TBI. A) Quantitative biodistribution of 2DG-D-Cy5 in TBI animals in injured and non-injured regions at different time points (1, 4, and 24 h; n=6-7) as compared to age-matched sham controls (n=6). A significant increase in the dendrimer uptake was detected in the injured brain of TBI animals as compared to non-injured brain of TBI animals and to the brains of sham animals (**** P<0.0001; *** P<0.001; ** P<0.01). C) Quantitative biodistribution of 2DG-D-Cy5 in the major organs and serum of TBI animals at different time points (1, 4, and 24 h; n=6-7). The data was obtained through fluorescence spectroscopy of homogenized tissue extracts containing 2DG-D-Cy5 and reported as a percentage of the injected dose in total organ (or total serum volume).

FIGS. 5A-F. Synthesis and characterization of 2DG-D-pioglitazone (2DG-D-Pio) conjugate. A) Attachment of clickable linker on Pio; B) Conjugation of Pio-azide on 2DG-D to synthesize 2DG-D-Pio conjugate; C) 2DG-D-Pio demonstrates several folds higher water solubility than free Pio; D) ¹H NMR spectrum of 2DG-D-Pio depicting characteristic drug and dendrimer protons; E) 2DG-D-Pio demonstrates>99 purity via HPLC; F) 2DG-D-Pio shows sustained drug release profile at intracellular conditions.

FIGS. 6A-F. Evaluation of behavioral outcomes. Male and female mice littermates from the same litter were randomly divided into sham (n=14, 8M/6F), TBI+saline (n=14, 7M/7F), TBI+Pio (n=19, 10M/9F), and TBI+2DG-D-Pio (n=23, 12M/11F) groups. Mice in the TBI groups received free Pio (5 mg/kg, 100 μL), 2DG-D-Pio (containing 5 mg/kg pioglitazone, 100 μL) or PBS (100 μL) at 6-h post-injury. Sham group did not receive any treatment. Behavioral testing were performed at 24-h post-treatment. Data were presented as mean+SEM. Treatment groups were presented as sham, TBI+saline, TBI+Pio, and TBI+2DG-D-Pio. A) The body weight gain significantly decreased in both male TBI+saline and TBI+Pio groups, compared with the male sham group, and in the male TBI+Pio group, compared with male TBI+2DG-D-Pio group. B) The grip strength significantly decreased in both male and female TBI+saline and TBI+Pio groups, compared with the male and female sham and TBI+2DG-D-Pio groups. C) In the Rotarod test, the latency to the first fall significantly decreased in both male and female TBI+saline and TBI+Pio groups, compared with the male and female sham groups. In addition, the latency to the first fall significantly decreased in the male TBI+saline (p<0.05) and TBI+Pio groups, compared with the male TBI+2DG-D-Pio group. D) The immobile time significantly increased in both male and female TBI+saline and TBI+Pio groups, compared with the male and female sham and TBI+2DG-D-Pio groups. E) The time spent in the light chamber significantly increased in the female TBI+Pio group, compared with the female sham group. F) The time spent with the novel object significantly decreased in the male TBI+saline group, compared with the male sham and TBI+2DG-D-Pio groups. The time spent with the novel object significantly decreased in the female TBI+saline group, compared with the female TBI+2DG-D-Pio group *, p<0.05; **, p<0.01; n.s, no significance.

FIGS. 7A-H. mRNA expressions of pro- and anti-inflammatory markers and cell death markers from sham (n=8, 4M/4F), TBI+saline (n=10, 5M/5F), TBI+Pio (n=10, 5M/5F), and TBI+2DG-D-Pio (n=10, 5M/5F) groups. Neurons were isolated from the injured brain regions (or the matching area from the sham mice) at 24-h post-treatment for gene expression evaluations. Data were presented as mean+SEM. Treatment groups were presented as sham, TBI+saline, TBI+Pio, and TBI+2DG-D-Pio. A-D) The expression of pro-inflammatory makers, TNF-α (A), IL-1β (B), TLR4 (C), and NLRP3 (D). E-F) The expression of anti-inflammatory makers IL-10 (E) and IL-13 (F). G-H) The expression of cell death makers caspase-3 (G) and Fas (H). *, p<0.05; **, p<0.01; ***, p<0.001; n.s., no significance.

FIGS. 8A-B. A) Synthesis of Rosi intermediates and 2DG-D-Rosi. Reagents and conditions: (i) TEA, Anhy. DMF, RT, 10 hours; (ii) EDC.HCl, DMAP, Anhy. DMF, RT, 4 hours; (iii) EDC.HCl, DMAP, dry DMF, RT, 24 hours; (iv) CuBr, PMDETA, THPTA, Anhy. DMF, RT, 10 hours; B) ¹H NMR characterization of Rosiglitazone intermediates and 2DG-D-Rosi showing characteristic protons signals.

FIGS. 9A-E. A) HPLC traces of Rosi intermediates, 2DG-D, 2DG-D-Hexyne, and 2DG-D-Rosi, confirming successful conjugation. B) A significant enhancement in the water solubility of Rosi upon conjugation with 2DG-D. C) Physicochemical properties of 2DG-D-Rosi. D) Size distribution and zeta potential of 2DG-D-Rosi. E) In vitro drug release study of 2DG-D-Rosi over 20 days, demonstrating sustained release of Rosi.

FIGS. 10A-E. Evaluation of biocompatibility and neuronal uptake of dendrimers.

A-D) Cytocompatibility assay of 2DG-D-Rosi conjugates at different concentrations with (A) CATH.a and (B) HUVEC, and (C) EOC 20 cells, and (D) hemocompatibility index of 2DG-D-Rosi dendrimer conjugates. E) Confocal microscopy based quantitative uptake of 2DG-D-Rosi dendrimers in CATH.a cells in the presence of trafficking inhibitors. For cytocompatibility and hemocompatibility studies, the statistical significance was calculated using ordinary one-way ANOVA, ****, p<0.0001; ns-nonsignificant.

FIGS. 11A-F. Effect of 2DG-D-Rosi on regulating inflammatory response and mitochondrial health. Change in A) TNF-α, B) IL-6 and C) NO levels after Rosi or 2DG-D-Rosi treatment in LPS: H2O2 Induced CATH.a Cells. D) Quantitative ImageJ® bar gaph dipcting MFI of j-monomer/j-aggregate in Rosi or 2DG-D-Rosi treated CATH.a cells. E) Quantitative analysis showing percentage cells with polarized and depolarized mitochondria. F) dot plots showing j-monomer/j-aggregate stained cell populations. The scale bar is 250 μm and the statistical significance was calculated using one-way ANOVA, ****, p<0.0001; ***, p<0.001; **, p<0.01; *, p<0.05; ns-nonsignificant.

FIGS. 12A-B. 2DG-D-Rosi attenuates apoptosis and necrosis in CATH.a cells. A) Apoptosis/necrosis analysis performed via flow cytometry in treated CATH.a neuronal cell. The dot plots show Annexin V (Alexa Fluor® 488, YL-1) and Propidium Iodide (PI, BL-1) staining. The quadrants indicate live cells (Annexin V-/PI-), early apoptotic cells (Annexin V+/PI-), late apoptotic/necrotic cells (Annexin V+/PI+), and necrotic cells (Annexin V-/PI+). B) Quantitative analysis showing percentage of cells in apoptotic/necrotic stage. For apoptosis/necrosis assay, the p-values were calculated between the blank CATH.a cells and the treatments, as well as between Rosi and 2DG-D-Rosi using ordinary one-way/two-way ANOVA with, ***, p<0.001; *, p<0.05; ns-nonsignificant.

FIGS. 13A-B. Preparation and structural elucidation of 2DG-D-TPP-Cy5 conjugate. (A) Schematic representation for the synthesis of 2DG-D-TPP-Cy5 using CuAAC reaction. Reagents and conditions: (i) DMF, HOBt, DIPEA, HBTU, RT, 12 hours, 77% (ii) DMF, EDC.HCl, DMAP, RT, 24 hours, 80% (iii) CuSO₄.5H₂O, Sodium ascorbate, DMF, DI Water, 40° C., 10 hours, 86% (iv) CuSO₄.5H₂O, Sodium ascorbate, DMF, DI Water, 40° C., 10 hours, 83% (B) ¹H NMR spectra showing characteristic proton signals of 2DG-D, 2DG-D-Hexyne, TPP-Acid, TPP-Azide, 2DG-D-TPP and 2DG-D-TPP-Cy5 at each step of the synthesis.

FIG. 14. Characterization of 2DG-D-TPP-Cy5 conjugate and intermediates: HPLC chromatogram showing shift in the retention time at different steps of synthesis and percentage purity of 2DG-D-TPP-Cy5.

FIGS. 15A-B. Preparation and structural elucidation of 2DG-D-TPP-Pio conjugate. (A) Schematic representation for the synthesis of 2DG-D-TPP-Pio using CuAAC reaction. Reagents and conditions: (i) DMF, EDC.HCl, DMAP, RT, 24 hours, 80% (ii) CuSO₄.5H₂O, Sodium ascorbate, DMF, DI Water, 40° C., 10 hours, 85% (iv) CuSO₄.5H₂O, Sodium ascorbate, DMF, DI Water, RT, 12 hours, 88% (B) ¹H NMR spectra showing characteristic proton signals of 2DG-D-Hexyne′, 2DG-D-TPP′, Pio-Azide and 2DG-D-TPP-Pio at each step of the synthesis.

FIG. 16. 2DG-D-TTP-Pio-Cy5 was co-localized with the mitochondria of the neurons at the site of injury. The arrows pointed out some of the co-localization of mitochondria (Mito), neurons (NeuN) and D-TTP-Cy5. Scale bars: 50 μm.

FIGS. 17A-F. Evaluation of behavioral outcomes. Data were presented as mean ±SEM. Treatment groups were presented as sham (circles), TBI+vehicle (triangles), TBI+Pio (upside-down triangles), TBI+D-Pio (diamond) and TBI+D-TTP-Pio (hexagon). A) The changes in the body weight at 24-h post-treatment. B) The grip strength test. C) The change in the latency to the first fall in the Rotarod test. D) The immobile time in the tail suspension test. E) The time spent in the light chamber during the light/dark box test. F) The time spent with the novel object during the novel object recognition test. *, p<0.05; **, p<0.01; ***, p<0.001; ****, p<0.0001.

FIGS. 18A-F. mRNA expressions of Creb1, Catalase, Cycs, Timm23, Aif and Pparg from sham (circles), TBI+vehicle (triangles), TBI+Pio (upside-down triangles), TBI+D-Pio (diamond) and TBI+D-TTP-Pio (hexagon) groups. Data were presented as mean±SEM. The mRNA expression of Creb1 (A), Catalase (B), Cycs (C), Timm23 (D), Aif (E) and Pparg (F) in both male and female treatment groups. *, p<0.05; **, p<0.01; ***, p<0.001′ ****, p<0.0001.

### DETAILED DESCRIPTION

Embodiments of the disclosure provide compositions and methods for delivering drugs across the blood-brain barrier using a 2-deoxy glucose (2DG) surfaced dendrimer to achieve neuron targeting via GLUT transporters.

A dendrimer is a synthetic highly branched monodisperse and polyfunctional macromolecule, constituted by repetitive units (so-called “generations”) that are chemically bound to each other by an arborescent process around a multifunctional central core. Dendrimers can be considered to have three major portions: a core, an inner shell, and an outer shell. Exemplary chemical moieties for the core, inner shell, and outer shell are independently selected from dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3″-silanetetrayltetrakis(propane-1-thiol), 3,3-divinylpenta-1,4-diene, 3,3′,3″-nitrilotripropionic acid, 3,3′,3″-nitrilotris(N-(2-aminoethyl) propanamide), 3,3′,3″,3′ “-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy)) bis(ethan-1-ol), tetrakis(3-(trichlorosilyl) propyl) silane, 1-Thioglycerol, 2,2,4,4,6,6-hexachloro-1,3,5,215,415,615-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl)benzene-1,2,3-triol, 5-(hydroxymethyl)benzene-1,3-diol, 1,3,5-tris(dimethyl (vinyl) silyl)benzene, Carbosiloxane core, nitrilotrimethanol, ethylene diamine, propane-1,3-diamine, butane-1,4-diamine, 2,2′,2″-nitrilotris(ethan-1-ol), alpha cyclodextrin, beta cyclodextrin, gamma cyclodextrin, benzene-1,2,3,4,5,6-hexathiol, monosaccharide, disaccharides, trisaccharides, oligosaccharides, chitosan, and derivatives thereof.

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures. Generally, dendrimers have a diameter from about 1 nm up to about 50 nm, such as from about 1-5, 5-10, 10-15, 15-20, 20-25, 25-30, 35-40, 40-45 or 45-50 nm in diameter, including all single digits within these ranges.

Applications of dendrimers typically involve conjugating other chemical species to the dendrimer surface that can function as detecting agents (such as a dye molecule), affinity ligands, targeting components, radioligands, imaging agents, or pharmaceutically active compounds. As described herein, 2-deoxy glucose (2DG) surfaced dendrimers conjugated to a neuroactive agent may be used as a nanoplatform to target neurons thus enhancing drug effectiveness while mitigating dose-related toxicity and systemic side effects.

Drug attachment to the dendrimer may be accomplished by (1) a covalent attachment or conjugation to the external surface of the dendrimer forming a dendrimer prodrug, (2) ionic coordination to charged outer functional groups, or (3) micelle-like encapsulation of a drug via a dendrimer-drug supramolecular assembly.

Dendrimers are also classified by generation, which refers to the number of repeated branching cycles that are performed during its synthesis. For example, if a dendrimer is made by convergent synthesis, and the branching reactions are performed onto the core molecule three times, the resulting dendrimer is considered a third generation dendrimer. Each successive generation results in a dendrimer roughly twice the molecular weight of the previous generation. Dendrimers may have a single surface functional group, or may be modified to allow for multiple functional groups on the surface.

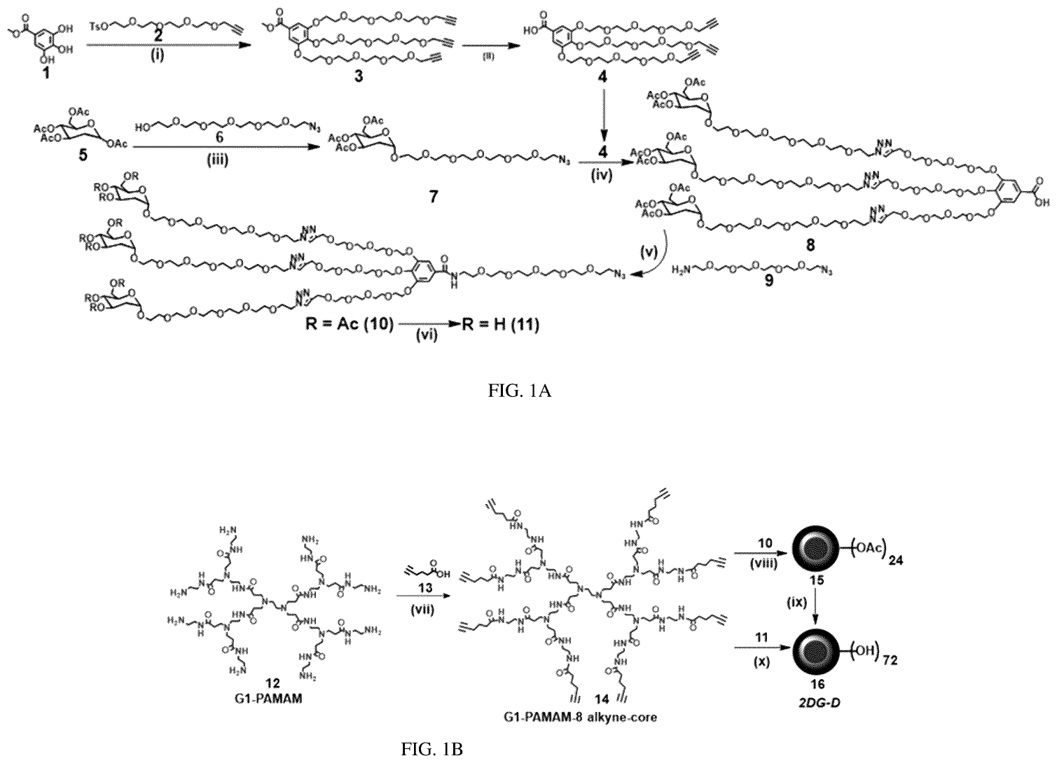

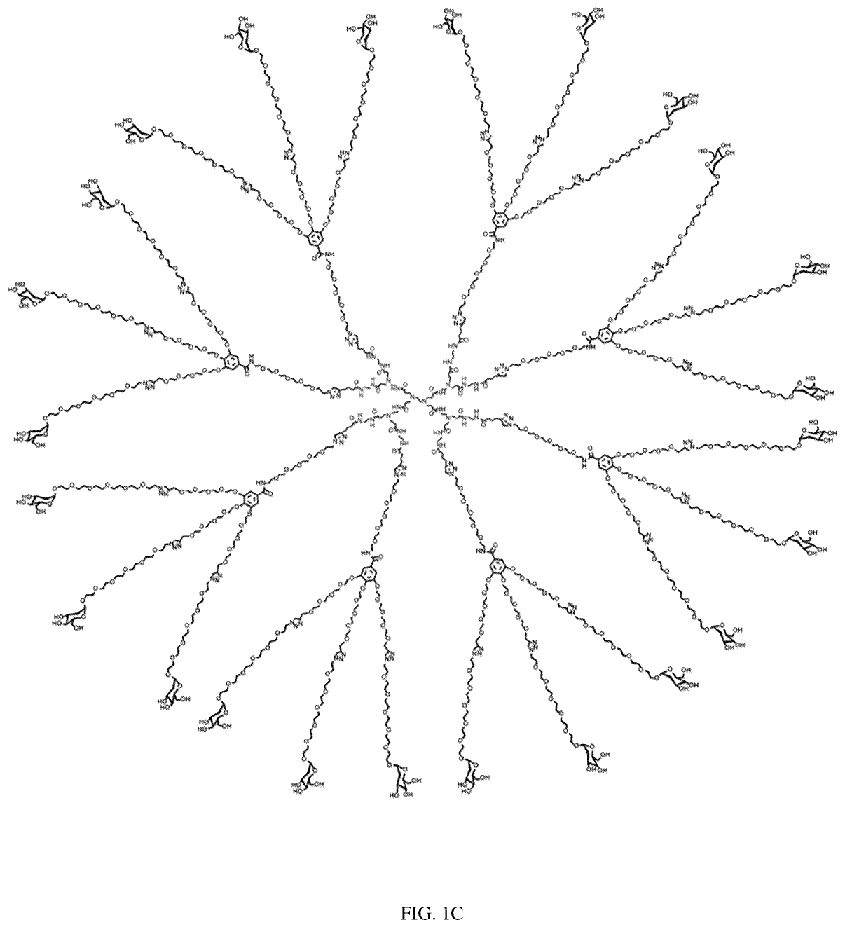

Suitable dendrimers may be generation 0 to generation 10. In preferred embodiments, the dendrimer is a generation 2, 3, 4, 5 or 6 dendrimer. Dendrimers may be composed of mixed layers or identical layers. Suitable dendrimer layers include phosphorous dendrimers, peptide dendrimers, polyamidoamine (PAMAM) dendrimers, polypropyleneimine (PPI) dendrimers, polyethyleneimine (PEI) dendrimers, polyethylene glycol-based dendrimers, polyester dendrimers, polylysine dendrimers, polypropylamine (POPAM) dendrimers, iptycene dendrimers, aliphatic poly(ether) dendrimers, aromatic polyether dendrimers, micellar dendrimers, and glycodendrimers. A glycodendrimer may encompass (1) carbohydrate-coated; (2) carbohydrate-centered; or (3) carbohydrate-based dendrimers. For example, a 2DG dendrimer may include an innermost layer (the core) comprising alkyne-terminating generation-1 PAMAM dendrimer or small molecule based cores, followed by a second layer composed of gallic acid building blocks, and the outermost layer comprising 2-DG (FIG. 1C).

In certain embodiments, all the branching units present in the dendrimer are 2-deoxyglucose-surfaced branching units. In certain embodiments, the central core comprises polyamidoamine, cyclodextrin, polylysine, 2,2-bismethylolpropionic, tetrazine, poly(propylene imine), polyethylene glycol, glycol, or adamantane, or molecules with multiple hydroxyl, carboxylic acid, alkynes, azides, amines, strained alkynes, or tetrazine functional groups. In certain embodiments, the branching units include at least one focal point selected from triazole, gallic acid, an amino acid, a peptide, or a linear polymer. In certain embodiments, the branching units include repeating units selected from polyethylene glycol, an alkane, a peptide, an amino acid, or a linear polymer. In certain embodiments, the dendrimer includes 1 to 200 2-deoxyglucose-surfaced branching units.

In certain embodiments, the dendrimer can include a plurality of linkers on the core, each linker having from 1 to 50 hydrocarbon units. In certain embodiments, the dendrimers can include a plurality of linkages in the dendrimer molecule backbone selected from disulfide, ester, ether, carbonate, carbamate, thiol, thioester, cathepsin sensitive, maleimidomethyl, thioether, hydrazine, glucuronide bond, hydrazides, N-alkyl, ethyl, hydroxymethyl, and amide.

The molecular weight of the dendrimers can be varied to prepare polymeric nanoparticles that form particles having properties, such as drug release rate, optimized for specific applications. The dendrimers can have a molecular weight of between about 150 Da and 1 MDa. In certain embodiments, the polymer has a molecular weight of between about 500 Da and about 100 kDa, more preferably between about 1 kDa and about 50 kDa, most preferably between about 1 kDa and about 20 kDa.

Dendrimers may have a certain surface density of particular functional groups (e.g. hydroxyl groups). For example, the dendrimer may have a surface density of a certain functional group of at least 1 functional group/nm2 (number of surface groups/surface area in nm2). For example, in some embodiments, the surface density of certain functional groups is between about 1 and about 50, e.g. more than 2, 3, 4, 5, 6, 7, 8, 9, 10, 15, 20, 25, 30, 35, 40, 45, 50, or more than 50 group/nm2.

In some embodiments, the dendrimers may have a fraction of the preferred functional groups exposed on the outer surface, with the others in the interior core of the dendrimers. For example, the dendrimers have a volumetric density of certain functional groups of at least 1 functional group/nm³ (number of functional groups/volume in nm³). For example, in some embodiments, the volumetric density of functional groups is between about 1 and about 50, e.g. more than 2, 3, 4, 5, 6, 7, 8, 9, 10, 15, 20, 25, 30, 35, 40, 45, 50, or more than 50 group/nm³.

Dendrimers may be prepared by methods known in the art. Dendritic structures are mostly synthesized by two different approaches: divergent or convergent. Many other synthetic pathways exist, such as the orthogonal approach, accelerated approaches, the double-stage convergent method or the hypercore approach, the hypermonomer method or the branched monomer approach, the double exponential method; the orthogonal coupling method or the two-step approach, the two monomers approach, or the AB₂-CD₂ approach.

The core of the dendrimer, one or more branching units, one or more linkers/spacers, and/or one or more surface groups can be modified to allow conjugation to further functional groups (branching units, linkers/spacers, surface groups, etc.), monomers, targeting agents, and/or active agents via click chemistry. “Click chemistry” involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition) (or equivalent thereof) (or any active end group such as, for example, a primary amine end group, a hydroxyl end group, a carboxylic acid end group, a thiol end group, etc.) on the second moiety.

A neuroactive agent is conjugated to the 2DG dendrimer as described herein. A neuroactive agent is any agent that influences the activity of the nervous system, particularly the brain. These agents act on neurons, affecting how they transmit signals and interact with each other. Exemplary agents include proteins or peptides, sugars or carbohydrate, nucleic acids or oligonucleotides, lipids, small molecules, or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the active agent is a therapeutic antibody. One or more types of active agents can be encapsulated, complexed or conjugated to the dendrimer. In preferred embodiments, exemplary neuroactive agents include, but are not limited to Pioglitazone, glutathione, N-acetyl cysteine, lipoic acid, ebselen, minoycline, disulfram, Resveratrol, vitamin E, Melatonin, Coenzyme Q 10 (CoQ10), Vitamin C & E, Lactoferrin, Gallic acid, Galantamine, Celecoxib, dexamethasone, curcumin, Dopamine, and rosmarinic acid.

The dendrimer complexes described herein may be used to deliver any poorly water-soluble neuroactive agent. As used herein, the term “poorly water-soluble” or “lipophilic” refers to having a solubility in water at 20° C. of less than 1%, e.g., 0.01% (w/v), i.e., a “sparingly soluble to very slightly soluble drug” as described in Remington, The Science and Practice of Pharmacy, 19th Edition, A. R. Gennaro, Ed., Mack Publishing Company, Vol. 1, p. 195 (1995).

A number of drugs have been developed and used in an attempt to interrupt, influence, or temporarily halt the glutamate excitotoxic cascade toward neuronal injury. One strategy is the “upstream” attempt to decrease glutamate release. This category of drugs includes riluzole, lamotrigine, and lifariLine, which are sodium channel blockers. The commonly used nimodipine is a voltage-dependent channel (L-type) blocker. Attempts have also been made to affect the various sites of the coupled glutamate receptor itself. Some of these drugs include felbamate, ifenprodil, magnesium, memantine, and nitroglycerin. These “downstream” drugs attempt to influence such intracellular events as free radical formation, nitric oxide formation, proteolysis, endonuclease activity, and ICE-like protease formation (an important component in the process leading to programmed cell death, or apoptosis).

Neuroactive agents for the treatment of neurodegenerative diseases are well known in the art and can vary based on the symptoms and disease to be treated. For example, conventional treatment for Parkinson's disease can include levodopa (usually combined with a dopa decarboxylase inhibitor or COMT inhibitor), a dopamine agonist, or an MAO-B inhibitor. Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines.

Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

Riluzole (RILUTEKO) (2-amino-6-(trifluoromethoxy)benzothiazole), an antiexcitotoxin, has yielded improved survival time in subjects with ALS. Other medications, most used off-label, and interventions can reduce symptoms due to ALS. Some treatments improve quality of life and a few appear to extend life. Common ALS-related therapies are reviewed in Gordon, Aging and Disease, 4 (5): 295-310 (2013), see, e.g., Table 1 therein. Additional therapies may include an agent that reduces excitotoxicity such as talampanel (8-methyl-7H-1,3-dioxolo (2,3)benzodiazepine), a cephalosporin such as ceftriaxone, or memantine; an agent that reduces oxidative stress such as coenzyme Q10, manganoporphyrins, KNS-760704R6R)-4,5,6,7-tetrahydro-N6-propyl-2,6-benzothiazole-diaminedihydrochloride, RPPX], or edaravone (3-methyl-1-phenyl-2-pyrazol in-5-one, MCI-186); an agent that reduces apoptosis such as histone deacetylase (HDAC) inhibitors including valproic acid, TCH346 (Dibenzo (b,f) oxepin-10-ylmethyl-methylprop-2-ynylamine), minocycline, or tauroursodeoxycholic Acid (TUDCA); an agent that reduces neuroinflammation such as thalidomide and celastol; a neurotropic agent such as insulin-like growth factor 1 (IGF-1) or vascular endothelial growth factor (VEGF); a heat shock protein inducer such as arimoclomol; or an autophagy inducer such as rapamycin or lithium.

Treatment for Alzheimer's Disease can include, for example, an acetylcholinesterase inhibitor such as tacrine, rivastigmine, galantamine or donepezil; an NMDA receptor antagonist such as memantine; or an antipsychotic drug. Treatment for Dementia with Lewy Bodies can include, for example, acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48 (1): 1-8 (2012)).

Exemplary neuroactive agents that act as a neuroprotective agent are also known in the art and include, for example, thiazolidinediones, glitazones, glutamate antagonists, antioxidants, and NMDA receptor stimulants. Other neuroprotective agents and treatments include caspase inhibitors, trophic factors, anti-protein aggregation agents, therapeutic

Other common neuroactive agents for treating neurological dysfunction include amantadine and anticholinergics for treating motor symptoms, clozapine for treating psychosis, cholinesterase inhibitors for treating dementia, and modafinil for treating daytime sleepiness.

The neuroactive agent may be an agent (e.g. chemotherapeutic agent) useful for treating a brain tumor. The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The types of cancer that can be treated with the compositions and methods include, but are not limited to, brain tumors including glioma, glioblastoma, gliosarcoma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, ganglioma, Schwannoma, cordomas and pituitary tumors.

In some embodiments, the dendrimer is conjugated to a mitochondria targeting ligand to further help the 2DG dendrimer which is being taken up by injured neurons to be taken up by the mitochondria of those neurons. Triphenylphosphonium, for example, is an effective targeting ligand for mitochondria. Triphenylphosphine, with its overall cationic charge, exploits the negative membrane potential of mitochondria, thereby facilitating precise targeting. Additional exemplary mitochondria targeting moieties include, but are not limited to, rhodamine derivatives, dequalinium (DQA), peptide-based targeting ligands, tetraphenylethylene (TPE) based molecule, mitochondria-penetrating peptides, indolinium based compounds, and szeto-schiller (SS) peptides. The dendrimer may be conjugated to the mitochondrial targeting ligand with a releasable or non-releasable covalent bond. The dendrimer described herein is not conjugated to a prostate specific membrane antigen (PSMA) ligand.

The agents/ligands described herein are “conjugated” to a functional group on the outer surface of the dendrimer, generally a group that is surface exposed and available to react with the agent/ligand. “Conjugated” as used herein refers to covalently bonded to a surface functional group of the dendrimer.

The dendrimers employed herein generally exhibit a surface charge that is neutral (uncharged). That is to say, the functional groups on the surface of the dendrimer, e.g. hydroxyl, acetyl, or other polar uncharged groups, provide a neutral (not cationic or anionic) surface charge. In general, the dendrimer has a zeta potential value that is close to zero, e.g. within a range of-10 mV to +10 mV.

Generally, the neuroactive agent (or other additional agent/ligand such as the mitochondrial targeting ligand) is conjugated to an outer surface of the dendrimer at a concentration of about 0.01% to about 30%, preferably about 1% to about 20%, more preferably about 5% to about 15% by weight. In some embodiments, the neuroactive agent (or other additional agent/ligand such as the mitochondrial targeting ligand) is conjugated to about 1-30% of the surface functional groups (e.g. hydroxyl groups).

In some embodiments, one or more agents, such as one or more drugs, imaging agents, and/or radioligands, are covalently attached to the dendrimers. In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the active agents in vivo. Both the composition of the linking moiety and its point of attachment to the active agent, are selected so that cleavage of the linking moiety releases either an active agent, or a suitable prodrug thereof. The composition of the linking moiety can also be selected in view of the desired release rate of the active agents.

In some embodiments, the attachment occurs via one or more of disulfide, ester, ether, thioester, carbamate, carbonate, hydrazine, or amide linkages. In preferred embodiments, the attachment occurs via an appropriate spacer that provides a disulfide bridge between the agent and the dendrimer. In this case, the dendrimer complexes are capable of rapid release of the agent in vivo by thiol exchange reactions, under the reduced conditions found in body.

Linking moieties generally include one or more organic functional groups. Examples of suitable organic functional groups include secondary amides (—CONH—), tertiary amides (—CONR—) , secondary carbamates (—OCONH—;—NHCOO—), tertiary carbamates (—OCONR—;—NRCOO—), ureas (—NHCONH—;—NRCONH—;—NHCONR—,—NRCONR—), carbinols (—CHOH—,—CROH—), disulfide groups, hydrazones, hydrazides, ethers (—O—), and esters (—COO—,—CH₂O₂C—, CHRO₂C—), wherein R is an alkyl group, an aryl group, or a heterocyclic group. In general, the identity of the one or more organic functional groups within the linking moiety can be chosen in view of the desired release rate of the active agents.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains.

The linking moieties may be designed to provide a releasable or non-releasable form of the dendrimer complex in vivo. For the releasable constructs, exemplary linking moieties include, but are not limited to, disulfide, ester, carbonate, carbamate, thiol, thioester, cathepsin sensitive, hydrazine, glucuronide bond, hydrazides, N-alkyl, ethyl, hydroxymethyl, and amide linkages. For the non-releasable constructs, exemplary linking moieties include, but are not limited to, ether, thioether and maleimidocaproyl linkages. In some embodiments, no linker is present in between the agent/ligand and the hydroxyl group of 2-deoxy-glucose.

In some embodiments, the dendrimer complexes described herein are used as hydrogels, linear polymers, hyperbranched polymers, glycopolymers (eg hyaluronic acid, chitosan, dextran etc), biopolymers, implants, or connected to other nano or micro-technologies through cross-linking reaction.

The dendrimer complex may comprise one or more biologically active agents, imaging agents, diagnostic agents, targeting ligands, E3 ligase, PROTAC, biologics (siRNA, mRNA, peptides, oligonucleotides, microRNA, genes, antibodies, radioligands, radiotherapeutics, PET agents) encapsulated, associated, and/or conjugated in the dendrimer complex at a concentration of about 0.01% to about 30%, preferably about 1% to about 20%, more preferably about 5% to about 20% by weight. The dendrimer can be conjugated to more than one agent and more than one type of agent.

Exemplary diagnostic materials include paramagnetic molecules, fluorescent compounds, magnetic molecules, and radionuclides. Suitable diagnostic agents include, but are not limited to, x-ray imaging agents and contrast media. Radionuclides also can be used as imaging agents.

Exemplary radioactive label include ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²p, ¹⁸⁶Re, ³⁵S, ⁷⁵Se, ¹⁷⁵Yb. Examples of other suitable contrast agents include gases or gas emitting compounds, which are radioopaque. In some embodiments, the imaging agent to be incorporated into the dendrimer nanoparticles is a fluorophore (e.g., fluorescein isothiocyanate (FITC), phycoerythrin (PE)), an enzyme (e.g., alkaline phosphatase, horseradish peroxidase), element particles (e.g., gold particles).

Further agents/groups/ligands that may be encapsulated, associated, and/or conjugated in the dendrimer complex described herein include any other sugars or non-sugar groups including drugs (for example: kinase inhibitors, RTK inhibitors, statins, anti-inflammatory, anti-oxidants, ant-viral, anti-VEGF, angiogenesis inhibitors, antiproliferative inhibitors, apoptosis inhibitors, autophagy inhibitors, trail-agonists, BET inhibitors, Bcr-Abl kinase inhibitors, anti-cancer, mTOR inhibitors, proteosome inhibitors, PARP inhibitors, JAK inhibitors, PPAR-gamma agonists/antagonists, anti-diabetic, galantamine, cabozantinib, AChE-inhibitor, CSDF-1R inhibitors, cannabinoids, ALK kinase inhibitor-1, anaplastic lymphoma kinase (ALK) PROTAC, Beta-2 Adrenergic Receptors: beta-2 adrenergic receptor (ADRB2) agonist, β2-adrenergic receptor blocker, adiponectin receptor (AdipoR) agonist, β3 adrenergic receptor antagonist, β-arrestin/β2-adaptin interaction inhibitor, β-adrenoceptor antagonist, a-adrenergic receptor agonist, muscarinic-3 (M3) agonist, CXCR2 antagonist, CXCR6 antagonist, phosphodiesterase-4 (PDE4) inhibitor, tumor necrosis factor-α (TNF-α) inhibitor, antagonist of H1-histamine receptor, TYK2 inhibitor, TIE-2 inhibitors, 5-HT transporter inhibitor, histamine H3 receptor full antagonist, histamine H1-receptor antagonist, histamine H2-receptor antagonist, histamine H1 and H2 receptor agonist, antihistamine agent, EGFR Inhibitors, PDGFR Tyrosine Kinase Inhibitor III, FAK dual inhibitor, STAT3 Inhibitors, BRD4 inhibitor, RAS inhibitor, glutathione peroxidase 4 (GPX4) inhibitor, c-Myc inhibitor, hCYP1B1 inhibitor, BACE-1 inhibitor, TGF-β receptor kinase inhibitor, ROS1 kinase inhibitor, VEGFR inhibitors, (Vascular Endothelial Growth Factor Receptor), endothelin receptor antagonist, cystic fibrosis transmembrane regulator, ATP-sensitive K+channel (KATP) inhibitor, TGF-beta/Smad inhibitors, ROCK inhibitors, interleukin-6 (IL-6) receptor antagonist, Toll-like receptor 7 and 8 (TLR7/TLR8) agonist, IRAK4 (Interleukin 1 receptor associated kinase 4) inhibitor, human formyl peptide receptor like-1 (FPRL-1/FPR2) agonist, nuclear factor-kappa B (NF-κB) activators/inhibitors, tumor necrosis factor-α (TNF-α) inhibitor, matrix metalloproteinases (MMP) inhibitor, 5-lipoxygenase (5-LO) inhibitor, antagonist of prostaglandin E2 (PGE2) receptor (EP 1), prostanoid receptor ligand, androgen receptor inhibitor, ROR agonist-1, retinoic acid receptor-related orphan receptor γt (RORγt), IL-17A inhibitor, IL-17 receptor inhibitor, GABA (A) receptors competitive antagonist, GABA aminotransferase activator, Thromboxane receptors antagonists or synthase inhibitors, angiotensin II AT2 receptor agonist, matrix metalloproteinase-2 (MMP-2) selective inhibitor, CGRP receptor activator, antagonist of CGRP receptor, adrenomedullin receptor antagonist, calcium and sodium channel blockers, Beta blockers, Tumor Necrosis Factor-alpha (TNF-alpha) inhibitors (such as Infliximab, Adalimumab, and Etanercept), Interleukin-1: IL-1 inhibitors like Anakinra and Canakinumab, Interleukin-6 (IL-6) Inhibitors like Tocilizumab and Sarilumab, B-cell lymphoma inhibitors, Immunosuppressants, T cell modulator like Efalizumab, IL-23 and IL-12 inhibitors, C5a Receptor agonist, RANKL (Receptor Activator of Nuclear Factor Kappa-B Ligand) inhibitors like Denosumab, Thyroid Hormone Receptor Antagonists, PPARα agonist, Angiotensin Inhibitors, Adenosine A1/A3 Receptor Antagonist, u-opioid receptor antagonist, antagonist for the κ-opioid receptor, μ opioid receptor partial agonist, kappa opioid receptor (KOR) agonist, competitive antagonist of the NOP receptor, pan-opioid antagonist, δ-opioid receptor (DOR) antagonist, voltage-gated sodium channel (NaV) 1.7 inhibitor, inhibitor of noradrenaline transporter (NET), voltage-gated potassium channel blockers, CGRP receptor activator, antagonist of serotonin receptors, agonist for melatonin receptors, EP3 receptor agonist, EP1- and EP3-receptor agonist, P2X3 receptor (P2X3R) antagonist, inhibitor of P2X3 receptor, purinergic (P2X1) receptor antagonist, selective and non-nucleotide antagonist of P2X3 and P2X2/3 receptors, protease-activated receptor (PAR-2) agonist, orexin receptor antagonists, Tropomyosin-related kinases, COX-3 (Cyclooxygenase-3) inhibitors, diazepam binding inhibitor (DBI) receptor, agonist of benzodiazepine receptor, corticotropin-releasing hormone receptor 1 (CRHR1) antagonist, Inhibitor of β-Secretase and voltage-gated sodium channel, antagonist of metabotropic glutamate receptor type 1 (mGluR1), psychedelics, ligands, dyes, radioligands, siRNA, targeting ligands, bioactive groups, folic acid, polymers, chelating agents (e.g. DOTA, NOTA), or inorganic molecules etc), Oligonucleotides, inhibitor of HBV replication, HBV DNA replication inhibitor, HBV capsid assembly modulator, surface antigen (HBsAg) inhibitor, HBV transcriptional suppression, HBV capsid inhibitor, protein assembly modulator, inhibitor of HBV DNA polymerase, HIV infection inhibitor, HIV infection inhibitor, anti-HBV/HCV/HSV-1/HIV, anti-inflammatory, selective FXR agonist, antiretroviral drug-resistant HIV strains, anti-bacterial agent, Sirtuin 5 deacylase inhibitor, Sirtuin 5 deacylase inhibitor, Sirtuin-1 (SIRT1) activator, DNA methyltransferase 3A (DNMT3A) inhibitor, methyltransferase (DNMT1) selective inhibitor, MGMT (06-methylguanine DNA methyltransferase) inhibitor, thymidylate synthase inhibitor, G9a/DNA methyltransferases (DNMTs) inhibitor, non-covalent inhibitor for DNA methyltransferase (Dnmt1), inhibitor of histone deacetylase and DNA methyltransferase, inhibitor of DNA (cytosine-5)-methyltransferase (Mtase), acetyl-CoA carboxylase (ACC) inhibitor, ACC2 inhibitor, inhibitor of NLRP3 inflammasome, selective connexin 43 (Cx43) hemichannel blocker, connexin 43 (gap junction blocker), connexin 26 (Cx26) inhibitor, antibacterial against Gram-positive, Gram-negative and anaerobic organisms, inhibitor of DNA gyrase and topoisomerase IV activity, activator of peroxisome proliferator-activated receptor-y coactivator-1a (PGC-1α), Liver kinase B1, AMP-activated protein kinase (AMPK) activator, Cystic fibrosis transmembrane conductance regulator (CFTR), glucagon receptor antagonist, glucagon-like peptide-1 receptor (GLP-1-R) antagonist, dipeptidyl peptidase IV (DPP-IV) inhibitor, cytotoxic tubulin modifier, inhibitor of catecholamine release, Estrogen receptor antagonist 3, TXNIP inhibitor, GLP-1 receptor agonist 10, neurokinin 2 (NK2) receptor agonist, glucose-dependent insulinotropic polypeptide (GIP) and glucagon-like peptide-1 (GLP-1) receptor agonist, NADH: ubiquinone oxidoreductase inhibitor, G-protein-coupled receptor kinase 5 (GRK5) inhibitor, amylin receptor antagonist, IRE-1α inhibitor, IRE-1a inhibitor, flukicidal agent against liver flukes, Glucocorticoid receptor agonist, melanocortin MC4 receptor antagonist, anti-rabies virus (RABV), melanocortin MC4 receptor antagonist, stimulator of interferon genes (STING) agonists, alpha 1-adrenoceptor blocker, alpha 1-adrenoceptor blocker, inhibitor of muscarinic acetylcholine receptor (mAChR), μ-opioid receptor antagonist, potent tubulin polymerization inhibitor, antioxidant, anti-psychotic, anti-convulsant, Parkinson drugs, Alzheimer drugs, Narcotic analgesics (pain relievers), Nonnarcotic analgesics (such as acetaminophen and NSAIDs), and Antiemetics.

Embodiments provide methods for delivering agents across the blood-brain barrier in a subject in need thereof by the administration of a dendrimer complex as described herein.

Embodiments provide methods for treating or ameliorating neurological conditions, diseases, or disorders by the administration of a dendrimer complex as described herein. As used herein “treating” or “treatment” means (a) inhibiting the disease, i.e. slowing or halting the development of clinical symptoms; and/or (b) alleviating the disease, i.e. causing regression of clinical symptoms and/or or (c) any treatment of the disease, including alleviation or elimination of the disease and/or its attendant symptoms.

The compositions are suitable for treating one or more diseases, conditions, and injuries in the brain and the nervous system, particularly those associated with pathological activation of microglia and astrocytes.

In some embodiments, the compositions and methods are administered to a subject in need thereof in an effective amount to reduce, or prevent one or more molecular or clinical symptoms of a neurological disease, or one or more mechanisms that cause neurodegeneration.

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

In some embodiments, the subject has a nervous system disorder or is in need of neuroprotection. Exemplary conditions and/or subjects include, but are not limited to, subjects having had, subjects with, or subjects likely to develop or suffer from a stroke, a traumatic brain injury (TBI), chronic traumatic encephalopathy (CTE), a spinal cord injury, Post-Traumatic Stress syndrome, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease. Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wemicke-Korsakoff's syndrome, post-encephalitic dementia, cancer and chemotherapy-associated cognitive impairment and dementia, and depression-induced dementia and pseudodementia.

The dendrimer complexes can be administered in combination with one or more additional therapeutically active agents, which are known to be capable of treating conditions or diseases discussed above. In some embodiments, the dendrimer complex is administered with or without radiation therapy.

In some embodiments, the methods described herein include an initial step of determining if the subject has a neurological condition as described herein and if the subject is determined to have the neurological condition, then administering to the subject a therapeutically effective dose of a composition as disclosed herein.

The compositions described herein may be administered in vivo by any suitable route (e.g. parenterally or enterally) including but not limited to: inoculation or injection (e.g. intravenous, intraperitoneal, intramuscular, subcutaneous, intra-aural, intraarticular, intramammary, and the like), topical application, and by absorption through epithelial or mucocutaneous linings (e.g., nasal, oral, vaginal, rectal, gastrointestinal mucosa, and the like). Other suitable means include but are not limited to: inhalation (e.g. as a mist or spray), orally (e.g. as a pill, capsule, liquid, etc.), intravaginally, intranasally, rectally, by ingestion of a food or probiotic product containing the compound, as eye drops, etc. In preferred embodiments, the mode of administration is oral or by injection.

A patient or subject to be treated by any of the compositions or methods of the present disclosure can mean either a human or a non-human animal including, but not limited to mammals, dogs, horses, cats, rabbits, gerbils, hamsters, rodents, birds, aquatic mammals, cattle, pigs, camelids, and other zoological animals. In some embodiments, the subject to be treated is a child or an infant.

In some embodiments, the formulation or active agent is administered to the subject in a therapeutically effective amount. By a “therapeutically effective amount” or an “effective amount” is meant a sufficient amount to treat the disease or disorder at a reasonable benefit/risk ratio applicable to any medical treatment. It will be understood that the total daily usage of the compositions of the present disclosure will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. It is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 1 to 1,500 mg per adult per day. In particular, the compositions contain at least or up to 1.0, 2.5, 5.0, 10.0, 15.0, 25.0, 50.0, 100, 250, 500, 750, 1000, 1250, or 1500 mg of the active ingredient for the symptomatic adjustment of the dosage to the subject to be treated. A medicament typically contains from about 0.01 mg to about 1500 mg of the active ingredient, in particular from 1 mg to about 250 mg of the active ingredient. An effective amount of the drug is ordinarily supplied at a dosage level at least or up to 1 mg/kg to 100 mg/kg of body weight per day, e.g. about 5 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, or 100 mg/kg of body weight per day. Such doses may be administered in a single dose or it may be divided into multiple doses.

Further embodiments provide a method of imaging neurons in vitro or in vivo, comprising contacting the neurons with a complex comprising a dendrimer as described herein, wherein an imaging agent is conjugated to an outer surface of the dendrimer; and detecting the imaging agent.

Embodiments of the disclosure also provide pharmaceutical compositions comprising the dendrimer complexes described herein and a pharmaceutically acceptable carrier/excipient. The pharmaceutical compositions can be formulated according to known methods for preparing pharmaceutically useful compositions. The active ingredients may be mixed with excipients which are pharmaceutically acceptable and compatible with the active ingredients. Pharmaceutically” or “pharmaceutically acceptable” refer to molecular entities and compositions that do not produce an adverse, allergic or other untoward reaction when administered to a mammal, e.g. a human, as appropriate. As used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W Easton Pa., Mack Publishing Company, 19th ed.) describes formulations which can be used in connection with the subject invention. The final amount of the compounds in the formulations may vary. However, in general, the amount in the formulations will be from about 0.01-99%, weight/volume.

The dendrimer complexes described herein include the pharmaceutically acceptable salts thereof. “Salts” or “pharmaceutically acceptable salts” refers to the relatively non-toxic, inorganic and organic acid addition salts, and base addition salts, of compounds of the present disclosure. These salts can be prepared in situ during the final isolation and purification of the compounds. In particular, acid addition salts can be prepared by separately reacting the purified compound in its free base form with a suitable organic or inorganic acid and isolating the salt thus formed.

Compositions as described herein may be prepared either as liquid solutions or suspensions, or as solid forms such as tablets, pills, granules, capsules, powders, ampoules, and the like. The liquid may be an aqueous liquid. Solid forms suitable for solution in, or suspension in, liquids prior to administration may also be prepared. Example dosage forms include a tablet, dragee, liquid, drop, capsule, caplet, gelcap, etc.

Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question. The pharmaceutical composition can be adapted for various forms of administration.

Administration can be continuous or at distinct intervals as can be determined by a person skilled in the art.

The compositions of the present disclosure may also contain other components such as, but not limited to, additives, adjuvants, buffers, tonicity agents, bioadhesive polymers, and preservatives. In any of the compositions of this disclosure, the mixtures are preferably formulated at about pH 5 to about pH 8. This pH range may be achieved by the addition of buffers to the composition. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example 1

The availability of non-invasive drug delivery systems capable of efficiently transporting bioactive molecules across the blood-brain barrier to specific cells at the injury site in the brain is currently limited. Delivering drugs to neurons presents an even more formidable challenge due to their lower numbers and less phagocytic nature compared to other brain cells. Additionally, the diverse types of neurons, each performing specific functions, necessitate precise targeting of those implicated in the disease. Moreover, the complex synthetic design of drug delivery systems often hinders their clinical translation. The production of nanomaterials at an industrial scale with high reproducibility and purity is particularly challenging. In this study, we have developed a third-generation 2-deoxy-glucose functionalized mixed layer dendrimer (2DG-D) utilizing biocompatible and cost-effective materials via a highly facile convergent approach, employing copper-catalyzed click chemistry. We further evaluated the systemic neuronal targeting and biodistribution of 2DG-D, and brain delivery of a neuroprotective agent pioglitazone (Pio) in a pediatric traumatic brain injury (TBI) model. The 2DG-D exhibits favorable characteristics including high water solubility, biocompatibility, biological stability, nanoscale size, and a substantial number of end groups suitable for drug conjugation. Upon systemic administration in a pediatric mouse model of traumatic brain injury (TBI), the 2DG-D localizes in neurons at the injured brain site, clears rapidly from off-target locations, effectively delivers Pio, ameliorates neuroinflammation, and improves behavioral outcomes. The in vivo results coupled with a convenient synthetic approach for the construction of 2DG-D makes it a useful nanoplatform for addressing brain diseases.

### Methods

### Materials and Reagents

PHEMS buffer, PBS, Poly-L-lysine, 4′,6-diamidino-2-phenylindole (DAPI), 3-(4,5-dimethylthiazol-2-yl)-2,5 diphenyltetrazolium bromide (MTT), and Triton® X-100 were procured from Aaron Chemicals. The cell painting kit and Pheno Vue® neuronal differentiation staining kit were purchased from Revvity. CATH.a cell line was obtained from ATCC, United States. DMEM, RPMI-1640 was purchased from Cytiva, FBS was obtained from Gibco Scientific and DAPI-cell mounting medium was obtained from Vectorlabs. Rat red blood cells (RBCs) were purchased from Innovative Research. All these above-mentioned reagents were used as such.

### In Vitro Studies-Experimental Section:

Primary culture of the cortical neurons: The cortical neurons were isolated as per the described procedure. Briefly, the brain tissue from the Sprague Dawley rats was collected at post-natal day 0. The dorsal and frontal regions of the cortex were then dissected and incubated in the buffer for digestion. The buffer contained 10 unit/mL papain, 100 unit/mL DNase I, and 5 mg/mL cystine, in Hibernate A nutrient medium. After 30 min of the digestion, the neurons were further seeded on poly-D-lysine (50 μg/ml)-coated plates (1.5×10⁵) in a 24 well plate. For culturing of the cortical cells, the medium was Neurobasal A with B27 supplement with 1% anti-anti and glutamate. The cells were then directly used for further experiments.

Cellular compatibility/MTT assay using primary and secondary neuronal cultures: For evaluating the applicability of the developed 2DG-D dendrimers for in vivo studies, these were first evaluated under in vitro settings with both cortical and CATH.a neuronal cells. In this assay, briefly 1×10⁵ cortical neuron cells and 1×10⁴ CATH.a neurons were plated in a 24 well and 96 well plate, respectively, in presence of the respective growth media with 10% FBS supplement. Following this, the cells were grown overnight in a CO₂ incubator with 5% CO₂ at 37° C. The media was removed further, and the corresponding cells were treated with different dendrimers and control samples along with the fresh media for 24 and 48 h. Afterwards, the media was removed, and the cells were carefully washed thrice by 1X PBS (10 mM, pH 7.4). Following this, 10 μl MTT (5 mg/mL) was added to each of the wells and incubated for almost 3 h at 37° C. in a CO₂ incubator. All the media was aspired carefully, and to each well 150 μL DMSO was added and incubated for 15 min for proper dissolution of the formazan aggregates. Finally, the cell viability was calculated by recording the absorbance at 570 nm using multi-mode microplate reader (Thermo Scientific Multiskan® SkyHigh® Microplate Spectrophotometer). All the samples were done in triplicates with proper controls. The cell viability (%) was calculated by equation (1).

$$Cell viability ( \% ) = \frac{A b s o r bance of Test sample}{A b s o r bance of Control} \times 100 \qquad ( 1 )$$

Cell viability studies using macrophages: 2.5×104 RAW Blue macrophages were allowed to adhere to a 96 well plate overnight. The following day, cell media was changed and the 2DG-D dendrimers at the concentrations tested were added. The cells were allowed to incubate at 37° C. for 48 h. Following incubation, the luminescence of the viable cells was measured using the CellTiter-Glo® Luminescent Cell Viability assay according to manufacturer's instructions. Cell viability (%) was calculated using the luminescence values obtained from the controls used in the experiment. The experiment was performed in triplicate.

Cellular internalization/uptake and blocking studies of 2DG-D-Cy5: In order to evaluate the uptake of 2DG-D-Cy5 dendrimers by the cells, cellular internalization studies were carried out with both primary isolated cortical neurons and CATH.a neurons. Both these cell lines were employed to increase the mimic for in vivo studies for TBI mouse model. First, qualitative uptake studies were carried out only with the primary isolated cortical neurons to optimize the dendrimer concentration for the uptake studies. For this, the isolated cortical neurons were seeded in a 24 well plate having poly-L lysine coated cover glasses with a seeding density of 1×10⁵ cells/well. The cells were grown for 24 h at 37° C. with 5% CO₂ and 95% moisture, in a CO₂ incubator. The media was removed after 24 h and the cells were washed with 1×PBS. Different concentrations (100, 50, 25, 12.5, 6.3 and 3.1 μg/mL) of 2DG-D-Cy5 was added to the respective wells with the fresh media and the cells were incubated for 12 h in the CO₂ incubator. The cells were washed again with chilled 1× PBS followed by treatment with 10 μL of (50 μM) DAPI for 20 min. For properly fixing the cells, the media was again aspirated, and wells were washed multiple times with chilled 1× PBS. Afterwards 1 mL of PHEMS buffer containing 4% paraformaldehyde was added and incubation was done for 20 mins. The cells were finally washed with cold PBS and mounted on the clean glass slide using mounting media. Imaging was carried out on Leica, SP-5 confocal laser scanning microscope at 20× magnification with water immersion. A similar study was performed with cortical neurons and CATH.a cells in presence of cell trafficking inhibitors viz., chlorpromazine (CPZ, inhibitor for clathrin-mediated endocytosis), methyl β-cyclodextrin (MBCD, inhibitor for caveolae-mediated endocytosis), cytochalasin B (GLUT-inhibitor) and phloretin (GLUT inhibitor). For this, the overnight grown cells on the poly-L lysine coated cover glasses in 24 well plate were treated with CPZ (10 μg/mL, 30 μM), MBCD (2.5 mg/mL, ˜2 mM), cytochalasin B (10 μg/mL, 20 μM) and phloretin (300 μg/mL, ˜1 mM) for 1 h at 37° C. in a CO₂ incubator. The inhibitors were aspirated, and the cells were carefully washed with 1×PBS thrice and further incubated with 2DG-D-Cy5 for 4 h at 37° C. and then washed again with chilled 1× PBS. Further, the cells were fixed by 4% paraformaldehyde (equal volumes of 8% paraformaldehyde and 2× PHEMS buffer at room temperature for ˜20 minutes. The fixative was aspirated, and the cells were washed multiple times with cold 1× PBS. The fixed cells were permeabilized by treating with 0.2% of triton-X-100 for ˜10 min and washing with cold PBS again. For CATH.a neuronal culture, the staining was carried out with 10 μL of (50 μM) DAPI for 20 min, followed by washing and staining with 20 μL of PhenoVue® Fluor 568-Phalloidin (˜0.4 nM) for 1 h, followed by washing with cold 1× PBS and imaging was carried out on Leica, SP-5 confocal microscope. In contrast to CATH.a neurons, for primary cortical neurons immunofluorescence staining procedure was carried out to specifically probe and differentiate the neurons from the glial cells present in the primary culture. For this, the Triton®-X-100 permeabilized cells were blocked with 5% bovine serum albumin (1× PBST) for 1 h at RT to limit the false positive staining. After blocking was done, the solution was aspirated, and cells were washed multiple times with 1×PBST. The blocked cells were incubated with primary anti-Nestin antibody (1:100) and kept at 4° C. overnight. After primary antibody treatment, the cells were thoroughly washed using PBST on a dancing shaker for 15 min (3 washes). For probing Nestin the cells were incubated with Pheno Vue® Fluor 488-Rat anti-mouse IgG1 secondary antibody for 2 h at 37° C. Following this, the cells were again washed using 1× PBST thrice (3×5 min) and stained with DAPI as mentioned above. The cover glass containing the stained cells were finally mounted on the cleaned glass slides using mounting medium and imaging was carried out on Leica, SP-5 microscope as mentioned above. All the measurements were done in replicates with proper controls.

### Ex Vivo Dendrimer Quantification.

The frozen organs (heart, lungs, liver, kidneys, spleen, and brain) were gradually thawed on ice, and weighed. The tissue samples were dissected to measure known amounts of tissues from each organ. The brain was dissected to separate the injured and non-injured regions. The tissue samples underwent homogenization with stainless steel beads in methanol at a 1 ml: 100 mg tissue ratio using a tissue homogenizer. The homogenized samples were centrifuged at 4° C. and the clear supernatant was transferred to protein LoBind Eppendorf tubes and stored at −80° C. For fluorescence quantification, the thawed supernatants were centrifuged again, fluorescence intensity was measured using Fluoromax® spectrofluorophotometer. Fluorescence intensities for Cy5 (2ex=645 nm, hem =662 nm) were determined and were adjusted for background fluorescence from control tissue. The fluorescence intensity values were converted to 2DG-D-Cy5 concentrations using calibration curves of 2DG-D-Cy5 at different slit widths. Serum, diluted 10-fold in Dulbecco's PBS, was also measured after filtration.

### Hemolysis/Hemocompatibility Assay.

With the rationale of using these molecules for in vivo studies, the hemolysis assay was carried out to see the effect of 2DG and 2DG-D-Pio on the rat red blood cells (RBCs). In brief, ˜5 mL of rat RBCs were diluted with 15 mL of 1×PBS (pH 7.4). Further, 250 μL of the RBC solution was added to 250 μL of 2DG or 2DG-D-Pio dendrimer at different concentrations (5, 2.5, 1.25, 0.63 and 0.31 mg/mL) in a micro centrifuge tube. All these samples were then incubated for almost 3 h at 37° C. in an incubator shaker at a rotation of ˜80 rpm. Further, the resulting solution was carefully centrifuged at 5000 rpm for 10 min at room temperature and ˜200 μL of supernatant from each of the dendrimer treated RBC samples was taken into a 96 well plate for evaluating the absorbance values recorded at 540 nm which corresponds to the hemoglobin release from the RBCs, using Thermo Scientific Multiskan® SkyHigh® Microplate reader. The RBCs that were treated with the 1× PBS (10 mM, pH 7.4) were kept as a negative control, whereas 1% Triton®-x-100 treated RBCs were taken as positive control for hemolysis. From the recommendation by ASTM E2524-08 standard, any materials showing less than 5% hemolysis can be considered as hemocompatible. The percentage hemolysis was calculated by following equation (2).

$$Hemolysis ( \% ) = \frac{\begin{matrix}( Absorbance of test sample - \\ Absorbance of negative control )\end{matrix}}{\begin{matrix}( Absorbance of positive control - \\ Absorbance of negative control )\end{matrix}} \times 100 \qquad ( 2 )$$

In vivo Studies-Experimental Section:

### Biosafety Studies.

Animal studies: To investigate systemic toxicity, 3 weeks-old C57BL/6 mice (3 males and 3 females per group) were purchased from Jackson Laboratory. After 1 week of acclimation, mice were randomly separated into 3 groups: 1) Saline group: mice were i.p. injected with saline as blank control. 2) 2DG-D-Pio group: mice were i.p. injected with 2DG-D-Pio at the dose of 5 mg/kg Pio equivalent. 3) 2-DG-D group: mice were i.p. injected with equivalent 2-DG-D dendrimer solution as control dendrimer platform. All three groups were administered as a single intraperitoneal injection. Mice were weighed daily for 3 days after drug injection. On day 3, the liver and kidney were harvested for histological analysis. Blood was collected and then centrifugated at 3000 rpm for 15 min. The serum was collected for biochemical analysis following the kit instructions, including Alanine aminotransferase (ALT, BioAssay System, EALT-100) and Aspartate aminotransferase (AST, BioAssay System, EASTR-100), creatinine (CRE, BioAssay System, DICT-100), and urea nitrogen (BUN, BioAssay System, DIUR-100), following the assay instructions.

Histology Examination: Mouse liver and kidney tissues were fixed in 4% v/v phosphate-buffered formaldehyde, embedded in paraffin, sectioned, and stained with hematoxylin and eosin (H&E).

### Efficacy Studies

### Animals

Male and female C57BL/6 mice (2-3 month of age; Jackson Laboratory, Bar Harbor, ME) were in-house bred. All of the pups were delivered naturally and remained with their mother after birth until weaning. All animals were housed under ambient conditions (20-22° C., 40-60% relative humidity, and a 12-h light/dark cycle) with free access to food and water. Multiple precautions, including adequate habituation, gentle handling, minimization of procedure duration, and the use of humane endpoints according to “Recognition and Alleviation of Distress in Laboratory Animals”, were taken throughout the study to minimize pain and stress associated with experimentation. All experiments followed the Guide for the Care and Use of Laboratory Animals, eighth edition, published by the National Research Council (National Academies Press, 2011). Experimental procedures were approved by the Institutional Animal Care and Use Committee (IACUC) of the University of Michigan.

### Impact Acceleration Model of TBI

On postnatal day 20-21 (P20-21), male (M) and female (F) animals (n=73, 38M/35F) from the same litter were randomized into Sham (n=17, 9M/8F) and TBI (n=52, 29M/27F) groups using a random number generator. Randomization was stratified by sex. Anesthesia was induced with 4% isoflurane and tail and/or paw pinches were used to ensure the animal was fully sedated. The TBI animals underwent injury procedure as previously described. In brief, anesthesia was induced with 4% isoflurane and tail and/or paw pinches were used to ensure the animal was fully sedated. After fully anesthetized, the animal was placed chest-down on a platform with a trapdoor that supported the body weight of a mouse (˜7-10 g body weight) with little to no resistance or restraint upon impact. The animal's head was directly in the path of a falling weight. A weight (30 g) was held at 1.0 meter above the platform and secured by a pin. The lab personnel pulled the pin, allowing the weight to fall vertically through a guide tube to strike the animal on the head in the midline between bregma and lambda (at approximately bregma-2.5 mm). The animal rapidly underwent a 180° rotation, falling through the trapdoor and landing in a supine position on a cushion. The animal was removed immediately from the apparatus and placed in a clean warm cage. Sham animals were anesthetized with 4% isoflurane without TBI impact. All animals were closely monitored postoperatively with weight and health surveillance recordings, as per IACUC guidelines.

2DG-D Co-Localization with Neurons

Male and female TBI mice (n=2 per sex) received intraperitoneal administration of fluorescent 2DG-D (50 mg/kg, 100 μL) at 6-h post-injury, and euthanized at 24-h post-injection. Brains were removed, postfixed in 10% formalin for 48 h, and then cryoprotected in 30% sucrose (in PBS). Coronal sections (20 μm, 1:6 series) were prepared on a cryostat (Leica Microsystems, IL, USA). Brain sections were incubated overnight at 4° C. with rabbit anti-NeuN (a neuronal marker; 1:250, Abcam, MA. U.S.A.) or rabbit anti-IBA1 (a microglial marker; 1:250, FUJIFILM Wako Chemicals U.S.A. Corporation, VA. U.S.A). Sections were subsequently washed and incubated with fluorescent secondary antibodies (1:250; Life Technologies, MA, U.S.A.) for 2 h at room temperature. The slides were dried, and cover slipped with fluorescent mounting medium with DAPI (Sigma-Aldrich, MO, USA). Images were acquired using Nikon Eclipse® TS2R fluorescent microscope (Nikon, NY, USA).

### Bio Distribution Study of 2DG-D-Cy5

The mice from the same litter (n=24, 12M/12F) were randomly divided into sham (n=6, 3M/3F) and TBI (n=18, 9M/9F) groups. The animals received intraperitoneal administration of 2DG-D-Cy5 (50 mg/kg, 100 μL) at 6-h post-injury. Mice in the TBI group were euthanized at 1, 3 and 24-h post-injection (n=6, 3M/3F, per time point). Mice in the sham group were euthanized at 24-h (n=6, 3M/3F) post-injection. Animals were transcardially perfused with PBS. The brain (injured regions and non-injured regions), heart, lungs, liver, spleen, kidneys, plasma, and urine were harvested.

### In Vivo Pioglitazone/Dendrimer-Pioglitazone Administration

Mice in the TBI group were randomized into TBI+saline (n=11, 6M/5F), TBI+pioglitazone (TBI+Pio) (n=11, 6M/5F), and TBI+Dendrimer-pioglitazone (TBI+2DG-D-Pio) (n=12, 6M/6F) groups. Animals received intraperitoneal administration of free Pio (5 mg/kg, 100 μL), 2DG-D-Pio (containing 5 mg/kg pioglitazone, 100 μL) or PBS (100 μL) at 6-h post-injury. The mice from the sham group (n=11, 6M/5F) did not receive any intervention.

### Body Weight

Body weight was measured before injury (baseline) and at 1-day (d) post-treatment. The changes in the body weight were calculated as (Body weight)_{change}=(body weight) 1d-(body weight)_{baseline}.

### Behavioral Tests

All of the behavioral testing was performed between 7AM to 6 PM. Mice were habituated in the test room for at least 30 minutes before the behavioral tests. The lab personnel were blinded to experimental groups.

Grip strength: Muscular strength was evaluated with a grip strength test using a grip strength meter (BIOSEB, FL, USA) before injury and at 1-d post-treatment. In brief, the grip strength meter was positioned horizontally, and the animals were held by the tail and lowered towards the apparatus. The animals were allowed to grab the metal grid and were then pulled backwards in the horizontal plane. The force applied to the grid was recorded as the peak tension. Each animal underwent a grip strength test in three consecutive trials. The results were recorded and averaged for each animal. The change in the grip strength before and after injury was calculated as: (grip strength)_{change}=(grip strength)₂₄ₕ-(grip strength)_{baseline}

Rotarod: Sensorimotor coordination, endurance, and fatigue resistance was evaluated with a touchscreen five station accelerating Panlab RotaRod for mouse (BIOSEB, FL, USA) before injury and at 1-d post-treatment based on a published protocol. Each animal was situated on a stationary rod for 10 s, and the rod was then set in motion with an accelerating speed of 3-30 rpm. Each animal underwent three consecutive trials (5 min each). The latency to the first fall in each trial was recorded and averaged for each animal. The change in the latency to the first fall before and after injury was calculated as:

(Latency)_{change}=(latency)₂₄ₕ−(latency)_{baseline}

Tail suspension test: The tail suspension test was performed at 1-d post treatment as previously described to evaluate depression-like behaviors. In brief, mice were suspended by taping their tails (three quarters of the distance from the base of the tail) to a vertical bar on a tail suspension stand. The animal tail was aligned with the bottom of the bar. The animals' activities were monitored continuously for 6 min. The time spent immobile over the 6 min period were quantified and compared among groups.

Light/dark box test: The light/dark box was purchased from Stoelting Co. (Wood Dale, IL, USA), and the test was modified from published protocols to evaluate anxiety-like behaviors. The test was performed at 1-d post treatment. In brief, mice were placed in the middle of the brightly illuminated chamber and were allowed to move freely between the light and dark chambers for 10 min. Video recording was used to record animal behaviors. The time spent in the light chamber and the number of transitions between the light and the dark chambers was recorded and analyzed.

Novel object recognition: The novel object recognition test was modified from published protocols and performed at 1-d post treatment. In brief, the test was composed of two trials. The mice explored two identical objects for 5 min during the “training trial” and then were placed back in their cages. After an inter-trial break of 4-h, one of the previously exposed “old” objects was replaced with a new “novel” object, and the animals were allowed to explore these two objects for 5 min during the “probe trial”. The discrimination index for the probed trial was used to analyze the cognitive outcomes. Discrimination index=time spent exploring the novel object/(time spent exploring the old object+time spent exploring the novel object)×100%.

### Isolation of Primary Neurons

Primary neuron isolation was modified from a published protocol. In brief, brains were harvested and rinsed in HBSS solution on ice. Meninges were removed and the area of injury (approximately between bregma+2 mm and bregma-1 mm) and the area of non-injury (approximately between bregma-1 mm and bregma-3 mm) in the TBI mice, and the matching area in the sham mice were micro-dissected as previously described. Brain tissues were transferred to HABG solutions [60 mL HA, 1.2 mL B27, 0.176 mL Gln (0.5 mM final)], and minced (˜0.5 mm) on ice. Brain tissues were incubated in HABG solution for 8 min at 30° C. in a Boekel shaking incubator with a shaking speed of 90 rpm (Cole-Parmer, Vernon Hills, IL, USA). Tissues were transferred to papain solutions [12 mg papain solids per 6 mL HA-Ca, 0.015 mL Gln (0.5 mM final)], and incubated for 30 min at 30° C. in a shaking incubator with a shaking speed of 90 rpm. Tissues were washed in HABG solution for 5 min at room temperature, triturated with sterile pipette for 45 s, and sit at room temperature for 1 min. The supernatants were collected, and the trituration was repeated two times. The supernatants were combined and centrifuged in OptiPrep™ Density Gradient Medium at 800 xg for 15 min at 22° C., and the fractions of neurons were collected as previously described. Cells were washed in HABG solutions and centrifuged at 200×g for 2 min at 22° C. Supernatants were removed, and cells were washed in HBSS solution and centrifuged at 200×g for 2 min at 22° C. Cell pellets were harvested for RNA isolation.

RNA Isolation and Quantitative Real-Time Polymerase Chain Reaction (qPCR)

The mRNA expression of TNF-α (tumor necrosis factor alpha), IL-1β (interleukin-1 beta), IL-4, IL-6, IL-10, IL-13, TGF-β1 (transforming growth factor beta 1), iNOS (inducible nitric oxide synthase), NLRP3 (NLR Family Pyrin Domain Containing 3), and TLR4 (Toll-like receptor 4) were measured. The neurons isolated from the injured brain tissues (or the matching area of sham) were micro-dissected for RNA isolation as previously described. Total RNA was extracted using TRIZOL (Sigma-Aldrich, MO, USA), according to manufacturer's instructions. RNA samples were quantified using the Nanodrop® ND-2000 Spectrophotometer (Thermo Fisher Scientific, MA, USA). Single-stranded complementary DNA (cDNA) was reverse transcribed from RNA using the High-Capacity cDNA Reverse Transcription Kit with RNase inhibitor (Thermo Fisher Scientific, MA, USA). qPCR was performed with iTaq (tm) Universal SYBR® Green Supermix (Bio-Rad, CA, USA) with CFX connect real-time PCR detection system (Bio-Rad, CA, USA). Amplification conditions included 30 sec at 95° C., 40 cycles at 95° C. for 5 sec, and 60° C. for 30 sec. Primers were custom designed and ordered from Integrated DNA Technology (Coralville, IA, USA). The comparative threshold cycle (Ct) method was used to assess differential gene expressions. The sham group was the reference group, and glyceraldehyde 3-phosphate dehydrogenase (Gapdh) was the housekeeping gene. Gene expression levels for each sample were normalized to the expression level of Gapdh within a given sample (ΔCt); the differences between sham and TBI groups were used to determine the ΔΔCt. The 2-ΔΔCt gave the relative fold changes in gene expression.

### Statistical Analysis

Data were analyzed using GraphPad Prism® 6 (Version 6.04; CA, USA). All data were presented as mean ±SEM. D′agostino and Pearson omnibus normality test was used for normality measurement. Two-way ANOVA and Bonferroni post hoc tests were used for multiple group comparisons. Statistical significance was set at p<0.05 for all analyses.

### Results and Discussion

We rationally developed the 2DG-D guided by the following principles. (1) We utilized biocompatible building blocks and Generally Recognized As Safe (GRAS) reagents, with approximately 17% 2DG, 57% polyethylene glycol (PEG), 13% triazole, 9% core, and 4% gallic acid composition. (2) We incorporated PEG and 2DG-based building blocks, which are inexpensive and non-toxic. (3) We employed an efficient convergent synthetic approach to reduce synthetic complexity. (4) We created the 2DG-D with neutral surface charge and small size for enabling extravasation through impaired blood vessels and easy movement in the brain tissue parenchyma. The 2DG-D is a generation-3 mixed-layer glycodendrimer synthesized in an expedited manner using a combination of hypermonomer strategy, convergent synthesis, and highly efficient and orthogonal copper (I) catalyzed click chemistry (CuAAC) approach. The innermost layer, the core, comprises alkyne-terminating generation-1 PAMAM dendrimer, followed by a second layer composed of gallic acid building blocks, and the outermost layer consists of 2-DG. In contrast to the conventional method of dendrimer synthesis, where identical building blocks are employed sequentially throughout the generations, our approach involves utilizing a diverse combination of building blocks. This synthetic method offers us the flexibility to efficiently create dendrimers while allowing us to achieve entirely distinct structures with ease simply by altering the arrangement of building blocks within a given generation. The 2DG-D is highly soluble in water and there are 72 hydroxyl groups on the surface which can be easily manipulated to attach bioactive molecules of interest.

### Synthesis and characterization of 2DG-D

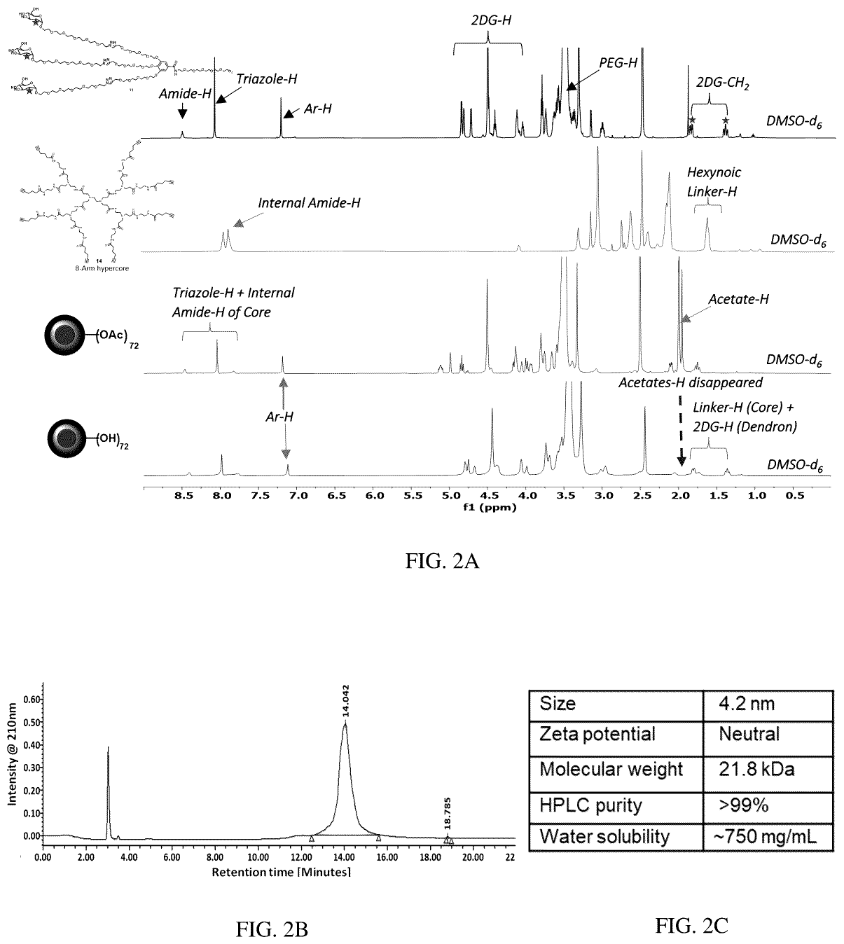

The 2DG-D synthesis was initiated with the preparation of a clickable G2 dendron (FIG. 1A). Methyl gallate (1) was alkylated at three hydroxyls with propargyltetraethyleneglycol-4-methylbenzenesulfonate (2) to afford tri-alkynated compound (3). The ¹H NMR analysis clearly showed the characteristic alkyne signals (3H) at & 2.43 ppm. This was followed by the hydrolysis with lithium hydroxide to produce compound (4) in 80% overall yield. On the other hand, peracetylated 2-deoxy-D-glucose (5) was modified with a short PEG linker with azide focal point. This involved the glycosylation of 2DG with azido-HEG-alcohol (6) in presence of Lewis acid BF₃.OEt₃, resulting in compound (7) as α-anomer (α_{D}²⁰=53.8° (c=1, CHC13). The resultant compound (7) was then clicked to the three acetylene branches of gallic acid monomer (4) via CuAAC reaction under microwave irradiations to yield compound (8). The disappearance of acetylene protons and the appearance of triazole (3H) protons at 67 7.69-7.75 ppm confirmed the completion of click reaction. Furthermore, the proton NMR spectrum also revealed the presence of acetate protons (27H) linked to three 2DG sugars, exhibiting chemical shifts within the range of δ 1.96-2.03 ppm. The carboxylic acid group on compound (8) was then reacted with azido-PEG-5-amine (9) using EDC-HOBt promoted amide coupling to afford acetyl-protected dendron (10). Appearance of characteristic amide proton (NH) at 8 8.46 ppm as well as additional PEG protons in the NMR confirmed the structure of compound 10. Subsequently, de-O-acetylation of protected dendron (10) under Zemplén transesterification conditions (NaOMe, MeOH) provided the final deprotected 2DG azide dendron (11) in quantitative yield (FIG. 1A). The ¹H NMR clearly showed the disappearance of acetate protons (FIG. 2A).

Next, we synthesized the core based on PAMAM generation-1 amine dendrimer (12) which carried 8 amine functional groups on the periphery. It was reacted with the 5-hexynoic acid (13) in the presence of amide coupling reagents to produce 8-armed acetylene functionalized core (14) in 80% yield (FIG. 1B). The complete per-NH₂-acetylene insertion at all eight terminals was confirmed by ¹H NMR analysis, which showed the characteristic methylene (—CH₂) protons (16H) of attached hexynoic acid linker at δ 1.62-1.69 ppm and additional new amide protons (8—NH—) along with internal amides (12—NH—) of G1 PAMAM dendrimer at 7.61-8.21 ppm (FIG. 2A).

Finally, the synthesis of the 2DG-D was carried out through two separate synthetic methods, one involving protected 2DG-G2 azide dendron (10) and the other using unprotected 2DG-G2 azide dendron (11) as depicted in FIG. 1B. To construct the dendrimer via the protected route, the CuAAC click reaction was performed between the octa-alkyne G1 PAMAM dendrimer core (14) and the peracetylated 2DG-azide (10) using classical click reagents, a catalytic amount of copper sulfate pentahydrate (5 mol % per alkyne), and sodium ascorbate (10 mol % per alkyne). The rection was carried out under microwave (MW) irradiation at 40° C. for 10 h to achieve 2DG-OAc dendrimer 15 (FIG. 1B) in excellent yield. The appearance of new triazole (8H) protons at 87.7 ppm confirmed the completion of click. In addition, the number of acetate protons (216H) at δ 1.93-2.01 ppm corresponds to the attachment of 24-2DG units further confirming the desired structure (FIG. 2A). The deprotection of acetate groups was achieved via Zemplén transesterification (NaOMe in methanol) to yield the final dendrimer 2DG-D (16) with 72-OH in 95% yield. The disappearance of acetate peaks in the proton NMR confirmed the formation of the deprotected product (FIG. 2A). We used protected route to confirm the successful participation of all acetylene branches in the click reaction through the presence of acetate protons in the NMR which made characterization easier and allowed us to observe any defects or missing arms in the final dendrimer. Within the ¹H NMR spectra, the acetyl protons associated with eight dendrons of 2DG were evident as separate, non-overlapping peaks, distinct from the other protons present in the macromolecule. These characteristic peaks offered a precise way to confirm the successful completion of the reaction. The same deprotected dendrimer 16 was synthesized by the reaction of PAMAM core (14) with unprotected 2DG-azide dendron (11) via click reaction at 80° C. under MW irradiation in a 95% yield. The 2DG-D was purified through tangential flow filtration (TFF) utilizing a 3 kDa cassette. As per the HPLC data, the final dendrimer displayed a purity level of >99% (FIG. 2B).

The complete structure of 2DG-D is shown in FIG. 1C. The physicochemical properties of 2DG-D are presented in FIG. 2C. The 2DG-D exhibits high water solubility (750 mg/mL) which limits the need of excipients for systemic formulations. The size of 2DG-D is 4.2 nm which allows the clearance from off-target organs through renal filtration. The 2DG-D exhibits neutral zeta potential which is important for the movement of dendrimer within the brain tissue parenchyma. Both the protected and unprotected synthetic approaches resulted in the production of 2DG-D with excellent yields, high purity, and consistent reproducibility. All intermediates and the final dendrimer were characterized via NMR and mass spectroscopy. The synthetic approach employed here doesn't necessitate the use of excessive chemicals or reagents as is often required in traditional dendrimer synthesis.

Commercially available and most widely used PAMAM dendrimers are typically manufactured using large amounts of reactants, which still frequently results in structural defects at higher generations due to sluggishness of the chemical reactions at multiple ends. On the contrary, this entire synthetic process presented here is green, reproducible, and cost-effective, making it well-suited for commercial applications.

### Reproducibility of 2DG-D Synthesis

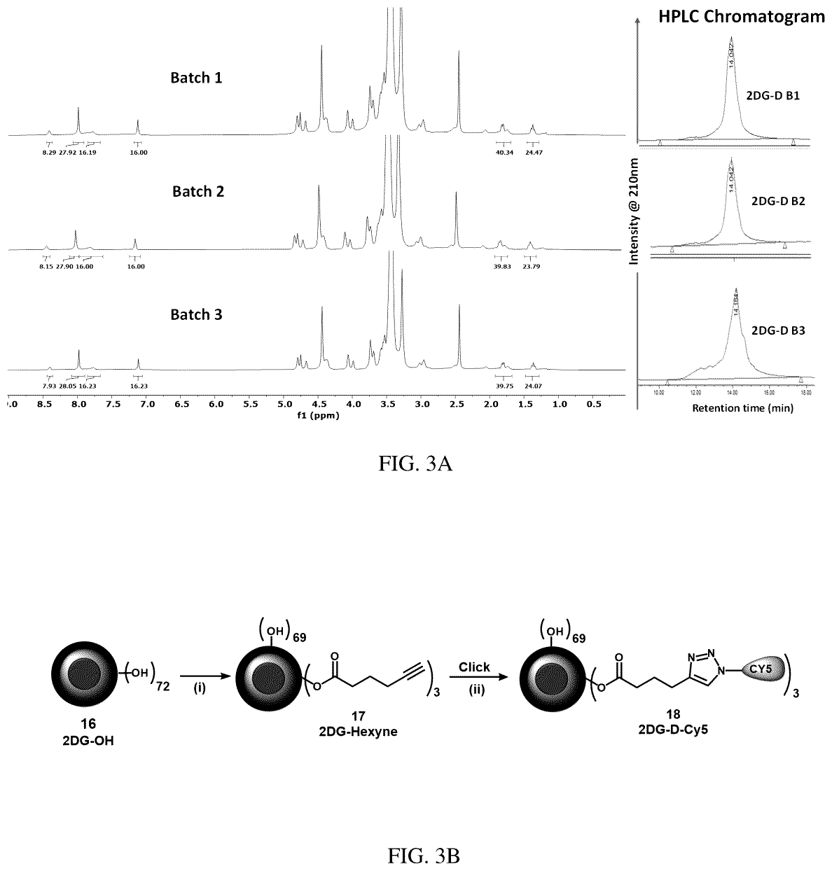

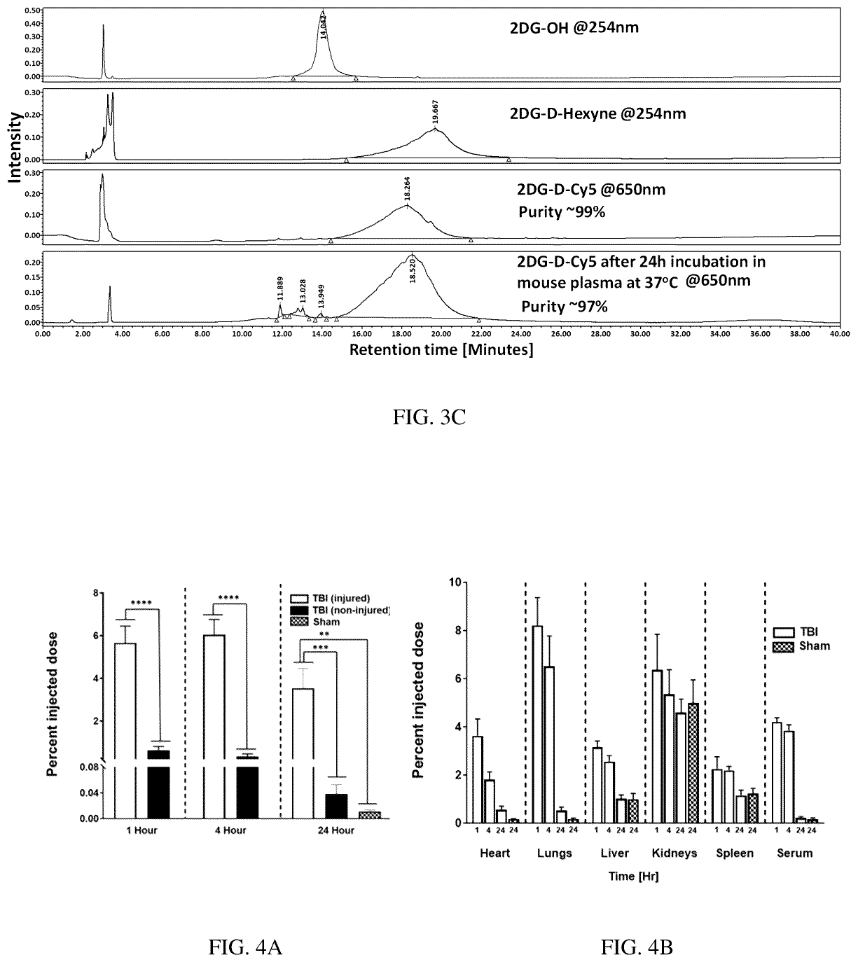

The major hurdle in the clinical translation of nanoparticle-based therapeutics is the lack of reproducibility and scalability of their synthesis process. To address these issues, we synthesized 2DG-D using a simple, convenient, and expedited synthetic strategy that allowed the precise characterization of intermediates at each step as demonstrated above. To further validate the reproducibility in the synthesis of 2DG-D, we constructed several 5 g-scale batches of 2DG-D and compared their ¹H NMRs and purity by HPLC. ¹H NMR spectra of three different batches (FIG. 3A) clearly depicted the presence of 24-2DG molecules on the surface, showing consistency in the synthesis of 2DG-D. The HPLC chromatogram of these three batches of 2DG-D showed corresponding peaks at same retention time (14 minutes), confirming the consistency in the purity of these batches (FIG. 3A).

### Fluorescent Labeling of 2DG-D

To study the in vivo brain cell uptake and organ biodistribution of 2DG-D via confocal microscopy and fluorescence spectroscopy, we introduced a near-infrared dye cyanine 5 (Cy5) at the surface of 2DG-D. We modified ˜3 hydroxyl groups on the surface of 2DG-D by reacting with 5-hexynoic acid in the presence of EDC-HOBt to afford compound (17) as illustrated in FIG. 3B. The ¹H NMR showed the presence of the additional methylene protons corresponding to hexynoic linker in the aliphatic region of dendrimer. The HPLC chromatogram showed a shift in retention time from 14 minutes for 2DG-D to 19.6 minutes for DG-D-Hexyne (17). The 2DG-D-Hexyne with acetylene groups on the surface was reacted with azide-terminating Cy5 using CuAAC reaction to obtain fluorescently labeled 2DG-D-Cy5 (18). The success of Cy5 attachment was confirmed by the presence of Cy5 protons in the ¹H NMR spectrum (FIG. 3C). The number of attached Cy5 molecules on the dendrimer surface was calculated using the proton integration method, suggesting the attachment of ˜3 molecules of the Cy5. The purity of 2DG-D-Cy5 was >98% by HPLC, and the HPLC chromatogram showed a clear shift in the retention time from 19.6 to 18.2 min upon conjugation of Cy5 (FIG. 3C). The 2DG-D was designed to target and deliver drugs intracellularly to neurons. However, the dendrimer platform itself was designed to be non-biodegradable using non-cleavable linkages in the backbone. The fluorescent 2DG-D dendrimers were stable at physiological conditions when incubated in plasma at 37° C. for 24 h (FIG. 3C) making them suitable for in vivo uptake and biodistribution studies.

### Mechanism of 2DG-D Uptake by the Neuronal Cells.

Before initiating in vitro uptake studies, we first evaluated the toxicity of the 2DG-D in cortical neurons, CATH.a neuronal cells, and RAW Blue macrophages. The cells were cultured with a gradient of 2DG-D concentrations up to 1000 μg/mL. The 2DG-D dendrimer was found to be nontoxic to neurons and macrophages as determined by Two-way ANOVA (Turkey's multiple comparisons test), with no significant effect of treatments at all the concentrations, showing no signs of toxicity. Following this, we evaluated the dose-dependent uptake of 2DG-D-Cy5 by primary cortical neurons. The microscopy based qualitative uptake results suggested that there was a concentration dependent uptake of the 2DG-D-Cy5 by the cortical neurons, when increased from ˜3.13 μg/mL to 100 μg/mL, however, the uptake tended to be saturated when the dendrimer concentration reached above 100 μg/mL. Hence, 100 μg/mL 2DG-D-Cy5 was considered optimal concentration and chosen for the further inhibitor-based uptake studies. GLUT3 is the key transporter protein that is involved in the neuronal uptake of glucose. To decipher the actual mechanism for the dendrimer uptake by both primary and secondary neurons in the present study, intracellular trafficking inhibitor based study using GLUT and other inhibitors was carried out (data not shown). From the results, it was observed that, in both cortical and CATH.a neurons, the MBCD treated cells did not show any qualitative reduction in the uptake of 2DG-D-Cy5, which was comparable to the control cells (no inhibitor) even after the inhibition of caveolae dependent endocytic pathway. In contrast to this, when cytochalasin B was used as an inhibitor for GLUT transporters, there was negligible 2DG-D-Cy5 signal in both primary and secondary neuronal cells, depicting GLUT receptors as the possible internalization pathway. Further, treatment with CPZ, a clathrin endocytosis inhibitor had no significant impact on the uptake, which was quite comparable to control. The results obtained from cytochalasin B treatment were further confirmed with phloretin, a broad-spectrum GLUT inhibitor. Similar to the results obtained with cytochalasin B, phloretin treated cells showed negligible 2DG-D-Cy5 internalization in both the neuronal cell types, which further confirmed that GLUT receptors were possibly the key players in the neuronal uptake of 2DG-D. The quantitative results obtained from the ImageJ analysis of the confocal micrographs revealed that in cortical neurons, the normalized MFI of control (˜1), was almost similar to MBCD (˜1) and CPZ (˜1.03), however in comparison to this, the normalized MFI for cytochalasin B and phloretin were ˜0.002 and ˜0.006, which suggested that for all the non-inhibited samples, the MFI was close to 1 and for the inhibited ones, it was close to zero. Similar results were observed in the case of CATH.a neurons, with normalized MFI values viz., ˜1 (control), ˜0.89 (MBCD), ˜0.93 (CPZ), ˜0.046 (Cytochalasin B), and ˜0.0001 for Phloretin. Hence from the quantitative results, it was clear that both primary and secondary neurons showed negligible uptake when incubated with GLUT inhibitors.

### Qualitative and Quantitative Brain and Organ Uptake of Systemically Administered Fluorescently 2DG-D in a Pediatric Mouse Model of TBI

We further investigated the in vivo brain and organ uptake and biodistribution of fluorescently labeled 2DG-D in a pediatric mouse model of TBI. This impact acceleration TBI model replicates the pathophysiology that is commonly observed in humans caused by falls and reliably induces diffuse axonal injury in the absence of skull fractures and parenchymal focal lesions. To evaluate the cellular co-localization of dendrimer across the BBB at the site of injury in the brain, fluorescently labeled 2DG-D conjugates were administered at 6-h post-injury and animals were euthanized at 24-h post-injection. The 6-h time point was chosen to mimic clinical situations. We found that 2DG-D was co-localized with neurons in the injured brain region. This indicates that the novel 2DG-D can cross the BBB and achieve cell-specific localization in the brain of TBI mice. We did not observe such uptake in the healthy regions of the brain of TBI animals. Since apoptotic cell death of neurons is the major hallmark of TBI, targeting these cells and rescuing them at the site of brain injury can lead to potential neurotherapeutics.

We further investigated the quantitative distribution of 2DG-D-Cy5 in the brains and organs of TBI animals at three distinct time intervals (1, 4, and 24 h; n=6-7) and compared it to age-matched sham animals (n=6). We divided the brain of the TBI animals into injured and non-injured regions to quantify the region-specific uptake of 2DG-D-Cy5. We perfused the animals with phosphate-buffered saline (PBS) to mitigate interference from blood and dendrimer lodged in blood vessels. Upon one way-ANOVA analysis, there was significant difference in the 2DG-D-Cy5 uptake (F=23.18, p<0.0001). Specifically, 1) 2DG-D-Cy5 uptake was significantly higher in the injured brain regions in the TBI animals at 1-, 4- and 24-h post-injection, compared with the shams at 24-h post-2DG-D-Cy5 administration (p<0.05), which indicates higher uptake of 2DG-D-Cy5 in injured animals, compared to healthy animals. 2) 2DG-D-Cy5 uptake significantly increased in the injured brain regions, compared with the non-injured brain regions of the TBI animals at 1-, 4-, and 24-h post-2DG-D-Cy5 administration (p<0.0001), which indicates targeted delivery of the 2DG-D-Cy5 to the injured brain region, not the non-injured brain regions. 3) There was no significant difference in the 2DG-D-Cy5 uptake at the injured brain regions at 1-, 4-, and 24-h post-2DG-D-Cy5 administration (p>0.05), which indicates the long-lasting sustained retention of the 2DG-D-Cy5 at the injured brain regions in the TBI animals. 4) There was no significant difference in the 2DG-D-Cy5 uptake between the sham animals and the non-injured brain regions of the TBI animals at 1-, 4-, and 24-h post-2DG-D-Cy5 administration (p>0.05). This implies that 2DG-D-Cy5 is not taken-up and/or retained by non-injured or healthy brain tissues even shortly after injection, indicating fast clearance, leading to less unwanted side effects (FIG. 4A). This specific uptake is significant while delivering therapies to the affected areas in the brain without affecting the healthy areas.

Next, we assessed the quantitative biodistribution of 2DG-D in other organs (FIG. 4B). Because the off-target accumulation of nanoparticles is a major concern in their clinical translation, we designed the 2DG-D with a size in the range of renal filtration to avoid any unwanted accumulation in any major organs. The biodistribution of 2DG-D was assessed in all major organs (heart, lungs, liver, kidneys, and spleen) and serum. Upon one way-ANOVA analysis, there was no significant difference in 2DG-D-Cy5 uptake in the kidneys between the TBI and sham animals at 1, 4 and 24-h post-injection (F=0.5588, p=0.6408). The presence of dendrimer in kidneys was due to the renal clearance mechanism. There was significant difference in the 2DG-D-Cy5 uptake in heart (F=16.41, p<0.0001), lungs (F=25.19, p<0.0001), liver (F=23.87, p<0.0001), spleen (F=6.192, p=0.0035), and serum (F=225.4, p<0.0001). Specifically, 2DG-D-Cy5 level in the heart, lungs, liver, spleen, and serum of the TBI animals decreased at 24 h. The clearance of dendrimer from sham animals exhibited a similar trend at 24 h, and there was no significant difference in the 2DG-D-Cy5 level at 24 h post-injection between the sham and TBI animals. Less than 0.2% dendrimer was detected in the serum 24 h after injection for both TBI and Sham animals. The results indicate that there was a temporary accumulation of dendrimer in these organs, but it did not retain and cleared by 24 h time point. The clearance via kidneys has been extensively validated for similarly sized clinical stage PAMAM-OH (4 nm, neutral) dendrimers, and other similarly sized hydroxyl dendrimers. [4, 27, 59] The precise brain uptake and retention coupled with the rapid clearance from off-target organs makes 2DG-D a unique nanoplatform for developing neuron-targeted therapies for CNS disorders.

### Synthesis and Characterization of Dendrimer-Pioglitazone (2DG-D-Pio) Conjugates

While originally approved for the treatment of non-insulin-dependent diabetes mellitus, thiazolidinediones or glitazones have shown promising neuroprotective effects in different CNS injury models. Pio is a selective agonist of the PPARγ that has shown beneficial effects in the treatment of neuronal injury and inflammation following brain injury. However, to achieve these effects, high drug levels at the target site are essential. Pio exhibits very low water solubility (0.00442 mg/ml) and low brain penetration. To achieve minimum therapeutic level concentration an increased dose is required which may lead to hypoglycemia and other severe side effects like bone loss, edema, blood cell loss, and hepatotoxicity. 2DG-D mediated targeted intracellular delivery of Pio can 1) decrease the dose and systemic side effects, 2) enhance the efficacy to attenuate the ongoing neuronal injury and inflammation, and 3) improve the aqueous solubility by several folds.

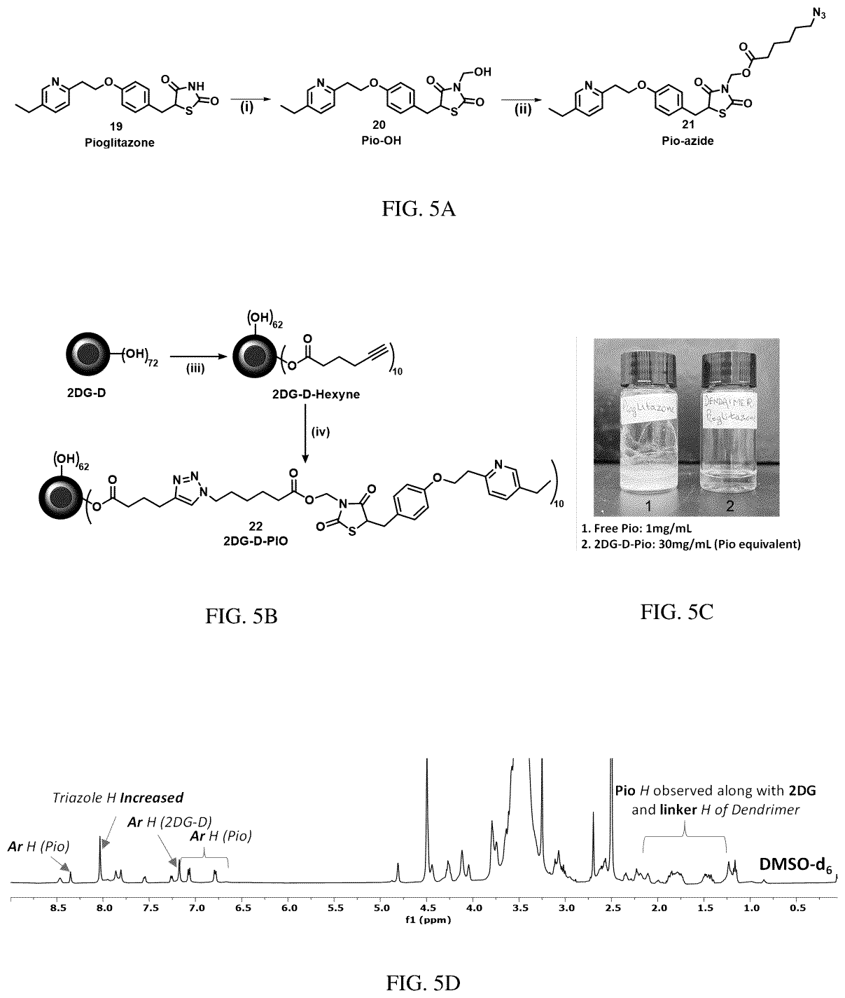

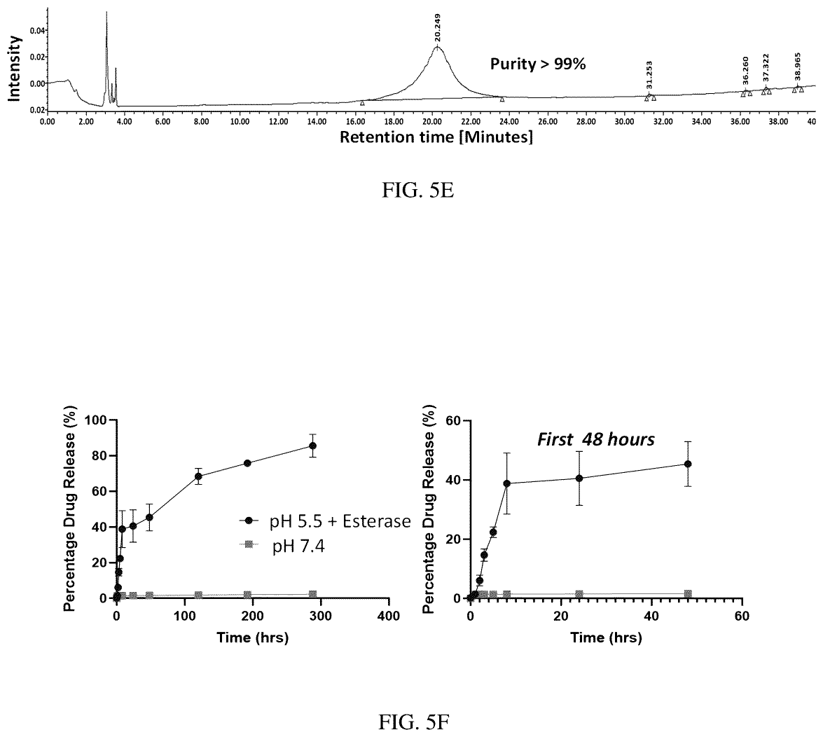

To obtain the 2DG-D-Pio conjugate, Pio containing a clickable linker (azide) was first synthesized. Pio (19) was treated with formaldehyde to produce Pio-OH (20), which was subsequently reacted with azido-hexynoic acid in the presence of EDC-DMAP to yield Pio-azide (21) as depicted in FIG. 5A. On the other hand, 2DG-D was modified to bring ˜10 acetylene arms, which were reacted with pio-azide (21) using CuAAC click reaction to obtain 2DG-D-Pio dendrimer conjugate 22 (FIG. 5B). After dialysis purification and freeze-drying, the 2DG-D-Pio was obtained in 95% yield. ¹H NMR confirmed the attachment of 10 drug molecules per dendrimer corresponding to ˜12 weight percent drug loading. (FIG. 5D). The characterization of all the intermediates and final conjugates was carried out by NMR and mass spectroscopy. The purity of 2DG-D-Pio was ˜99% by HPLC (FIG. 5E). The 2DG-D-Pio exhibited remarkable solubility in water exceeding 30 mg/mL (FIG. 5C). This represents a substantial improvement in the solubility of Pio when conjugated to 2DG-D while the free drug exhibits negligible water solubility (0.00442 mg/ml). The dendrimer conjugation remarkably improved the Pio water solubility by ˜6000 fold.

We further evaluated the shelf stability of 2DG-D-Pio formulation in PBS. The stability was assessed both at room temperature (RT) and 4° C., over a period of 28 days. Remarkably, even after 28 days of storage, both at 4° C. and RT, the 2DG-D-Pio formulations retained their stability, with purities of 99.34% and 98.24%, respectively as determined by HPLC. Importantly, there were no shifts in retention time observed during the entire 28-day period with no detectable release of Pio from the formulation under these storage conditions suggesting the stability of the conjugate.

Next, we conducted the in vitro drug release study from the conjugate, both in conditions mimicking the extracellular environment (physiological pH, PBS buffer at pH 7.4) and intracellular conditions under the influence of an enzyme (carboxyl-esterase, pH 5.5) (FIG. 5F). In PBS buffer at pH 7.4, we detected negligible release of the drug, indicating the plasma stability of the 2DG-D-Pio conjugate. To mimic the in vivo conditions, we further evaluated the stability of 2DG-D-Pio in serum supported PBS. The 2DG-D-Pio did not show any signs of degradation or Pio release over a period of 7 days as demonstrated by a HPLC chromatogram at various time points. This is important to maintain the target-specific intracellular delivery of the drug. At intracellular conditions, the dendrimer-drug conjugate exhibited a gradual and sustained release of the free drug. Over a 48 h period, less than 50% of the drug was released, and this release continued, reaching approximately 80% drug release over a 12-day period (FIG. 5F).

### Preliminary Biosafety Studies.

Hemocompatibility studies. Before in vivo efficacy evaluation of the 2DG-D and 2DG-D-Pio dendrimers, ex vivo hemocompatibility studies were carried out to ensure that the dendrimers were safe to use under in vivo settings. From the hemolysis studies, it was observed that at all tested concentrations, the dendrimers were showing excellent compatibility with the rat RBCs. The UV absorption studies suggested no significant peak intensity at 540 nm for all tested concentrations, except for Triton X-100 control, suggesting no signs of hemolysis (data not shown). Further, the quantitative analysis suggested that the percentage hemolysis at each dendrimer concentration for 2DG-D was ˜4% (5 mg/ml), ˜3.7% (2.5 mg/ml), ˜4.1% (1.25 mg/ml), ˜3.9% (0.63 mg/ml), and ˜4.1% (0.31 mg/ml) and for 2DG-D-Pio, it was, ˜4.1% (5 mg/ml), ˜4.0% (2.5 mg/ml), ˜3.9% (1.25 mg/ml), ˜3.8% (0.63 mg/ml), and ˜3.9% (0.31 mg/ml). The overall hemolysis was found to be less than 5% for all the samples. Hence, it can be inferred from the results that, there are no toxic and hemolytic effects of 2DG-D and 2DG-D-Pio on the RBCs, suggesting safe use of both these dendrimers for in vivo efficacy evaluation.

In vivo biosafety studies. To further assess the systemic toxic effects of 2DG and 2DG-D-Pio, 100 μL of 2DG-D-Pio (5 mg/kg) or equivalent amount of 2DG-D was administered, while the control group received the same volume of saline. No weight loss was observed after 3 days of treatment (data not shown). Liver enzymes, including alanine aminotransferase (ALT) and aminotransferase (AST), renal function index, creatinine (CRE), and urea nitrogen (BUN) were tested, and the levels were within the normal range in both the treatment and control groups (data not shown). In addition, there was no significant damage or difference in liver and kidney sections stained with hematoxylin-eosin after 3 days in the control and dendrimer-treated groups (data not shown). These data suggest that dendrimers are not toxic to the liver and kidney of male and female mice.

### 2DG-D-Pio Improved Behavioral Outcomes

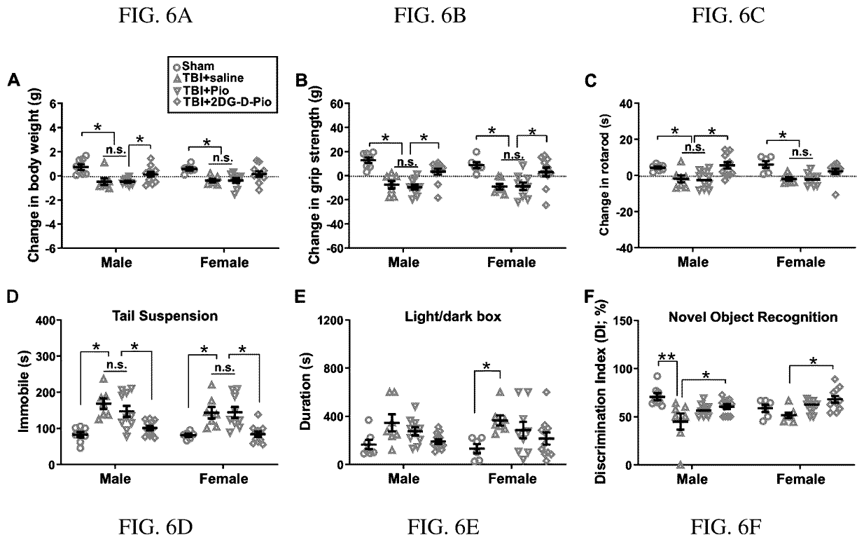

To evaluate the efficacy of Pio and 2DG-D-Pio on body weight, we compared the change in the body weight before TBI (baseline) and at 1-d post-treatment. Upon two-way ANOVA analysis [sex (male, female), treatment (sham, TBI+saline, TBI+Pio, TBI+2DG-D-Pio], there were significant differences in the (body weight)_{change} based on treatment [F_{(3,62)}=12.58, p<0.0001]. Specifically, body weight significantly decreased in both male and female TBI+saline and TBI+Pio groups, compared with the male (p<0.01) and female sham groups (p<0.05). Moreover, body weight significantly decreased in the male TBI+Pio group, compared with the TBI+2DG-D-Pio group (p<0.05) (FIG. 6A). To evaluate the muscle strength and sensorimotor coordination, we compared the changes in the grip strength and rotarod performance before (baseline) and at 1-d post-treatment. For grip strength, upon two-way ANOVA analysis, there were significant differences based on treatment [F_{(3, 62)}=19.89, p<0.0001]. Specifically, grip strength significantly decreased in both male and female TBI+saline (p<0.001) and TBI+Pio (p<0.05) groups, compared with the male and female sham and TBI+2DG-D-Pio groups (FIG. 6B). For the Rotarod test, upon two-way ANOVA analysis, there were significant differences based on treatment [F_{(3, 62)}=12.90, p<0.0001]. Specifically, the latency to the first fall significantly decreased in both male and female TBI+saline (p<0.05) and TBI+Pio (p<0.05) groups, compared with the male and female sham groups. In addition, the latency to the first fall significantly decreased in the male TBI+saline (p<0.05) and TBI+Pio (p<0.05) groups, compared with the male TBI+2DG-D-Pio group (FIG. 6C). Next, tail suspension and light/dark box tests were used to evaluate the anxiety and depression-like behaviors at 1-d post-treatment. For the tail suspension test, upon two-way ANOVA analysis, there were significant differences based on treatment [F_{(3, 62)}=19.27, p<0.0001]. Specifically, the duration of immobile significantly increased in both male and female TBI+saline (p<0.01) and TBI+Pio (p<0.01) groups, compared with the male and female sham and TBI+2DG-D-Pio groups (FIG. 6D). For the light/dark box test, upon two-way ANOVA analysis, there were significant differences in the time spent in the light compartment based on treatment [F_{(3, 62)}=6.29, p=0.0009]. Specifically, the duration significantly increased in the female TBI+Pio group, compared with the female sham group (p<0.05) (FIG. 6E). Novel object recognition test was used to evaluate cognitive function at 1-d post-treatments. Upon two-way ANOVA analysis, there were significant differences in the discrimination index based on treatment [F_{(3, 62)}=7.59, p=0.0002]. Specifically, the mice in the male TBI+saline group spent significantly less time with the novel object, compared with the male sham (p<0.001) and TBI+2DG-D-Pio (p<0.05) groups. the mice in the female TBI+saline group spent significantly less time with the novel object, compared with the female TBI+2DG-D-Pio (p<0.05) groups (FIG. 6F).

In vivo results demonstrated that 2DG-D-Pio showed a better efficacy in improving behavioral outcomes, compared with the free drug. For example, the body weight, grip strength and Rotarod performance significantly decreased in both male and female saline-treated and Pio-treated TBI animals, but not in the 2DG-D-Pio treated group. Moreover, 2DG-D-Pio treatment significantly improved body weight, grip strength and Rotarod, compared with free Pio treatment in males. In females, 2DG-D-Pio treatment significantly improved grip strength, compared with free Pio treatment. In addition, 2DG-D-Pio treatment significantly decreased immobile time during tail suspension test, compared with the TBI+saline and TBI+Pio groups in both males and females. Whereas Pio-treated TBI animals did not show significant improvement.

### 2DG-D-Pio Improved Neuroinflammatory Responses

We first evaluated the effects of 2DG-D alone on neuroinflammation and cell death. TBI mice received intraperitoneal injection of 2DG-D (100 μL) or saline (100 μL) at 6-h post-injury. Animals were euthanized at 24-h post-treatment. The neurons from the injured brain regions were isolated for gene expression evaluation. We found that there was no significant difference in the expression of neuroinflammatory and cell death markers between the TBI+saline and TBI+2DG-D groups. These data indicate that 2DG-D alone did not have significant effect on neuroinflammation and cell death. Next, we evaluated the efficacy of Pio and 2DG-D-Pio on neuroinflammatory responses using brain tissues from the injured brain regions (or the matching areas in the sham), which included all brain cell types (e.g. neurons, microglia, astrocytes, etc.). We found that 2DG-D-Pio significantly improved neuroinflammatory responses, compared with the TBI+saline mice. 2DG-D-Pio also showed a better efficacy than free Pio but did not reach statistical significance.

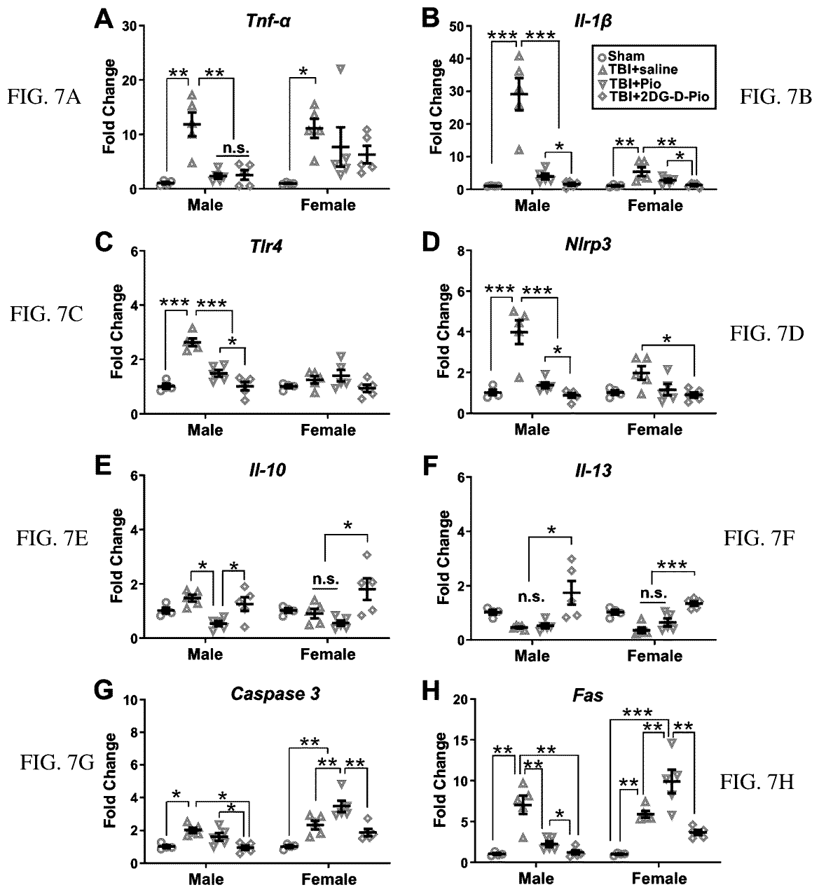

To evaluate the effect of treatment on neurons, we isolated primary neurons from the injured brain regions of TBI animals (or the matching areas in the sham) after treatment to further evaluate the efficacy of Pio and 2DG-D-Pio on neuro-inflammatory responses and cell death specifically in neurons. We first compared the mRNA expression of pro-inflammatory markers (TNF-α, IL-1β, TLR4, and NLRP3) at 1-d post-treatments. The expression of these pro-inflammatory markers was significantly higher in saline-treated TBI animals compared to sham controls. The TNF-α expression significantly decreased in TBI male mice treated with free Pio (p<0.05) or 2DG-D-Pio (p<0.05) compared to saline-treated TBI animals (FIG. 7A). More specifically, upon two-way ANOVA analysis [sex (male, female), treatment (sham, TBI+saline, TBI+Pio, TBI+2DG-D-Pio)], there were significant differences in the TNF-α expression based on treatment [F_{(3, 30)}=10.84, p<0.0001]. Specifically, TNF-α expression significantly increased in both male (p<0.01) and female (p<0.01) TBI+saline group, compared with the male and female sham groups. Moreover, TNF-α expression significantly increased in the male TBI+saline group, compared with the male TBI+Pio (p<0.05) and TBI+2DG-D-Pio (p<0.05) groups (FIG. 7A).

We further evaluated the IL-1 B expression. IL-1β increases early following experimental and human TBI, and is closely associated with injury severity. It stimulates glutamate excitotoxicity and promotes cell loss, [66] while neutralization of IL-1ß reduces neuronal death and improves cognitive outcome after TBI. There were significant differences in the IL-1β expression based on treatment [F_{(3, 30)}=31.66, p<0.0001], sex [F_{(3, 30)}=20.31, p<0.0001], and the interaction (treatment x sex) [F_{(3, 30)}=17.99, p<0.0001]. In males, IL-1β expression significantly increased in the TBI+saline group, compared with sham (p<0.0001), TBI+Pio (p<0.0001) and TBI+2DG-D-Pio (p<0.0001) groups. Moreover, IL-1ß expression significantly increased in the TBI+Pio group, compared with the TBI+2DG-D-Pio group (p<0.05). In females, IL-1β expression significantly decreased in 2DG-D-Pio treated TBI animals compared to saline-treated animals (p<0.001) groups and this reduction was significantly more in 2DG-D-Pio group when compared to the free Pio group (p<0.05) (FIG. 7B), suggesting the effect of neuron targeted delivery of Pio via 2DG-D-Pio.

We next examined the effect on TLR4 expression. There were significant differences in the TLR4 expression based on treatment [F_{(3, 30)}=19.4, p<0.0001], sex [F_{(3, 30)}=13.87, p=0.0008] and the interaction (treatment x sex) [F_{(3, 30)}=10.44, p<0.0001]. Specifically, TLR4 expression significantly increased in the male TBI+saline group, compared with the male sham (p<0.0001). The TLR4 expression decreased significantly in both TBI+Pio (p<0.0001), and TBI+2DG-D-Pio (p<0.0001) groups compared to saline treated TBI males. The dendrimer conjugate 2DG-D-Pio had a better effect in reducing the TLR4 expression compared to free Pio (p<0.05) groups (FIG. 7C). There were significant differences in the NLRP3 expression based on treatment [F_{(3, 30)}=23.21, p<0.0001], sex [F_{(3, 30)}=7.2, p=0.0117] and the interaction (treatment x sex) [F_{(3, 30)}=5.8, p=0.0030]. A similar trend to TLR4 expression was found in NLRP3 expression in males. However, the 2DG-D-Pio treated female TBI animals showed significantly reduced NLRP3 expression compared to the TBI+Pio (p<0.05) group (FIG. 7D).

Next, we compared the mRNA expression of anti-inflammatory markers (IL-10 and IL-13) at 1-d post-treatments. IL-10 is an anti-inflammatory cytokine that can inhibit the expression of pro-inflammatory factors and mediate the recovery process following TBI. Moreover, IL-10 can prevent prolonged secondary brain damage by facilitating cytokine storm resolution. Upon two-way ANOVA analysis, there were significant differences in the IL-10 expression based on treatment [F_{(3, 30)}=8.33, p=0.0004]. Specifically, in males, IL-10 expression significantly decreased in the TBI+Pio group, compared with the TBI+saline (p<0.05) and TBI+2DG-D-Pio (p<0.01) groups. In females, IL-10 expression significantly increased in the TBI+2DG-D-Pio group, compared with the TBI+saline (p<0.05) and TBI+Pio (p<0.01) groups (FIG. 7E). We next examined the IL-13 expression. IL-13, an anti-inflammatory cytokine, plays an important role in learning and memory. IL-13 is expressed in neurons and can modulate neuronal activity and synaptic plasticity. Studies have shown that IL-13 protects neurons against excitotoxic insults after TBI. There were significant differences in the IL-13 expression based on treatment [F_{(3, 30)}=15.41, p<0.0001]. IL-13 expression significantly increased in both male (p<0.05) and female (p<0.001) TBI+2DG-D-Pio groups, compared with the male and female TBI+saline and TBI+Pio groups (FIG. 7F). In our previous studies, we have demonstrated that pediatric TBI causes aberrant neuro-inflammatory responses, abnormal protein accumulation, and cell loss, resulting in impaired sensorimotor function, cognitive deficits, and depressive behaviors. Therefore, the decreased IL-1β and increased IL-10 and IL-13 after 2DG-D-Pio treatment might rescue neuronal loss and be responsible for the improved neuro-behaviors.

We further compared the mRNA expression of cell death markers (Caspase-3 and Fas) at 1-d post-treatments. Upon two-way ANOVA analysis, there were significant differences in the caspase-3 expression based on treatment [F_{(3, 30)}=19.08, p<0.0001], sex [F_{(3, 30)}=24.27, p<0.0001], and the interaction (treatment x sex) [F_{(3, 30)}=6.80, p=0.0012]. In both males and females, the Caspase 3 expression significantly increased in saline treated TBI animals compared to sham controls. In males, both free Pio and 2DG-D-Pio treatment decreased the Caspase 3 expression compared to saline-treated group, however, the expression significantly decreased in the TBI+2DG-D-Pio group compared to the free Pio group (p<0.05). In females, while 2DG-D-Pio treatment significantly lowered the Caspase 3 expression compared to the saline-treated animals, the treatment with free Pio showed an increase in the expression of Caspase 3 (FIG. 7G). We further evaluated the Fas expression, another cell death marker. There were significant differences in the Fas expression based on treatment [F_{(3, 30)}=26.24, p<0.0001], sex [F_{(3, 30)}=18.66, p=0.0002], and the interaction (treatment x sex) [F_{(3, 30)}=14.72, p<0.0001]. Similar to Caspase 3, the Fas expression was significantly higher in saline treated TBI animals compared to sham controls irrespective of the sex. In males, both dendrimer conjugate and free drug treated animals showed significantly reduced Fas expression compared to saline treated animals, however, the reduction was significantly more in the 2DG-D-Pio group compared to the free Pio treated animals. Like the trend seen in females for Caspase 3 expression, the free Pio treated animals demonstrated significantly higher Fas expression compared to sham control, saline-treated, and 2DG-D-Pio treated animals. In females, Fas expression significantly increased in the TBI+Pio group, compared with the TBI+saline (p<0.01) and TBI+2DG-D-Pio (p<0.01) groups (FIG. 7H).

Previous studies have shown that Pio has neuroprotective and anti-inflammatory effects after TBI. For example, in a controlled cortical impact (CCI) model of TBI, Pio treatment at 15 min post-injury exhibits neuroprotective function via activating PPARγ and reducing NF-κB and IL-6. In the present study, we chose the 6 h time point to reflect the clinical treatment time for TBI patients, which provides the basis for assessment of therapeutic time window of the novel dendrimer platform. Interestingly, there are sex differences in the behavioral outcomes and the expression of inflammatory and cell death markers among treatment groups. Studies have shown that there are sex differences in the pharmacokinetics of Pio, and the efficacy of Pio treatment in nonalcoholic fatty liver disease is also gender dependent. Moreover, growing evidence indicates that TBI alone can induce sex-specific neuroinflammatory responses and behavioral outcomes, depending on differential cellular responses, sex hormones, and metabolism. Therefore, the sex differences in behaviors and neuroinflammatory responses can be caused by the combined effects of brain injury and pioglitazone treatment, however, the underlying mechanisms need to be further investigated.

### Conclusions

The primary challenge in treating brain disorders has long been the major hurdle in drug delivery across the BBB. Even if drugs or nanoparticles manage to traverse the compromised BBB after brain injury or neuroinflammation, effectively reaching key cells involved in brain diseases, such as neurons, remains a significant hurdle. Typically, nanocarriers are modified with targeting ligands to facilitate delivery across the BBB to specific cell types. However, this post-synthetic functionalization process is often time-consuming and meticulous, posing challenges related to batch-to-batch reproducibility and other variabilities. The distinctive feature of 2DG-D is its straightforward synthesis and inherent capacity to target and localize within neurons precisely at the site of brain injury. We have successfully demonstrated the delivery of a neuroprotective drug, pioglitazone, using 2DG-D, resulting in improved behavioral outcomes and neuroinflammatory responses in a pediatric mouse model of TBI. These promising in vitro and in vivo results coupled with a simple approach for the construction of 2DG-D makes it a useful nanoplatform for addressing brain diseases.

### Example 2

Traumatic brain injury (TBI) remains a major global health challenge, characterized by high morbidity and mortality rates. Despite advances in neuroscience, the blood-brain barrier (BBB) limits the effectiveness of potential neuroprotective treatments. Recent nanotechnology breakthroughs have led to smart drug delivery systems that can cross the BBB and target injured brain areas. However, achieving the specificity needed to deliver therapies to affected neurons remains a challenge. As described above, we developed a mixed-layered dendrimer functionalized with 2-deoxyglucose (2DG-D) for selective neuronal drug delivery. In this study, we explore the therapeutic potential of rosiglitazone (Rosi) for pediatric TBI by creating a 2DG-D-Rosi nanosystem, where Rosi is conjugated to 2DG-D to improve its solubility, bioavailability, and targeted delivery to injured neurons. In vitro, 2DG-D-Rosi demonstrated high neuronal uptake, sustained drug release, and excellent biocompatibility. It significantly reduced neuronal apoptosis, ROS formation, pro-inflammatory cytokine expression, and caspase activity, outperforming free Rosi. In vivo, using a pediatric TBI mouse model, 2DG-D-Rosi improved neuronal targeting, reduced neuroinflammation, and enhanced behavioral outcomes. This research highlights 2DG-D-Rosi as a useful nanotherapeutic platform for precise TBI treatment.

### Experimental Section

### Materials

All starting materials and reagents [rosiglitazone (Rosi), formaldehyde, triethylamine (TEA), 1-[3-(dimethylamino) propyl]-3-ethylcarbodiimide (EDC), 4-(dimethylamino)pyridine (DMAP), N,N-diisopropylethylamine (DIPEA), copper sulfate pentahydrate (CuSO₄.5H₂O), and sodium ascorbate (NaAs)] were sourced from Sigma Aldrich US, Merck, or Thermo Fisher Scientific, and were used as received. Solvents were of analytical grade and used as received without additional purification. Thin-layer chromatography (TLC) was performed using silica-coated aluminum sheets with F₂₅₄ fluorescent indicator (purchased from Merck). Column chromatography was performed using silica gel 60 (70-230 mesh) as stationary phase. Spectra/Por dialysis membranes from Repligen were used for the dialysis. PBS, Poly-L-lysine, 4′,6-diamidino-2-phenylindole (DAPI), 3-(4,5-dimethylthiazol-2-yl)-2,5 diphenyltetrazolium bromide (MTT), and Triton® X-100 were procured from Aaron Chemicals. CATH.a, EOC 20 cell line was obtained from ATCC, United States. DMEM, RPMI-1640 was purchased from Gibco, FBS were obtained from Gibco Scientific and VectaShield® antifade mounting media was obtained from VectorLabs. Rat red blood cells (RBCs) were purchased from Innovative Research. TNF-α-II-6 ELISA kits were obtained from BioLegend, USA. Caspase activity stain was procured from Revvity. FITC ApoScreen® Annexin V Apoptosis Kit was procured from Southern Biotech. All these above-mentioned reagents were used as such.

### Instrumentation/Methods

NMR spectra were recorded using a Bruker 500 MHz spectrophotometer. The synthesized compounds were dissolved in appropriate deuterated solvents (CDCl₃/DMSO-d₆/D₂O) to obtain spectral data for proton NMR (¹H NMR, 500 MHz) and carbon NMR (13C NMR, 125 MHz) at 25° C. Chemical shifts (δ) for ¹H-NMR were reported in parts per million (ppm) using the solvent peak as a reference. The coupling constants (J) were expressed in hertz (Hz). The following abbreviations are used to describe the signal patterns: s (singlet), d (doublet), t (triplet), q (quartet), and m (multiplet). The molecular weights of the small molecules were determined using a Bruker micrOTOF II spectrometer with electrospray ionization (ESI) as the ionization source. For dendrimers and dendrimer conjugates, mass analysis was performed via direct infusion using ESI on a Bruker MALDI-TOF instrument employing trans-2-[3-(4-tert-butylphenyl)-2-methyl-2-propenylidene] malononitrile as the matrix.

High-performance liquid chromatography (HPLC) was performed using a Waters Acquity Arc® HPLC system equipped with binary pumps, a 2998 photodiode array detector, and a 2475 fluorescence detector. Data was analyzed using the Waters Empower software. Chromatographic separation was achieved with a Waters Symmetry C18 column (5 μm, 4.6×250 mm) using the gradient flow method at a flow rate of 1 mL/min. The mobile phase consisted of water containing 0.1% trifluoroacetic acid (Solvent A) and acetonitrile containing 0.1% trifluoroacetic acid (Solvent B). The gradient was initiated at 90:10 (A:B), adjusted to 40:60 (A:B) over 20 min, maintained at 40:60 (A:B) for 5 min, and returned to the initial ratio of 90:10 (A:B) by 45 min. The Cy5 labeled 2DG-D-Rosi was analyzed using a different gradient method. The gradient was initiated at 90:10 (A:B), adjusted to 50:50 (A:B) for over 20 min, then changed to 10:90 (A:B), and maintained for 18 min, before returning to the initial ratio of 90:10 (A:B) by 40 min. The Rosi, Rosi derivatives, 2DG-D, 2DG-D-Rosi, and their derivatives were detected at 210, 233, and 254 nm wavelengths while the Cy5 labeled dendrimer conjugate was specifically monitored at 650 nm. The HPLC column temperature was maintained at 25° C. to ensure consistent retention times and optimal peak resolutions.

The size distribution and zeta potential of the dendrimer and dendrimer-drug derivatives were analyzed using dynamic light scattering (DLS) on a Malvern Zetasizer® Nano 90 (Westborough, MA, USA). For size distribution analysis, the samples were prepared at the concentration of 0.1 mg/mL in Milli-Q® water. For zeta potential measurements, the samples were dissolved at a concentration of 0.2 mg/mL in 10 mM sodium chloride solution. All measurements were performed in triplicates to ensure accuracy.

### Synthesis Protocols

Compound 5 (2DG-D) and compound 9 (2DG-D-Hexyne with 10 alkyne arms) were synthesized using our previously published protocols.

Synthesis of Compound 2: Rosi 1 (250 mg, 0.516 mmol) was dissolved in anhydrous DMF (3 mL) and treated with formaldehyde (33.6 mg, 11.2 mmol) and triethylamine (1.6 mL 11.2 mmol). The reaction mixture was stirred in an inert environment for 10 hours, and the completion of reaction was confirmed by thin-layer chromatography (TLC). The mixture was then diluted with DCM (300 mL), and the organic layer was sequentially washed with water (20 mL×2) and brine (20 mL×2), dried with anhydrous Na₂SO₄, filtered, and evaporated in vacuo. The crude product was purified using silica gel flash column chromatography [ethyl acetate/hexane, 40:60 (v/v)] to afford compound 2 in 87% yield (off-white powder).

¹H NMR (500 MHZ, DMSO-d₆) δ 8.12-8.04 (m, 1H), 7.50 (t, J=8.1 Hz, 1H), 7.10-7.03 (m, 2H), 6.88-6.79 (m, 2H), 6.65 (d, J=8.6 Hz, 1H), 6.60-6.52 (m, 1H), 5.65 (t, J=5.6 Hz, 1H), 4.71 (d, J=7.4 Hz, 1H), 4.09 (q, J=6.0 Hz, 2H), 3.99-3.82 (m, 3H), 3.67-3.55 (m, 1H), 3.19-3.02 (m, 4H), 2.98 (d, J=13.9 Hz, 1H).

¹³C NMR (125 MHZ, DMSO-d₆) δ 176.1, 171.2, 158.5, 158.0, 148.0, 137.8, 131.9, 131.9, 127.5, 127.2, 114.5, 114.4, 112.0, 106.2, 70.1, 68.2, 66.3, 66.1, 65.7, 64.0, 48.9, 38.8, 38.6, 37.5.

ESI-MS: m/z: calculated for C19H21N3O4S: 387.13; found as [M+H]+: 388.13

Synthesis of Compound 4: In a round bottom flask, 6-azidohexanoic acid (200 mg, 0.516 mmol) was dissolved in anhydrous DMF (5 mL) and mixed with 1-Ethyl-3-(3-dimethylaminopropyl) carbodiimide (160.21 mg, 1.032 mmol) under argon atmosphere. After stirring for 3 minutes, Compound 2 (162 mg, 1.032 mmol) and 4-dimethylaminopyridine (DMAP, 56.7 mg, 0.46 mmol) were added to the reaction mixture. On completion, the reaction mixture was diluted with DCM (300 mL) and the organic layer was washed with water (20 mL×2), and brine. The organic layer was then dried using anhydrous Na₂SO₄, filtered, and evaporated under vacuum. The crude product was purified using silica flash column chromatography [Ethyl acetate/Hexane, 20:80 (v/v)] to afford compound 4 as an off-white oil in 44.7% yield.

¹H NMR (500 MHZ, DMSO-d₆) δ 8.07 (dd, J=4.9, 1.9 Hz, 1H), 7.57-7.44 (m, 1H), 7.10 (d, J=8.5 Hz, 2H), 6.95-6.82 (m, 2H), 6.63 (d, J=8.6 Hz, 1H), 6.56 (dd, J=7.0, 4.9 Hz, 1H), 4.52 (d, J=11.4 Hz, 1H), 4.36 (d, J=11.4 Hz, 1H), 4.10 (t, J=5.9 Hz, 2H), 3.88 (t, J=5.9 Hz, 2H), 3.32-3.25 (m, 3H), 3.19 (d, J=13.8 Hz, 1H), 3.10 (d, J=13.9 Hz, 1H), 3.06 (s, 3H), 2.32 (t, J=7.2 Hz, 2H), 1.63-1.43 (m, 4H), 1.38-1.21 (m, 2H).

¹³C NMR (125 MHZ, DMSO-d₆) δ 172.4, 158.5, 158.2, 148.0, 137.8, 132.1, 126.6, 114.5, 112.0, 106.2, 66.4, 65.7, 50.9, 48.9, 38.8, 37.5, 33.7, 28.3, 26.0, 24.4.

ESI-MS: m/z: calculated for C24H28N605S: 512.18; found as [M+H]⁺: 513.19.

Synthesis of 2DG-D-Hexyne (Compound 7): 5-Hexynoic acid 6 (11.5 mg, 14.0 eq, 0.098 mmol) was dissolved in dry DMF (6 mL). The coupling reagents EDC.HCl (8.35 mg, 0.078 mmol) and DMAP (6.0 mg, 0.009 mmol) were added and stirring was continued at room temperature for 20 minutes. To this reaction vessel, 2DG-D dendrimer 5 (150 mg, 1.0 eq, 0.007 mmol) in 3 mL of dry DMF was added dropwise for 20 minutes and allowed to stir for 24 hours. The progress of the reaction was monitored using HPLC. Upon completion, the reaction mixture was purified by dialysis using a 1 kDa dialysis membrane first in DMF for 16 h, followed by dialysis in deionized (DI) water for an additional 16 h. The product was then lyophilized to obtain compound 7 in 84% yield.

¹H NMR (500 MHZ, DMSO-d₆) δ 8.52 (s, 8H), 8.31-7.73 (m, 53H), 7.24 (s, 16H), 5.26-5.08 (m, 9H), 5.00-4.76 (m, 61H), 4.69-4.33 (m, 148H), 4.28-4.03 (m, 64H), 3.91-3.79 (m, 108H), 3.73-3.63 (m, 138H), 3.61-3.50 (m, 885H), 3.23-2.98 (m, 67H), 2.90-2.60 (m, 44H), 2.46 (t, J=7.2 Hz, 28H), 2.35-2.08 (m, 56H), 1.98-1.71 (m, 57H), 1.56-1.40 (m, 24H).

¹³C NMR (125 MHz, DMSO-d₆) δ 173.2, 166.0, 152.2, 144.3, 140.4, 129.7, 124.7, 106.7, 97.2, 97.1, 73.6, 72.3, 72.1, 72.1, 71.1, 70.5, 70.4, 70.3, 70.3, 70.2, 70.2, 70.1, 70.1, 70.0, 70.0, 69.5, 69.4, 69.2, 69.2, 68.8, 68.4, 68.2, 66.2, 66.0, 64.1, 64.0, 61.5, 50.9, 49.8, 38.3, 38.2, 33.8, 28.4, 26.1, 26.0, 25.6, 24.5.

Synthesis of 2DG-D-Rosi (Compound 8): A solution of 2DG-D-Hexyne 7 (50 mg, 1.0 eq, 0.0021 mmol) in DMF (0.2 mL) was added to a stirred solution of compound 4 (0.015 mg, 14 eq, 0.030 mmol) in DMF (0.1 mL) in a reaction vial. This was followed by a sequential addition of CuBr (10 mol % per acetylene, 12 mg), PMDETA (4.5 mg), and THPTA (1.5 mg, cat). The reaction mixture was then stirred at RT for 10 h. Upon completion, purification was performed using a 1 kDa dialysis membrane against DI water for 12 hours. The aqueous solution was then lyophilized to obtain compound 8 in 95% yield (brown solid).

¹H NMR (500 MHZ, DMSO-d₆) δ 8.47 (t, J=5.6 Hz, 8H), 8.13-7.98 (m, 52H), 7.86-7.79 (m, 29H), 7.48 (t, J=7.8 Hz, 15H), 7.18 (s, 16H), 7.09 (d, J=8.2 Hz, 29H), 6.85 (d, J=8.2 Hz, 28H), 6.62 (d, J=8.6 Hz, 14H), 6.54 (t, J=6.0 Hz, 14H), 4.88-4.77 (m, 32H), 4.59-4.36 (m, 180H), 4.35-4.17 (m, 90H), 4.16-3.99 (m, 141H), 3.97-3.53 (m, 685H), 3.40-3.23 (m, 191H), 3.16-2.96 (m, 178H), 2.83-2.53 (m, 119H), 2.39-2.04 (m, 146H), 1.91-1.66 (m, 115H), 1.55-1.35 (m, 64H), 1.30-1.14 (m, 47H).

¹³C NMR (125 MHZ, DMSO-d₆) δ 173.1, 172.4, 158.5, 158.1, 152.2, 148.0, 144.2, 137.8, 132.0, 124.7, 122.2, 114.4, 112.0, 106.7, 106.2, 97.2, 97.1, 73.5, 72.1, 70.4, 70.3, 70.2, 70.2, 70.2, 70.1, 70.1, 70.0, 69.4, 69.2, 69.2, 68.8, 68.4, 68.2, 66.6, 66.2, 66.0, 65.7, 63.9, 61.5, 49.7, 49.4, 48.9, 40.4, 40.4, 40.3, 40.2, 40.1, 40.0, 39.9, 39.8, 39.6, 39.4, 38.3, 37.5, 33.6, 33.4, 29.8, 25.7, 25.6, 25.1, 24.8, 24.2.

MALDI-TOF: m/z: calculated for Compound 8:30,546; found as: 30,486

Synthesis of 2DG-D-Cy5 (Compound 10): Compound 9 (50 mg, 1.0 eq, 0.003 mmol) was dissolved in DI water (1 mL) in a reaction vial and stirred at RT. Cy5 Azide (7.7 mg, 2.5 eq., 0.0075 mmol) dissolved in 1 ml of DMF was added to the stirred solution. The click reaction was initiated by adding a solution of CuSO₄.5H₂O (10 mol % per acetylene, dissolved in 0.1 mL DI water), followed by the addition of sodium ascorbate (15 mol % per acetylene, dissolved in 0.1 mL DI water) over 2 minutes. The reaction mixture was then stirred at 40° C. for 15 h. The progress of the reaction was monitored using HPLC. Upon completion, purification was performed by dialysis using a 1 kDa dialysis membrane against DI water for 15 hours. The purified product was lyophilized to obtain compound 10 in 90% yield.

¹H NMR (500 MHZ, DMSO-d₆) δ 8.47 (s, 8H), 8.36 (t, J=13.0 Hz, 5H), 8.19-7.75 (m, 56H), 7.65 (s, 7H), 7.39-7.27 (m, 6H), 7.18 (s, 16H), 7.02 (s, 2H), 6.65-6.52 (m, 7H), 6.30 (d, J=13.3 Hz, 7H), 5.31-5.07 (m, 16H), 5.04-4.63 (m, 70H), 4.61-4.20 (m, 154H), 4.20-3.95 (m, 86H), 3.95-3.41 (m, 1042H), 3.29-2.94 (m, 71H), 2.80 (s, 6H), 2.62 (s, 17H), 2.42-2.32 (m, 19H), 2.27-2.00 (m, 39H), 1.92-1.77 (m, 39H), 1.68 (s, 43H), 1.62-1.11 (m, 71H).

Synthesis of 2DG-D-Rosi-Cy5 (Compound 11): A solution of 2DG-D-Cy5 10 (30 mg, 1.0 eq, 0.0011 mmol) dissolved in DMF (0.2 mL) was added to a stirred solution of compound 4 (4.7 mg, 10 eq, 0.030 mmol) in DMF (0.1 mL) in a reaction vial. This was followed by sequential addition of CuBr (10 mol % per acetylene, 12 mg), PMDETA (4.5 mg), and THPTA (1.5 mg, cat.). The reaction mixture was then stirred at RT for 10 h. Upon completion, the mixture was purified by dialysis against DMF and DI water for 12 hours using a 1 kDa dialysis membrane. The aqueous solution was lyophilized to afford compound 11 in 95% yield (blue cotton-like solid).

¹H NMR (500 MHZ, DMSO-d₆) δ 8.47 (s, 8H), 8.36 (t, J=13.0 Hz, 10H), 8.10-8.00 (m, 37H), 7.98-7.75 (m, 55H), 7.64 (t, J=6.8 Hz, 10H), 7.47 (s, 14H), 7.32 (t, J=6.4 Hz, 11H), 7.18 (s, 16H), 7.09 (s, 15H), 6.80 (s, 14H), 6.66-6.46 (m, 28H), 6.30 (d, J=13.5 Hz, 8H), 5.30-4.69 (m, 83H), 4.62-4.37 (m, 143H), 4.35-4.19 (m, 44H), 4.17-3.95 (m, 111H), 3.93-3.70 (m, 156H), 3.71-3.33 (m, 669H), 3.49 (s, PEG), 3.19-2.85 (m, 155H), 2.71-2.51 (m, 80H), 2.33 (dd, J=16.6, 8.7 Hz, 62H), 2.26-2.07 (m, 62H), 2.03 (t, J=7.3 Hz, 33H), 1.91-1.64 (m, 146H), 1.63-1.40 (m, 87H), 1.38-1.15 (m, 78H), 1.07 (d, J=6.6 Hz, 55H).

¹³C NMR (125 MHZ, DMSO-d₆) δ 173.5, 173.1, 172.4, 166.0, 158.5, 154.7, 152.2, 148.1, 144.2, 140.3, 137.8, 131.9, 129.7, 124.7, 122.7, 114.2, 112.0, 110.6, 106.7, 106.3, 97.2, 97.1, 73.6, 72.1, 70.4, 70.3, 70.3, 70.2, 70.1, 70.1, 70.0, 69.4, 69.2, 69.2, 68.8, 68.4, 68.2, 66.3, 66.1, 64.0, 61.5, 49.8, 49.4, 49.0, 48.9, 38.8, 38.3, 36.2, 35.6, 35.3, 33.5, 28.9, 27.6, 27.4, 26.1, 25.6, 25.3, 25.1, 24.8, 12.6.

### In Vitro Drug Release and Stability Studies

### In Vitro Drug Release Studies

The studies to evaluate the release of Rosi from the 2DG-D-Rosi dendrimer were carried out under plasma conditions (pH 7.4, PBS and 2% FBS in PBS) and intracellular conditions (pH 5.5, citrate buffer with esterase). Accurately 2 mg of 2DG-D-Rosi was dissolved in 1 mL of above buffer solutions separately and incubated at 37° C. while shaking the solution mixtures continuously to simulate the natural physiological state. The sample solution of 100 μL was taken out at regular intervals and transferred to Eppendorf® tubes, quenched immediately with 100 μL of methanol, and stored at −20° C. until analyzed by HPLC. Rosi release was measured with respect to the calibration curve of free Rosi via HPLC.

### 2DG-D-Rosi Formulation Stability Studies

The formulation of 2DG-D-Rosi was developed in PBS at the concentration 50 mg/mL which was filtered through 0.4 μm sterile filters. The formulations were stored at 25° C. (RT) and 4° C. The stability and shelf life of 2DG-D-Rosi were analyzed using HPLC at predetermined time points (0 h, 1 Day, 7 Day, 14 Day and 28 Day).

### In Vitro Studies

### Hemocompatibility and Cytocompatibility of 2DG-D-Rosi

For the intended in vitro and in vivo use of the developed 2DG-D-Rosi dendrimer, ex-vivo hemocompatibility and in vitro cellular compatibility studies were carried out. For evaluating the effect of 2DG-D-Rosi on the RBCs, hemolysis assay was carried out. In brief, a 1:3 (RBC: 1× PBS) solution was taken in the micro-centrifuge tubes (MCTs) and kept at 37° C. for 1 h. Following this, the same volumes of 2DG-D (1000 μg/mL), 2DG-D-Rosi or Rosi (100, 500, and 1000 μg/mL) were added to the respective MCTs and incubated for ˜2 h at 37° C. in an incubator shaker at ˜100 rpm. After the incubation, the samples were centrifuged at 5000×g for 10 min, following this, 200 μL supernatant was taken and absorbance was recorded at 540 nm using a microplate reader (Synergy H1 hybrid multi-mode microplate reader, BioTek Instruments). RBCs treated with PBS and Triton®-x-100 were kept as a negative control and positive control, respectively. The percentage hemolysis was calculated using equation 1. All the measurements were done in triplicates, and hemolytic index was determined from the recommendation by ASTM E2524-08 standard.

$$Hemolysis ( \% ) = Absorbance of test sample / Absorbance of positive control \times 100 \qquad Eq . 1$$

Cytocompatibility studies were carried out using MTT assay as described in our previous work. For this, CATH.a, EOC 20, and HUVEC cells seeded in a 96 well-plate at density of 1×10⁴ cells/well were incubated with different concentrations of 2DG-D (1000 μg/mL), 2DG-D-Rosi or Rosi (100, 500, and 1000 μg/mL) for 24 h in a CO₂ incubator. After this, all the treated cells were incubated with MTT for 3 h till the purple-colored formazan crystals appeared. For dissolving these crystals, cell culture grade DMSO was added to each of the wells and incubated for 15 min. Finally, the absorbance was recorded at 570 nm using Synergy H1 hybrid multi-mode microplate. The percentage cytocompatibility was calculated using equation 2. All the measurements were done in triplicates with proper controls.

$$Cell viability ( \% ) = Absorbance of sample / Absorbance of control \times 100 \qquad Eq . 2$$

### In Vitro Neuronal Uptake of 2DG-D-Rosi

Prior to utilizing 2DG-D-Rosi dendrimers in an in vivo TBI mouse model, we investigated their cellular uptake and internalization under in vitro conditions to elucidate the underlying mechanism of uptake in CATH.a neuronal cell. These studies were performed with minor modifications following our previously published protocols. Briefly, CATH.a cells were cultured and seeded at a density of 2×10⁵ cells/well onto poly-L-lysine-coated glass cover slips. To assess the impact of specific cellular trafficking pathways, the cells were treated with various inhibitors: methyl β-cyclodextrin (MBCD, ˜2 mM), chlorpromazine (CPZ, 30 μM), and cytochalasin B (20 μM), and phloretin (˜1 mM). Treatments were conducted for 2 hours at 37° C. in a CO₂ incubator. Post-treatment, the inhibitor-containing medium was aspirated, and cells were washed with 1x PBS before being incubated with 2DG-D-Rosi-Cy5 for 6 hours under identical incubation conditions. Following incubation, cells were washed with cold PBS and fixed with 4% paraformaldehyde (PFA), prepared by mixing equal volumes of 8% PFA and 2x PHEMS buffer, at room temperature for ˜10 minutes. After aspiration of the fixative, cells were washed thoroughly with cold PBS and permeabilized using 0.1% Triton® X-100 for ˜10 minutes, followed by additional washes with PBS. For visualization, fixed and permeabilized cells were stained with DAPI (10 μL of 50 μM) for 15 minutes and subsequently stained with Pheno Vue® Fluor 568-Phalloidin (˜0.4 nM, 20 μL) for 1 hour. Each staining step was followed by washing with cold PBS. Imaging was performed using a Leica SP-5 confocal microscope.

### Quantitative TNF-α and IL-6 Assay

For determining the effect of 2DG-D-Rosi conjugates on the neuro-inflammation under in vitro conditions, the expression levels of tumor necrosis factor-alpha (TNF-α) and interleukin-6 (IL-6) were analyzed using an enzyme-linked immunosorbent assay (ELISA). The protocol was adapted from previously published literature with slight modifications. Briefly, CATH.a neurons (5×10⁴ cells/well) were seeded into 96-well plates and cultured overnight. The cells were pre-treated with lipopolysaccharide (LPS; 1 μg/mL) and hydrogen peroxide (H₂O2, 100 μM) and then incubated with 2DG-D-Rosi conjugates at varying concentrations (50, 100, and 250 μg/mL) for 24 hours at 37° C. in a CO₂ incubator. Following the treatment, the consumed culture media was collected and centrifuged at 12,000×g for 10 minutes to remove cellular debris. A 50 μL aliquot of the supernatant was added to ELISA plates pre-coated with primary antibodies for TNF-α and IL-6, respectively, and incubated for approximately 2 h. The cytokine concentrations (pg/mL) were determined using standard calibration curves provided in the ELISA kit, adhering to the manufacturer's instructions. All experiments were performed in triplicates, with appropriate controls.

### Greiss Reagent Assay for Nitric Oxide Estimation

The effect of dendrimer conjugates on reactive nitric oxide (NO) species was estimated using Greiss reagent-based assay, following published method with slight modification. For this, 5×104 CATH.a cells/well were seeded in a 96-well plate and grown in serum free media overnight. The media was changed, and the cells were treated with 2DG-D-Rosi dendrimer conjugates for 12 h at 37° C. in a CO₂ incubator. Following this, equal volumes (50 μL) of Greiss reagent (0.1%) and cell supernatant were mixed and incubated for 15 min for color change. Finally, absorbance was measured at 540 nm using a Thermo Scientific Multiskan Sky High® Microplate Reader. The final NO concentrations were determined following the nitrate standard calibration curve. All the measurements were taken in triplicates with proper controls.

### Annexin V/Propidium Iodide Assay

Apoptosis/necrosis was assessed in CATH.a cells using the FITC ApoScreen® Annexin V Apoptosis Kit. Cells were pre-treated with LPS (1 μg/mL) and H₂O₂ (100 μM) for 6 hours to mimic TBI-like conditions, followed by 2DG-D-Rosi treatment for 24 hours at 37° C., 5% CO₂. After incubation, cells were harvested, washed twice with cold PBS, and resuspended in 100 μL of 1× Annexin Binding Buffer. Staining was performed with 10 μL Annexin V-FITC and 10 μL Propidium Iodide (PI) for 15 minutes at 2-8° C. in the dark, followed by dilution with 380 μL 1× Annexin Binding Buffer. Samples were analyzed on an Attune NxT® Flow Cytometer using BL-1 (530/30 nm) for Annexin V-FITC and YL-1 (585/16 nm) for PI. Controls included unstained, Annexin V-only, and PI-only samples. Density plots were generated to classify viable, early apoptotic, necrotic, and late apoptotic/necrotic cells. The experiment was performed in triplicate (n=3), and data was analyzed using GraphPad Prism® with statistical significance determined by a two-tailed t-test.

### Mitochondrial Membrane Potential (MMP) Assay

The JC-1 Mitochondrial Membrane Potential (MMP) Assay was used to assess 2DG-D-Rosi effects on CATH.a cells under TBI-like conditions via flow cytometry and confocal microscopy. Cells were pre-treated with LPS (1 μg/mL) and H₂O₂ (100 μM), followed by 2DG-D-Rosi treatment for 24 hours at 37° C., 5% CO₂. After incubation, cells were stained with 2 μM JC-1 dye for 20 minutes at 37° C. in the dark. For flow cytometry, cells were analyzed on an Attune NxT® Flow Cytometer, with JC-1 monomers (green, depolarized mitochondria) detected in BL-1 (530/30 nm) and J-aggregates (red, polarized mitochondria) in YL-1 (585/16 nm). For confocal microscopy, cells were seeded on poly-L-lysine-coated coverslips, stained with JC-1, counterstained with DAPI, and imaged using a Leica SP-5 Confocal Microscope (20× water immersion magnification). The experiment was conducted in triplicate (n=3), and data were analyzed using GraphPad Prism® with statistical significance determined by a two-tailed t-test.

### Caspase Activity Assay

The Caspase-3/7 Activity Assay was conducted to assess 2DG-D-Rosi effects on apoptosis in CATH.a cells under TBI-like conditions via flow cytometry and confocal microscopy. Cells were pre-treated with LPS (1 μg/mL) and H₂O₂ (100 μM), followed by 2DG-D-Rosi treatment for 24 hours (flow cytometry) or 48 hours (confocal microscopy) at 37° C., 5% CO₂. For flow cytometry, 1×10^{5lls per sample were incubated with Pheno Vue®} 505 caspase-3/7 stain in fresh medium for 30 minutes, washed with PBS, and analyzed on an Attune NxT® Flow Cytometer using BL-1 (530/30 nm) for caspase activation detection. For confocal microscopy, 2×10⁵ cells per well were seeded on poly-L-lysine-coated coverslips, stained with caspase-3/7 reagent, fixed in 4% PFA, permeabilized with 0.1% Triton® X-100, and counterstained with DAPI. Coverslips were mounted with Vectashield Plus® antifade medium and imaged using a Leica SP-5 Confocal Microscope (20× water immersion lens). The experiment was conducted in triplicate (n=3), and statistical significance was determined using GraphPad Prism® with a two-tailed t-test.

### PPARγ Expression Analysis via Flow Cytometry and Immunofluorescence

Peroxisome proliferator-activated receptor gamma (PPARγ) expression in CATH.a cells were assessed using flow cytometry and immunofluorescence following 2DG-D-Rosi treatment. Cells were pre-treated with LPS (1 μg/mL) and H₂O₂ (100 μM), then incubated with 2DG-D-Rosi for 24 hours (flow cytometry) or 48 hours (immunofluorescence) at 37° C., 5% CO₂. For flow cytometry, 1×10⁵ cells per sample were fixed in 4% PFA, permeabilized with 0.2% Triton® X-100, blocked with 3% BSA, and incubated overnight at 4° C. with an anti-PPARγ antibody. After washing, cells were incubated with Pheno Vue® Fluor-488 IgG1 secondary antibody for 2 hours at 37° C., washed again, and analyzed on an Attune NxT® Flow Cytometer using BL-1 (530/30 nm) for PPARγ detection.

For immunofluorescence, 5×10⁴ cells per well were grown on poly-L-lysine-coated coverslips, treated, washed, and blocked with 3% BSA, followed by overnight incubation with an anti-PPARγ antibody at 4° C. on a 3D shaker. After washing, cells were incubated with Pheno Vue® Fluor-488 IgG1 secondary antibody for 2 hours at 37° C., washed, fixed, and permeabilized in 4% PFA and 0.2% Triton® X-100 for 10 minutes each, counterstained with DAPI, and mounted with VectaShield® antifade medium. Imaging was performed using a Zeiss SP5 Confocal Microscope (20× water immersion lens). The experiment was conducted in triplicate (n=3), and statistical significance was determined using GraphPad Prism® with a two-tailed t-test.

### In Vivo Studies

### Animals

Male and female C57BL/6 mice (2-3 month of age; Jackson Laboratory, Bar Harbor, ME) were in-house bred as previously described. All animals were housed under ambient conditions (20-22° C., 40-60% relative humidity, and a 12-hour light/dark cycle) with free access to food and water. All experiments followed the Guide for the Care and Use of Laboratory Animals, eighth edition, published by the National Research Council (National Academies Press, 2011). Experimental procedures were approved by the Institutional Animal Care and Use Committee (IACUC) of the University of Michigan (Protocol ID: PRO00010860).

### Impact Acceleration Model of TBI

On postnatal day 20-21 (P20-21), male (M) and female (F) animals (n=84, 42M/42F) from the same litter were randomized into Sham (n=20, 10M/10F) and TBI (n=64, 32M/32F) groups using a random number generator. Randomization was stratified by sex. The TBI animals underwent injury procedure as previously described. In brief, after fully anesthetized, the animal was placed chest-down on a platform with a trapdoor that supported the body weight of a mouse (˜7-10 g body weight) with little to no resistance or restraint upon impact. The animal's head was directly in the path of a falling weight. A weight (30g) was held at 1.0 meter above the platform and secured by a pin. The lab personnel pulled the pin, allowing the weight to fall vertically through a guide tube to strike the animal on the head in the midline between bregma and lambda (at approximately bregma-2.5 mm). Sham animals were anesthetized with 4% isoflurane without TBI impact. All animals were closely monitored postoperatively with weight and health surveillance recordings, as per IACUC guidelines.

2DG-D-Rosi-Cy5 Co-localization with Neurons

Male and female TBI mice (n=2 per sex) received intraperitoneal administration of fluorescent 2DG-D-Rosi-Cy5 (50 mg/kg, 100 μL) at 6-hour post-injury, and euthanized at 24-hour post-injection. Brains were removed, postfixed in 10% formalin for 48 hours, and then cryoprotected in 30% sucrose (in PBS). Coronal sections (20 μm, 1:6 series) were prepared on a cryostat (Leica Microsystems, IL, USA). Brain sections were incubated overnight at 4° C. with rabbit anti-NeuN (a neuronal marker; 1:250; Cat #ab177487; Abcam, MA. U.S.A.). Sections were subsequently washed and incubated with fluorescent secondary antibody (1:250; Life Technologies, MA, U.S.A.) for 2 hours at room temperature. The slides were dried, and cover slipped with fluorescent mounting medium with DAPI (Sigma-Aldrich, MO, USA). Images were acquired using Nikon Eclipse TS2R fluorescent microscope (Nikon, NY, USA).

In Vivo Rosiglitazone/dendrimer-rosiglitazone Administration

Mice in the TBI group were randomized into TBI+vehicle (n=20, 10M/10F), TBI+rosiglitazone (TBI+Rosi) (n=20, 10M/10F), and TBI+Dendrimer-rosiglitazone (TBI+2DG-D-Rosi) (n=20, 10M/10F) groups. Animals received intraperitoneal administration of free rosiglitazone (2 mg/kg, 100 μL), dendrimer-rosiglitazone (containing 2 mg/kg rosiglitazone, 100 μL) or vehicle (100 μL) at 6-hour post-injury. The mice from the sham group (n=20, 10M/10F) did not receive any intervention.

### Body Weight

Body weight was measured before injury (baseline) and at 1-day (d) post-treatment. The changes in the body weight were calculated as (Body weight)_{change}=(body weight) 1d-(body weight)_{baseline}.

### Behavioral Tests

All of the behavioral testing was performed between 7AM to 6 PM. Mice (n=20 per group, 10M/10F) were habituated in the test room for at least 30 min before the behavioral tests. The lab personnel were blinded to experimental groups.

Grip strength: Muscular strength was evaluated with a grip strength test using a grip strength meter (BIOSEB, FL, USA) before injury and at 1-d post-treatment. In brief, the grip strength meter was positioned horizontally and the animals were held by the tail and lowered towards the apparatus. The animals were allowed to grab the metal grid and were then pulled backwards in the horizontal plane. The force applied to the grid was recorded as the peak tension. Each animal underwent a grip strength test in three consecutive trials. The results were recorded and averaged for each animal. The change in the grip strength before and after injury was calculated as: (grip strength)_{change}=(grip strength)₂₄ₕ-(grip strength)_{baseline}

Rotarod: Sensorimotor coordination, endurance, and fatigue resistance was evaluated with a touchscreen five station accelerating Panlab RotaRod for mouse (BIOSEB, FL, USA) before injury and at 1-d post-treatment based on a published protocol. Each animal was situated on a stationary rod for 10 s, and the rod was then set in motion with an accelerating speed of 3-30 rpm. Each animal underwent three consecutive trials (5 min each). The latency to the first fall in each trial was recorded and averaged for each animal. The change in the latency to the first fall before and after injury was calculated as:

(Latency)_{change}=(latency)₂₄ₕ(latency)_{baseline}

Tail suspension test: The tail suspension test was performed at 1-d post treatment as previously described to evaluate depression-like behaviors. In brief, mice were suspended by taping their tails (three quarters of the distance from the base of the tail) to a vertical bar on a tail suspension stand. The animal tail was aligned with the bottom of the bar. The animals' activities were monitored continuously for 6 min. The time spent immobile over the 6 min period was quantified and compared among groups.

Light/dark box test: The light/dark box was purchased from Stoelting Co. (Wood Dale, IL, USA), and the test was performed as previously described. The test was performed at 1-d post treatment. In brief, mice were placed in the middle of the brightly illuminated chamber and were allowed to move freely between the light and dark chambers for 10 min. Video recording was used to record animal behaviors. The time spent in the light chamber was recorded and analyzed. Novel object recognition: The novel object recognition test was modified from published protocols and performed at 1-d post treatment. In brief, the test was composed of two trials. The mice explored two identical objects for 5 min during the “training trial” and then were placed back in their cages. After an inter-trial break of 4-hour, one of the previously exposed “old” objects was replaced with a new “novel” object, and the animals were allowed to explore these two objects for 5 min during the “probe trial”. The discrimination index for the probed trial was used to analyze the cognitive outcomes. Discrimination index=time spent exploring the novel object/(time spent exploring the old object+time spent exploring the novel object)×100%.

### Isolation of Primary Neurons

Animals were euthanized after completion of behavioral tests at 1-d post-treatment (n=12 per group, 6M/6F). Primary neuron isolation was performed as previously described. In brief, brains were harvested and rinsed in HBSS solution on ice. Meninges were removed and the area of injury (approximately between bregma+2 mm and bregma-1 mm) in the TBI mice, and the matching area in the sham mice were micro-dissected as previously described. Brain tissues were transferred to HABG solutions [60 mL HA, 1.2 mL B27, 0.176 mL Gln (0.5 mM final)], and minced (˜0.5 mm) on ice. Brain tissues were incubated in HABG solution for 8 min at 30° C. in a Boekel shaking incubator with a shaking speed of 90 rpm (Cole-Parmer, Vernon Hills, IL, USA). Tissues were transferred to papain solutions [12 mg papain solids per 6 mL HA-Ca, 0.015 mL Gln (0.5 mM final)], and incubated for 30 min at 30° C. in a shaking incubator with a shaking speed of 90 rpm. Tissues were washed in HABG solution for 5 min at room temperature, triturated with sterile pipette for 45 s, and kept at room temperature for 1 min. The supernatants were collected, and the trituration was repeated two times. The supernatants were combined and centrifuged in OptiPrep™ Density Gradient Medium at 800 xg for 15 min at 22° C., and the fractions of neurons were collected as previously described. Cells were washed in HABG solutions and centrifuged at 200×g for 2 min at 22° C. Supernatants were removed, and cells were washed in HBSS solution and centrifuged at 200 xg for 2 min at 22° C. Cell pellets were harvested for RNA isolation.

RNA Isolation and Quantitative Real-time Polymerase Chain Reaction (qPCR)

The mRNA expression of TNF-α (tumor necrosis factor alpha), IL-1β (interleukin-1 beta), TLR4 (Toll-like receptor 4), NLRP3 (NLR Family Pyrin Domain Containing 3), IL-13, and Fas were measured. Total RNA was extracted using TRIZOL (Sigma-Aldrich, MO, USA), according to manufacturer's instructions. RNA samples were quantified using the Nanodrop® ND-2000 Spectrophotometer (Thermo Fisher Scientific, MA, USA). Single-stranded complementary DNA (cDNA) was reverse transcribed from RNA using the High-Capacity cDNA Reverse Transcription Kit with RNase inhibitor (Thermo Fisher Scientific, MA, USA). qPCR was performed with iTaq™ Universal SYBR® Green Supermix (Bio-Rad, CA, USA) with CFX connect real-time PCR detection system (Bio-Rad, CA, USA). Amplification conditions included 30 sec at 95° C., 40 cycles at 95° C. for 5 sec, and 60° C. for 30 sec. Primers were custom designed and ordered from Integrated DNA Technology (Coralville, IA, USA). The comparative threshold cycle (Ct) method was used to assess differential gene expressions.

The sham group was the reference group, and glyceraldehyde 3-phosphate dehydrogenase (Gapdh) was the housekeeping gene. Gene expression levels for each sample were normalized to the expression level of Gapdh within a given sample (ΔCt); the differences between sham and TBI groups were used to determine the ΔΔCt. The 2-ΔΔCt gave the relative fold changes in gene expression.

### Immunohistochemistry

Animals were euthanized after completion of behavioral tests at 1-d post-treatment (n=8 per group, 4M/4F), and transcardially perfused with PBS. Brains were removed, postfixed in 10% formalin for 48 h, and then cryoprotected in 30% sucrose (in PBS). Coronal sections (20 μm, 1:6 series) were prepared on a cryostat (Leica Microsystems, IL, USA). Brain sections were washed 3 times in PBS for 5 min each, incubated in 0.001% Fluoro-Jade® C (FJ-C) (Cat #TR-160-FJC, Biosensis, CA, USA) for 10 min, washed 6 times in PBS for 15 min each, and incubated with rabbit anti-NeuN (1:250; Cat #ab177487; Abcam, MA, U.S.A.). Sections were subsequently washed and incubated with fluorescent secondary antibodies (1:250; Life Technologies, MA, U.S.A.) for 2 hours at room temperature. The slides were dried, and cover-slipped with fluorescent mounting medium with DAPI (Sigma-Aldrich, MO, USA).

### Histology Quantification

Images (40X, 5 images/animal) were randomly acquired from the area of injury using the Nikon Eclipse® TS2R fluorescent microscope (Nikon, NY, USA), and the camera settings were kept the same for all the experimental groups. All slides and images were coded, and the analysis was performed with personnel blinded to the experiments. The co-localization of FJC+NeuN⁺ cells were analyzed using “Coloc 2” function in Fiji ImageJ® (National Institutes of Health, NIH) as previously described. In brief, images were split into separate channels and were converted to 8-bit. Background subtraction was performed using Fiji ImageJ® following the manufacturer's instructions. The co-localization was analyzed using Kendall's Tau Rank Correlation test, and Costes' randomization was set at 10. To ensure accuracy of analysis, data were manually checked with co-localization analysis as described previously.

### Statistical Analysis

Data was analyzed using GraphPad Prism® 6 (Version 6.04; CA, USA). All data were presented as mean±SEM. Kolmogorov-Smirnov normality test was used for normality measurement. One-way and two-way ANOVA and Bonferroni post hoc test were used for multiple group comparisons. Statistical significance was set at p<0.05 for all analyses.

### Results and Discussion

### Synthesis and Physicochemical Characterization of 2DG-D-Rosi Conjugate

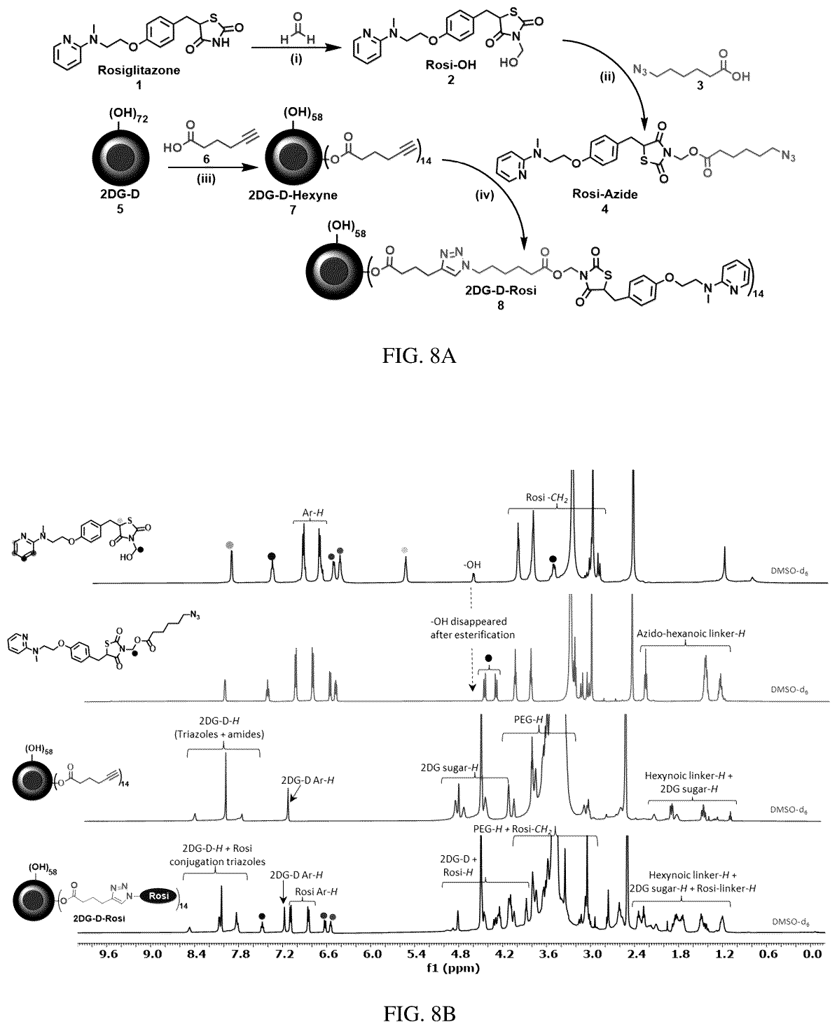

The synthesis of the 2DG-D-Rosi was initiated by the modification of Rosi with an azide terminating linker for subsequent conjugation on alkyne-terminating 2DG-D, utilizing copper-catalyzed azide-alkyne click (CuAAC) chemistry. More specifically, Rosi was first reacted with formaldehyde to generate Rosi-OH (2), as illustrated in FIG. 8A. The ¹H NMR spectra of Rosi-OH displayed signals between 8 3.67-3.57 ppm, confirming the presence of the —CH₂OH group attached to the nitrogen atom (FIG. 8B). Subsequently, Rosi-OH (2) was converted into Rosi-Azide (4) by the attachment of clickable linker 6-azido hexanoic acid (3) using EDC-DMAP as the coupling agent. The ¹H NMR spectrum of Rosi-Azide (4) revealed characteristic signals of the aliphatic chain from azido-hexanoic acid, with proton peaks observed between 8 2.32-1.28 ppm. Additionally, the two protons (—CH₂) attached to the nitrogen of the thiazolidinedione ring appeared as distinct doublets at 8 4.52 and 8 4.37 ppm (FIG. 8B).

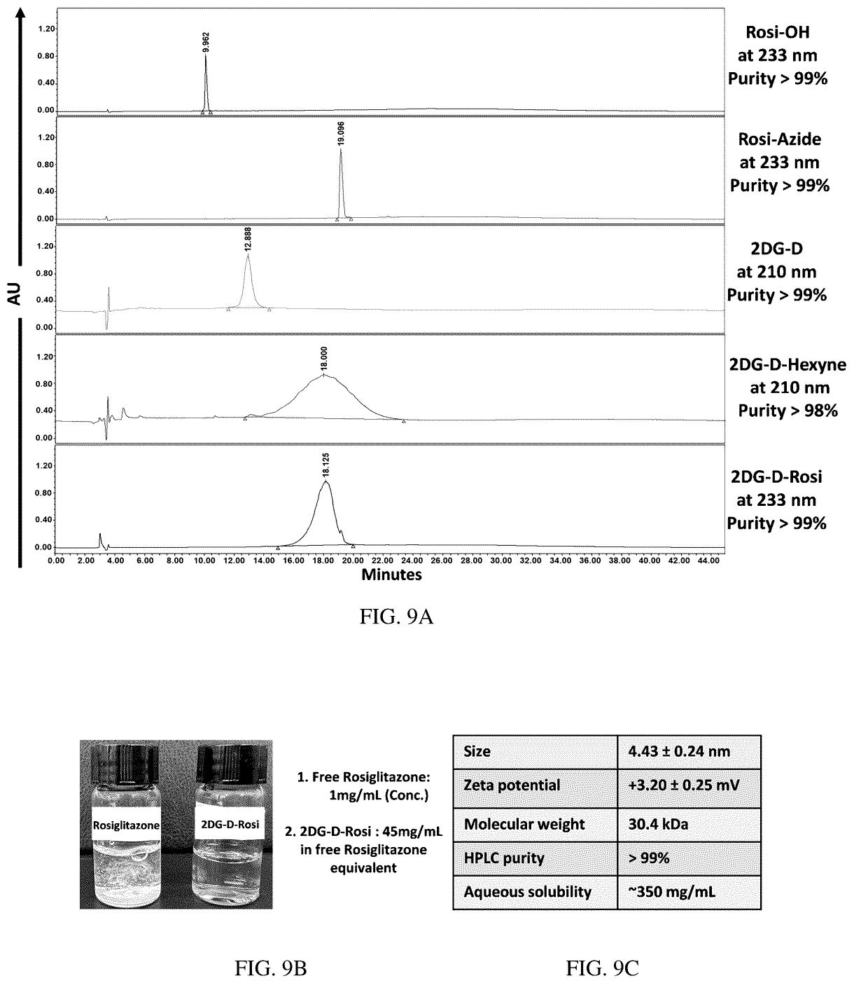

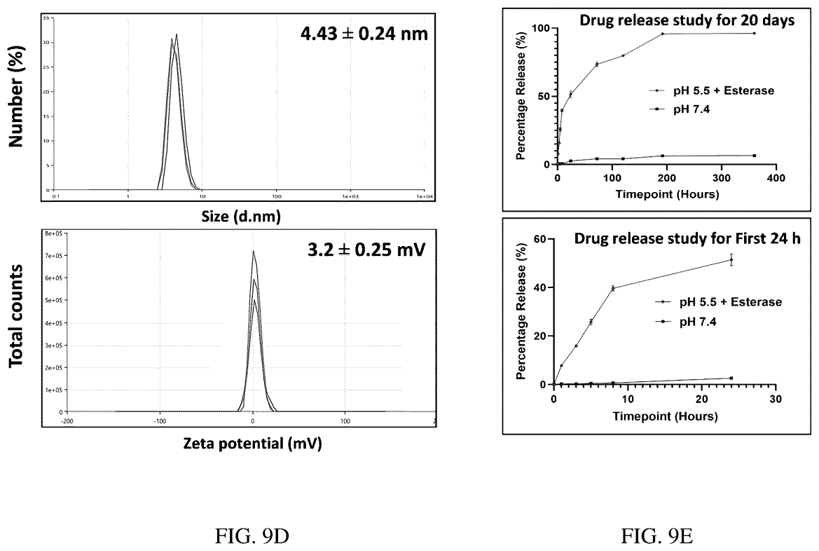

Next, we synthesized 2DG-D-Hexyne (7), functionalizing 2DG-D with hexyne groups through esterification (FIG. 8). Hexynoic acid (6) was reacted with the hydroxyl groups on the surface of the dendrimer using the EDC and DMAP as coupling reagents, resulting in the formation of 2DG-D-Hexyne (7). The number of alkyne groups on the dendrimer can be tailored to achieve the desired drug loading capacity. The formation of 2DG-D-Hexyne was confirmed using HPLC, which demonstrated a shift in retention time compared to the unfunctionalized dendrimer, indicating the formation of product (FIG. 9A). Additionally, ¹H NMR spectra revealed characteristic aliphatic proton signals in the range of 8 2.32 to 1.28 ppm, confirming the presence of the methylene protons of hexynoic acid linker (FIG. 8B). The alkyne groups on the dendrimer were then conjugated with Rosi-Azide (4) via CuAAC. This reaction, performed in an anhydrous environment using CuBr and PMDETA in dry DMF, yielded 2DG-D-Rosi (8) with greater efficiency compared to aqueous conditions using copper sulfate and sodium ascorbate, likely attributed to the enhanced stability of the Rosi-Azide in the absence of water. The formation of the triazole rings from the click reaction was confirmed by the appearance of new peaks in the ¹H NMR spectra, observed between 8 8.02 to 7.82 ppm, which are characteristic of triazole protons. Additionally, the aromatic proton signals from Rosi at 67 7.08 and 6.84 ppm further validated the successful attachment of approximately ˜14 drug molecules to the dendrimer (FIG. 8B). The purity of the resulting 2DG-D-Rosi was ˜99%, as determined by HPLC analysis, which also showed a significant shift in retention time from 18.0 min (2DG-D-Hexyne) to 18.12 min (2DG-D-Rosi), further confirming successful conjugation (FIG. 9A). The drug loading was assessed by NMR integration method, suggesting a drug loading of 12-13% by weight, corresponding to the attachment of ˜14 Rosi molecules per dendrimer. Detailed characterization of intermediates and final 2DG-D-Rosi conjugate are carried out via NMR and mass spectroscopy techniques and the purity was determined using HPLC.

Rosi exhibits significant challenges in terms of solubility and is sparingly soluble in water. This limited solubility restricts bioavailability and therapeutic applications. However, conjugation with the 2DG-D dendrimer markedly enhanced the solubility of Rosi (FIG. 9B). The physicochemical properties for 2DG-D-Rosi are detailed in FIG. 9C. The zeta potential of 2DG-D-Rosi was determined to be nearly neutral (3.2+0.25 mV,) and the size distribution was measured at 4.43+0.24 nm (FIG. 9D), characteristics that facilitates extravasation through leaky blood vessels at the brain injury site and efficient movement through the brain tissue parenchyma. Additionally, the small size of conjugate also supports renal clearance from off-target organs, thereby ensuring minimizing of systemic toxicity.

### In Vitro Drug Release and Shelf Stability Studies

Rosi was conjugated to 2DG-D through an enzyme cleavable ester linkage for intracellular drug release. The drug release behavior of 2DG-D-Rosi was evaluated under two biologically relevant conditions to provide insight into its potential performance in vivo. First, drug release was studied at physiological pH (7.4) in phosphate-buffered saline (PBS) at 37° C., which simulates plasma conditions. The conjugate demonstrated stability in plasma conditions with less than 5% release over 2 weeks, ensuring minimal premature drug leakage during transport to the target site. Second, the drug release was evaluated under acidic conditions (pH 5.5) in the presence of esterase to mimic the intracellular endosomal/lysosomal environment.

The acidic pH reflects the characteristic environment of endosomes/lysosomes, while esterase was included to simulate enzymatic activity that could trigger drug release upon cellular uptake. At intracellular conditions, ˜50% of the drug was released within 2-3 days, and ˜80% released over 12 days, demonstrating a sustained intracellular release profile of the conjugate (FIG. 9E).

Next, the shelf-life stability of 2DG-D-Rosi formulation in PBS was evaluated under two storage conditions: at 25° C. (room temperature, RT) and 4° C. HPLC data demonstrated exceptional stability, with the purity of 2DG-D-Rosi remaining unchanged over 28 days, varying negligibly between 99.44% and 98.09%, with no significant shifts in retention time confirming the structural integrity of the formulation under these conditions. The lack of notable changes in retention time strongly suggests that the conjugated rosiglitazone remained stably bound to the dendrimer, with no detectable release of the free drug during storage. The stability of 2DG-D-Rosi under these storage conditions has significant implications for its clinical applications. The ability to retain its purity and structural integrity for extended periods ensures reliable handling, transport, and storage, which are critical for transitioning from preclinical to clinical settings. Furthermore, the negligible degradation or release of the free drug minimizes concerns regarding toxicity or reduced efficacy due to drug release during storage.

To investigate the in vitro and in vivo neuronal uptake of 2DG-D-Rosi via confocal microscopy, we attached a near infra-red fluorescent tag cyanine 5 (Cy5) to develop 2DG-D-Rosi-Cy5 (11). The synthesis began by conjugating Cy5 to 2DG-D through the modification of its surface hydroxyl groups with 5-hexynoic acid using EDC-DMAP, resulting in 2DG-D-Hexyne (9). Subsequently, 2DG-D-Hexyne (9) was reacted with Cy5 azide via CuAAC to form 2DG-D-Cy5 (10). Characterization by ¹H NMR confirmed the incorporation of around 2-3 Cy5 molecules per dendrimer. Next, 2DG-D-Rosi-Cy5 (11) was synthesized by reacting 2DG-D-Cy5 (10) with rosi-azide (4) in the presence of anhydrous CuBr, again utilizing the CuAAC reaction. The reaction progress was monitored by HPLC, and once Rosi-Azide was fully consumed, the reaction was stopped, and the product was purified using size exclusion chromatography. The successful formation of 2DG-D-Rosi-Cy5 was confirmed by ¹H NMR, which exhibited proton signals from the pyridyl ring of Rosi at 8 6.29 ppm confirming the successful attachment of 7-8 Rosi molecules. A distinct shift in retention time was observed between 2DG-D-Cy5 (17.34 minutes) and the synthesized 2DG-D-Rosi-Cy5 (17.91 minutes), further validating the conjugation of Rosi.

### Role of GLUT Transporters in the Neuronal Uptake of 2DG-D-Rosi Conjugate

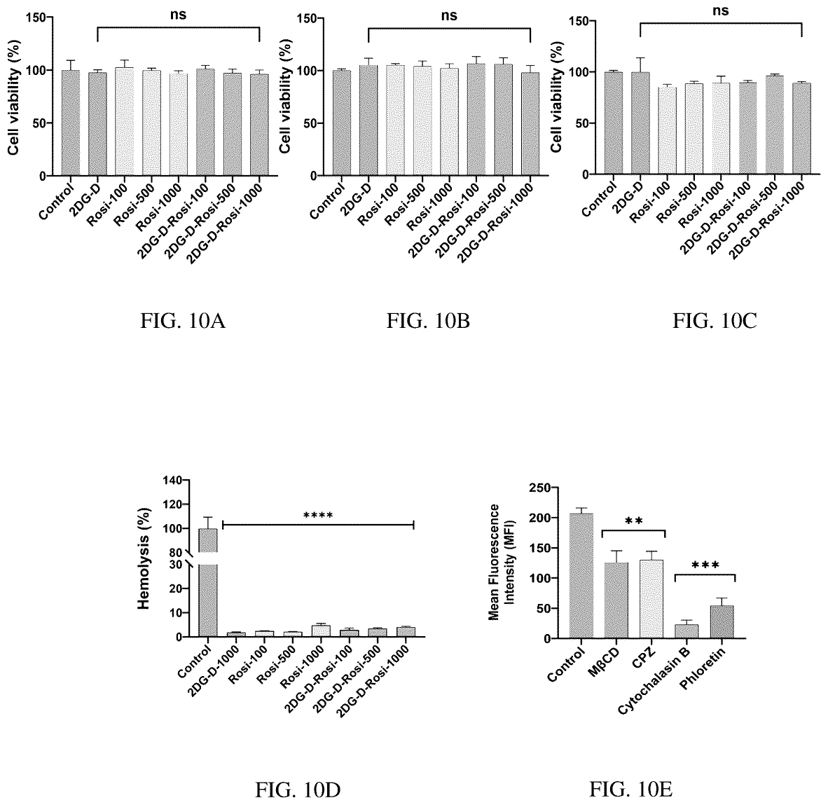

Before using 2DG-D-Rosi in either in vitro or in vivo experiments, we assessed the compatibility of the dendrimer conjugates with red blood cells (RBCs) and three representative cell lines viz., CATH.a neurons, human umbilical vein endothelial cells (HUVECs), and EOC 20 microglial cells. An MTT cell viability assay was conducted to evaluate the cytotoxicity of 2DG-D-Rosi on these cell lines. No toxic effects were observed at all tested concentration of 2DG-D, Rosi, or the 2DG-D-Rosi conjugate (FIG. 10A-C). Additionally, no significant hemolysis was observed in RBCs treated with the 2DG-D-Rosi conjugate at all tested concentrations, with an overall hemolysis percentage below 5%, suggesting excellent compatibility with RBCs (FIG. 10D) and confirming that 2DG-D-Rosi conjugates were safe to use further for carrying out in vitro/in vivo neuronal uptake and efficacy studies.

Previous reports have shown that glucose transporters (GLUTs), especially GLUT3 are key protein transporters that mediate the uptake of glucose and glucose-based nanocarriers. To investigate the internalization mechanism of the 2DG-D-Rosi-Cy5 conjugate, we conducted uptake studies in the presence of trafficking inhibitors. The results revealed no significant reduction in the Cy5 (red fluorescence) signal when cells were treated with methyl-β-cyclodextrin (MBCD) or chlorpromazine (CPZ), compared to the untreated control, indicating that caveolae- and clathrin-dependent endocytosis were not significantly involved in the uptake of the 2DG-D-Rosi-Cy5 dendrimer by neuronal cells (FIG. 10E). In contrast, treatment with cytochalasin B and phloretin, both GLUT inhibitors, led to a marked decrease in the uptake of 2DG-D-Rosi-Cy5, with cytochalasin B causing a more substantial reduction in the Cy5 signal compared to phloretin. This suggests a predominant role of GLUT transporters in the uptake of 2DG-D-Rosi-Cy5. Quantitative analysis using ImageJ® further confirmed these observations, with the mean fluorescence intensity (MFI) for MBCD-treated cells (0.9-fold, p<0.005) being similar to the untreated control (1-fold), while the MFI was reduced to 0.86-fold (p<0.006), 0.11-fold (p<0.0001), and 0.26-fold (p<0.0003) for CPZ, cytochalasin B, and phloretin inhibitor-treated cells, respectively. These findings collectively indicate that the neuronal uptake of 2DG-D-Rosi-Cy5 is primarily mediated through GLUT transporters, with minimal contributions from caveolae- or clathrin-dependent pathways (FIG. 10E).

### 2DG-D-Rosi Attenuates Inflammatory Response and Provides Neuroprotection in LPS: H2O2 Induced CATH.a Cells

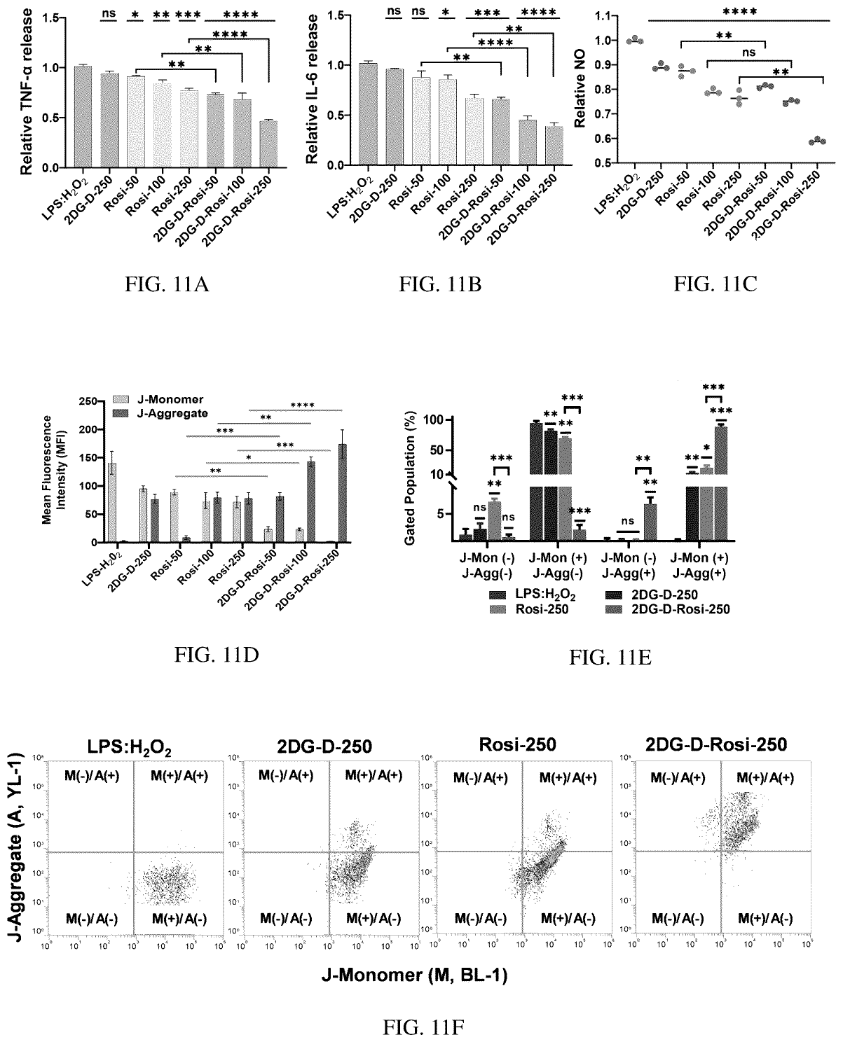

Shortly after TBI, there is a rapid surge in proinflammatory mediators in the brain, particularly TNF-α. Persistent elevation of TNF-α in injured brain tissue has been shown to exacerbate trauma by increasing oxidative stress, disrupting BBB, and amplifying inflammation, which further intensifies the severity of the traumatic injury. Furthermore, the elevated levels of IL-6 has also been observed to worsen the TBI condition by disruption of BBB and causing cerebral edema. Hence to explore the impact of 2DG-D-Rosi on the TNF-α and IL-6 levels in the CATH.a neuronal cells, we induced TBI-like conditions using LPS: H2O2 and treated the cells with 2DG-D-Rosi dendrimer conjugates. The results revealed a dose-dependent decrease in TNF-α and IL-6 levels in the treated cells compared to the untreated control group. Specifically, after treatment, TNF-α levels showed a slight reduction from 1-fold in the control (LPS: H₂O₂) group to 0.94-fold in empty 2DG-D at 250 μg/mL (2DG-D-250). At concentrations of 100, 250, and 500 μg/mL, TNF-α levels significantly decreased to 0.91, 0.85, and 0.77-fold for Rosi, and 0.73, 0.68, and 0.47-fold for 2DG-D-Rosi, respectively, though the decrease was much more pronounced in 2DG-D-Rosi group than in only Rosi treated group (FIG. 11A). Similarly, IL-6 levels followed a comparable trend, with the control group with ˜1-fold, while 2DG-D-250 reduced IL-6 to 0.96-fold. Rosi treatment led to reductions of 0.88, 0.86, and 0.67-fold, while 2DG-D-Rosi exhibited the most significant decreases, reducing IL-6 release to 0.66, 0.45, and 0.39-fold at 50, 100, and 250 μg/mL, respectively (FIG. 11B). These findings demonstrate a dose-dependent reduction in the inflammatory response in neurons following treatment with 2DG-D-Rosi dendrimer conjugates, highlighting the superior efficacy of 2DG-D-Rosi in mitigating neuroinflammation compared to Rosi.

Nitric oxide (NO), synthesized by nitric oxide synthase (NOS) in neurons, plays a critical role in neuroprotection under controlled physiological conditions. However, in TBI condition, heightened neuroinflammation leads to the excessive production of NO, which has deleterious effects, including neuronal damage and cell death. To address this, we investigated the impact of 2DG-D-Rosi dendrimer conjugates on NO generation in neurons. Our quantitative analysis revealed that treatment with 2DG-D-Rosi resulted in a dose-dependent reduction in NO production across all treated cells. Compared to the LPS: H₂O₂, NO generation was significantly decreased in all experimental groups. Notably, a comparison between free

Rosi and 2DG-D-Rosi demonstrated a more pronounced difference in reduction in NO levels for 2DG-D-Rosi. Specifically, the relative NO concentrations for Rosi and 2DG-D-Rosi were 0.89 and 0.82-fold (p<0.01), 0.78 and 0.76-fold (not significant), and 0.76 and 0.58-fold (p<0.01) at 50, 100, and 250 μg/mL, respectively (FIG. 11C). These findings highlight the superior efficacy of 2DG-D-Rosi in modulating NO levels compared to Rosi alone. The significant reduction in NO generation observed with 2DG-D-Rosi can likely be attributed to the enhanced intracellular delivery of 2DG-D-Rosi.

2DG-D-Rosi Protects Neurons from Mitochondrial Dysfunction by Maintaining Mitochondrial Membrane Potential (MMP)

Following TBI, there are several pathophysiological and biochemical changes occurring at trauma site, which result in the cascade of secondary injury occurring hours or days after the insult. These changes further result in the activation of cellular processes like increased reactive oxygen species (ROS) generation, post-traumatic neuroinflammation, mitochondrial dysfunction etc., which ultimately lead to cell death. To evaluate the impact of 2DG-D-Rosi on mitochondrial homeostasis, we performed JC-1 dye-based confocal microscopy and flow cytometry studies. Following the induction of TBI-like conditions and subsequent dendrimer treatment, JC-1 dye was used to assess changes in MMP in the presence of 2DG-D-Rosi dendrimer conjugates. Quantitative ImageJ® analysis of the confocal micrographs revealed that untreated control cells exhibited strong green fluorescence, indicative of J-monomer formation due to mitochondrial dysfunction and depolarized membranes (FIG. 11D). In contrast, treatment with Rosi and 2DG-D-Rosi at concentrations of 50, 100, and 250 μg/mL demonstrated a dose-dependent decrease in the mean fluorescence intensity (MFI) of J-monomers. In the control group the MFI was ˜141, which dropped to ˜89, 74 and 72 with Rosi treatment, and to 24, 23, and 2.3 with 2DG-D-Rosi treatment. Concurrently, a significant increase in red fluorescence intensity was observed, indicating enhanced mitochondrial polarization and improved membrane potential, suggesting of restored mitochondrial health. Notably, cells treated with 2DG-D-Rosi conjugates exhibited a substantial increase in red fluorescence (J-aggregates), where the MFI increased from ˜2.7 in the LPS: H₂O₂ group to ˜78.1 and ˜174 at 250 μg/mL for Rosi and 2DG-D-Rosi, respectively, highlighting superior restoration of MMP and mitochondrial function by 2DG-D-Rosi in comparison to the Rosi (FIG. 11D). Flow cytometry analysis further confirmed these findings, showing that LPS+H₂O₂ treatment resulted in approximately 95.13% depolarized mitochondria (J-Mon+/J-Agg-), with minimal polarized (˜0.23%) or healthy (˜0.24%) mitochondria. Treatment with 2DG-D-250 and Rosi-250 significantly reduced depolarized mitochondria (˜82.00% and ˜69.73%, respectively, p<0.01), while increasing healthy mitochondria (˜12.61%, p<0.01 and ˜21.54%, p<0.01). Notably, the treatment with 2DG-D-Rosi drastically reduced depolarized mitochondria (˜2.09%, p<0.001) while significantly increasing both polarized (˜6.89%, p<0.01) and healthy mitochondria (˜88.56%, p<0.001), demonstrating its superior mitochondrial protective effects in the neuronal cells compared to free drug (FIG. 11E, 11F).

Overall, these findings confirm that 2DG-D-Rosi dendrimer conjugates significantly improve mitochondrial health and exhibit enhanced efficiency in neuronal rescue by mitigating LPS: H₂O₂-induced mitochondrial damage.

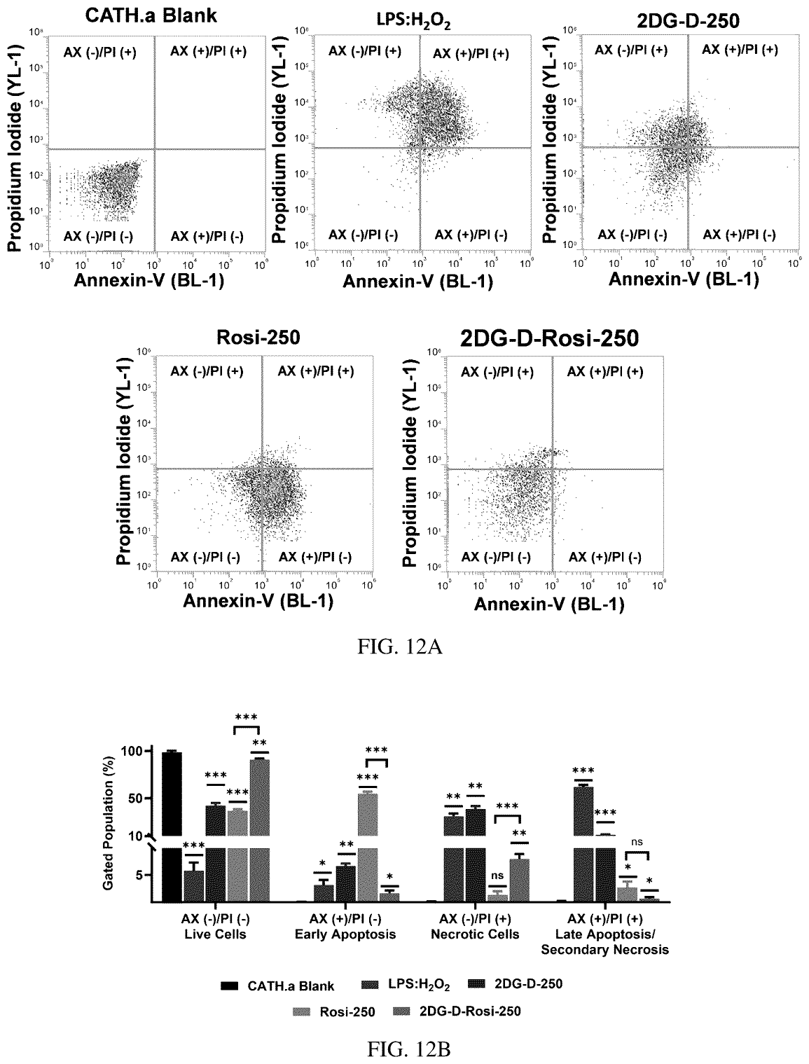

2DG-D-Rosi Attenuates Apoptosis and Necrosis in CATH.a Cells Under TBI-like Conditions Apoptosis plays a critical role in neuronal injury and repair, particularly under conditions of oxidative and inflammatory stress. To assess the cytotoxic effects of 2DG-D-Rosi in CATH.a cells, we performed an Annexin V-FITC/PI apoptosis assay using flow cytometry (FIG. 12A-B). Untreated cells exhibited high viability (˜98.7% AX-V-/PI-) with minimal apoptosis/necrosis (˜0.09% AX-V-/PI+), confirming a baseline low apoptotic rate. In contrast, LPS: H₂O₂ treatment significantly reduced cell viability (˜5.78%, p<0.001) and induced a sharp increase in necrosis (˜31.07%, p<0.001) and late apoptosis (˜62.1%, p<0.001), confirming severe oxidative stress-induced cell death. 2DG-D alone at 250 μg/mL displayed modest protective effects, increasing viability (˜42.2%, p<0.001) while reducing necrosis (˜38.56%) compared to LPS: H₂O₂, but still exhibited notable apoptotic activity (˜6.62% AX-V+/PI-). Rosi alone at 250 μg/mL resulted in a significant shift towards apoptosis (˜54.6%, p<0.001), but maintained a small necrotic population (˜1.67%, ns), similar to untreated CATH.a cells, indicating partial protection. Notably, 2DG-D-Rosi treatment resulted in the highest viability (˜90.63%, p<0.001) with significantly reduced apoptosis (˜1.63%) and necrosis (˜8.01%), suggesting its neuroprotective potential in mitigating TBI-induced oxidative damage. These results indicate that 2DG-D-Rosi effectively prevents apoptosis and necrosis, likely by stabilizing mitochondrial function and reducing oxidative stress. The significant differences observed across conditions suggest a mechanistic role in mitigating apoptotic and necrotic cell death pathways by 2DG-D-Rosi.

### 2DG-D-Rosi Conjugates Alleviates Caspase Activity and Upregulates PPARγ Expression in Neurons

In the later stages of TBI, apoptosis plays a pivotal role in the pathophysiology of the condition, and inhibiting this process can aid in neuronal recovery. During TBI, the activation of caspase-3, the key executioner caspase, contributes to apoptosis and exacerbates neuronal damage. Furthermore, caspase-3 promotes the proteolysis of DNA repair and cytoskeletal proteins, leading to DNA damage and further apoptosis. To evaluate the effect of 2DG-D-Rosi conjugates on alleviating caspase activity, confocal microscopy studies were conducted. Following the induction of TBI-like conditions, neurons were treated with varying concentrations of 2DG-D-Rosi dendrimer conjugates for 24 hours, after which confocal microscopy was performed. Quantitative analysis using ImageJ® of the confocal images revealed that, compared to the control group (100% caspase activity), caspase activity was reduced in the Rosi-treated groups (˜75%, ˜63%, and ˜68%) and in the 2DG-D-Rosi-treated groups (˜59%, ˜42%, and ˜29%) at concentrations of 50, 100, and 250 μg/mL, respectively. 2DG-D-Rosi treatments suppressed caspase activity significantly more than Rosi in a dose-dependent manner, highlighting its superior efficacy (data not shown).

The flow cytometry analysis further confirmed these findings, showing a dramatic increase in caspase activity following LPS: H₂O₂ exposure (MFI: ˜ 13,865), compared to baseline levels in untreated cells (MFI: ˜ 112). Treatment with 2DG-D and Rosi at 250 μg/mL significantly reduced caspase activation (MFI: ˜ 8,158 and MFI: ˜ 4,149, respectively, p<0.01). Notably, 2DG-D-Rosi exhibited the most significant suppression of caspase activity (MFI: ˜ 2,583, p<0.001), confirming its enhanced apoptotic inhibition. Compared to free Rosi, which lowered caspase activity by ˜70%, 2DG-D-Rosi treatment led to a further ˜38% reduction (p<0.001), demonstrating its superior caspase-inhibitory effects. Additionally, the percentage of caspase-positive cells was significantly lower in the 2DG-D-Rosi group compared to all other treatments, further reinforcing its potential to prevent apoptosis. These results emphasize that 2DG-D-Rosi more effectively inhibits caspase-3 activation than free rosiglitazone, suggesting a greater neuroprotective potential in mitigating TBI-induced apoptotic cell death.

### Neuronal Stimulation by 2DG-D-Rosi Upregulates PPARγ Expression

PPARγ plays a pivotal role in neuroprotection by regulating neuroinflammation and astrocyte polarization through anti-apoptotic and antioxidant mechanisms. In this study, we evaluated whether neuronal stimulation with 2DG-D-Rosi dendrimer conjugates could modulate PPARγ expression levels. Following treatment with 2DG-D-Rosi, a dose-dependent increase in PPARγ expression was observed, as evident from qualitative confocal micrographs. Quantitative analysis using ImageJ® software revealed that compared to the LPS: H₂O₂ treated group (1-fold), treatment with 2DG-D at 250 μg/mL resulted in a notable increase in PPARγ expression (1.8-fold, p<0.05). Furthermore, when comparing the relative levels of PPARγ expression between free Rosi and 2DG-D-Rosi at concentrations of 50, 100, and 250 μg/mL, the Rosi-treated group showed increases of 1.82, 1.79, and 1.9-fold, respectively. In contrast, 2DG-D-Rosi demonstrated significantly higher PPARγ expression levels of 2.1 (p<0.01), 2.2 (p<0.01), and 3.1-fold (p<0.01), at 50, 100, and 250 μg/mL (data not shown). Flow cytometry analysis further validated these findings, showing that LPS+H₂O₂ treatment resulted in a significant reduction in PPARγ expression (MFI: ˜ 206) compared to untreated neurons (MFI: ˜ 112). Treatment with 2DG-D and Rosi significantly increased PPARγ expression (MFI: ˜ 318 and MFI: ˜ 556, respectively, p<0.01). However, 2DG-D-Rosi treatment resulted in much significant upregulation of PPARγ expression (˜1111, p<0.01), demonstrating its superior ability to enhance PPARγ signaling. Compared to free Rosi, 2DG-D-Rosi led to nearly a 2-fold greater increase in PPARγ expression (p<0.01), reinforcing the advantage of dendrimer-based delivery. These results further highlight the enhanced neuroprotective effects of 2DG-D-Rosi, which may be linked to its ability to efficiently modulate inflammatory responses and protect against oxidative stress in neurons.

### 2DG-D-Rosi-Cy5 Co-localizes With Neurons at the Site of Injury From Systemic Administration in a Pediatric Mouse Model of TBI

We have previously demonstrated that systemically administered cy5 labeled 2DG-D accumulates in neurons at the site of brain injury. To evaluate if conjugation of Rosi on 2DG-D maintains this targeting capability, the cellular co-localization of Cy5 labeled 2DG-D-Rosi in the brain of juvenile TBI mice was assessed. We used an impact acceleration TBI model that reliably induces diffuse axonal injury in the absence of skull fractures and parenchymal focal lesions. This model replicates the pathophysiology that is commonly observed in humans caused by falls. Studies have shown that falls are the leading cause of TBI in children, thus this model is suitable for secondary insult modeling with the adaptability for mild/moderate injury through alteration of height and/or weight. Fluorescently labeled 2DG-D-Rosi-Cy5 conjugates were administered at 6-hour post-injury and animals were euthanized at 24-hour post-injection. We found that 2DG-D-Rosi-Cy5 was co-localized with neurons in the injured brain regions, whereas, 2DG-D-Rosi-Cy5 uptake in the non-injured brain regions of the TBI animals was barely detectable (data not shown).

### 2DG-D-Rosi Improved Behavioral Outcomes

To evaluate the efficacy of Rosi and 2DG-D-Rosi on body weight, we compared the change in the body weight before TBI (baseline) and at 1-d post-treatment. Upon two-way ANOVA analysis [sex (male, female), treatment (sham, TBI+vehicle, TBI+Rosi, TBI+2DG-D-Rosi)], there were significant differences in the (body weight)_{change} based on treatment [F_{(3,72)}=35.85, p<0.0001]. In males, the body weight significantly decreased in TBI+vehicle (p<0.0001), TBI+Rosi (p<0.0001) and TBI+2DG-D-Rosi (p<0.01) groups, compared with the sham group. Moreover, the body weight significantly decreased in the TBI+vehicle group, compared with the TBI+2DG-D-Rosi group (p<0.05). In females, the body weight significantly decreased in TBI+vehicle (p<0.0001) and TBI+Rosi (p<0.0001) groups, compared with the sham group. Moreover, the body weight significantly decreased in the TBI+vehicle group, compared with the TBI+2DG-D-Rosi group (p<0.01) (data not shown).

To evaluate the muscle strength and sensorimotor coordination, we compared the changes in the grip strength and rotarod performance before (baseline) and at 1-d post-treatment. For the grip strength test, upon two-way ANOVA analysis, there were significant differences based on treatment [F_{(3, 72)}=40.7, p<0.0001] and sex [F_{(1, 72)}=11.33, p=0.0012]. Specifically, grip strength significantly decreased in both male and female TBI+vehicle and TBI+Rosi groups, compared with the male and female sham and TBI+2DG-D-Rosi groups (data not shown). For the Rotarod test, upon two-way ANOVA analysis, there were significant differences based on treatment [F_{(3, 72)}=38.45, p<0.0001]. In male, the latency to the first fall significantly decreased in both TBI+vehicle (p<0.0001) and TBI+Rosi (p<0.0001) groups, compared with the sham group. In female, the latency to the first fall significantly decreased in TBI+vehicle (p<0.0001), TBI+Rosi (p<0.0001) and TBI+2DG-D-Rosi (p<0.001) groups, compared with the sham groups. In addition, the latency to the first fall significantly decreased in the TBI+vehicle (p<0.05) and TBI+Rosi (p<0.01) groups, compared with the TBI+2DG-D-Rosi group (data not shown).

Next, tail suspension and light/dark box tests were used to evaluate the anxiety and depression-like behaviors at 1-d post-treatment. For the tail suspension test, upon two-way ANOVA analysis, there were significant differences based on treatment [F_{(3, 72)}=42.62, p<0.0001]. Specifically, the duration of immobile significantly increased in both male and female TBI+vehicle and TBI+Rosi groups, compared with the male and female sham and TBI+2DG-D-Rosi groups. For the light/dark box test, upon two-way ANOVA analysis, there were significant differences in the time spent in the light compartment based on treatment [F_{(3, 72)}=5.09, p=0.0030]. Novel object recognition test was used to evaluate cognitive function at 1-d post-treatments. Upon two-way ANOVA analysis, there were significant differences in the discrimination index based on treatment [F_{(3, 72)}=12.32, p<0.0001]. Specifically, the mice in the male TBI+vehicle (p<0.0001) and TBI+Rosi (p<0.05) groups spent significantly less time with the novel object, compared with the male sham group. In addition, the mice in the male TBI+vehicle group (p<0.001) spent significantly less time with the novel object, compared with the male TBI+2DG-D-Rosi (data not shown).

These results demonstrated that 2DG-D-Rosi showed a better efficacy in improving behavioral outcomes, compared with the free Rosi. For example, the grip strength performance significantly decreased in both male and female vehicle-treated and Rosi-treated TBI animals, but not in the 2DG-D-Rosi treated group. Moreover, 2DG-D-Rosi treatment significantly improved grip strength, compared with free Rosi treatment in both males and females. In addition, 2DG-D-Rosi treatment significantly decreased immobile time during tail suspension test, compared with the TBI+saline and TBI+Rosi groups in both males and females. Whereas Rosi-treated TBI animals did not show significant improvement in any of the behavioral tests, compared with the vehicle-treated group.

### 2DG-D-Rosi Decreased Neuroinflammatory Responses and Cell Death

Previous studies have shown that Rosi exerts neuroprotective effects via the suppression of neuronal autophagy and apoptosis, regulates astrocyte polarization and attenuates neuroinflammation after TBI. However, in most of the studies, Rosi was administered immediately (˜5 min) after TBI and requires frequent re-dosing. In a focal cerebral ischemia mouse model, Rosi was given at 2 hours post-injury, however, only the dosage higher than 3 mg/kg can provide neuroprotection against brain infarcts. In the present study, we chose the 6-hour post-injury as the initial treatment time point, which reflects the clinical treatment time for TBI patients and provides the basis for assessment of therapeutic time window of the 2DG-D-Rosi platform.

Because 2DG-D-Rosi exhibits neuron-targeting properties, we isolated primary neurons from the injured brain regions (or the matching areas in the sham) to evaluate the efficacy of Rosi and 2DG-D-Rosi on neuro-inflammatory responses and cell death in neurons. We first compared the mRNA expression of pro-inflammatory markers (TNF-α, IL-1β, TLR4, and NLRP3) at 1-d post-treatments. Upon two-way ANOVA analysis [sex (male, female), treatment (sham, TBI+vehicle, TBI+Rosi, TBI+2DG-D-Rosi)], there were significant differences in the TNF-α expression based on treatment [F_{(3, 40)}=33.71, p<0.0001] and the interaction (treatment*sex) [F_{(3, 40)}=3.55, p=0.0227]. In males, TNF-α expression significantly increased in TBI+vehicle (p<0.001) and TBI+Rosi (p<0.05) groups, compared with the sham group. TNF-α expression significantly increased in the TBI+vehicle group, compared with the TBI+Rosi (p<0.05) and TBI+2DG-D-Rosi (p<0.001) groups. Moreover, TNF-α expression significantly increased in the TBI+Rosi group, compared with the TBI+2DG-D-Rosi group (p<0.001). In female, TNF-α expression significantly increased in TBI+vehicle (p<0.001), TBI+Rosi (p<0.001) and TBI+2DG-D-Rosi (p<0.01) groups, compared with the sham group. Moreover, TNF-α expression significantly increased in the TBI+vehicle (p<0.05) and TBI+Rosi (p<0.05) groups, compared with the TBI+2DG-D-Rosi group (data not shown). There were significant differences in the IL-1ß expression based on treatment [F_{(3, 40)}=11.66, p<0.0001], sex [F_{(1, 40)}=5.01, p=0.0308], and the interaction (treatment x sex) [F_{(3, 40)}=5.52, p=0.0029]. In males, IL-1ß expression significantly increased in the TBI+vehicle group, compared with sham (p<0.0001), TBI+Rosi (p<0.001) and TBI+2DG-D-Rosi (p<0.0001) groups. Moreover, IL-1ß expression significantly increased in the TBI+Rosi group, compared with the TBI+2DG-D-Rosi group (p<0.05). In females, IL-1β expression significantly increased in the TBI+vehicle (p<0.01) and TBI+Rosi (p<0.001) groups, compared with sham group. Moreover, IL-1ß expression significantly increased in the TBI+vehicle (p<0.01) and TBI+Rosi (p<0.001) groups, compared with the TBI+2DG-D-Rosi group (data not shown). There were significant differences in the TLR4 expression based on treatment [F_{(3, 40)}=16.33, p<0.0001], sex [F_{(1, 40)}=8.61, p=0.0055] and the interaction (treatment x sex) [F_{(3, 40)}=4.66, p=0.0069]. Specifically, TLR4 expression significantly increased in the male TBI+vehicle (p<0.001) and TBI+Rosi (p<0.05) groups, compared with the male sham group. Moreover, TLR4 expression significantly increased in the male TBI+vehicle (p<0.0001) and TBI+Rosi (p<0.001) groups, compared with the TBI+2DG-D-Rosi group (data not shown). There were significant differences in the NLRP3 expression based on treatment [F_{(3, 40)}=66.63, p<0.0001] and the interaction (treatment x sex) [F_{(3, 40)}=12.77, p<0.0001]. In males, NLRP3 expression significantly increased in the TBI+vehicle group, compared with the male sham (p<0.0001), TBI+Rosi (p<0.0001), and TBI+2DG-D-Rosi (p<0.0001) groups. Moreover, NLRP3 expression significantly increased in the TBI+Rosi (p<0.05) group, compared with the TBI+2DG-D-Rosi group. In females, NLRP3 expression significantly increased in the TBI+vehicle (p<0.0001) and TBI+Rosi (p<0.001) groups, compared with the sham group. Moreover, NLRP3 expression significantly increased in the TBI+vehicle (p<0.001) and TBI+Rosi (p<0.01) groups, compared with the TBI+2DG-D-Rosi group (data not shown).

Next, we compared the mRNA expression of anti-inflammatory marker IL-13 at 1-d post-treatments. Upon two-way ANOVA analysis, there were significant differences in the IL-13 expression based on treatment [F_{(3, 40)}=17.19, p<0.0001]. In males, IL-13 expression significantly decreased in TBI+vehicle group, compared with the sham (p<0.01) and TBI+2DG-D-Rosi (p<0.01) groups. In females, IL-13 expression significantly decreased in TBI+vehicle (p<0.001) and TBI+Rosi (p<0.01) groups, compared with the sham group (data not shown).

We further compared the mRNA expression of cell death marker Fas at 1-d post-treatments. Upon two-way ANOVA analysis, there were significant differences in the Fas expression based on treatment [F_{(3,40)}=36.24, p<0.0001], sex [F_{(1, 40)}=9.56, p=0.0036], and the interaction (treatment x sex) [F_{(3, 40)}=13.71, p<0.0001]. In males, Fas expression significantly increased in the TBI+vehicle group, compared with the sham (p<0.0001), TBI+Rosi (p<0.0001), and TBI+2DG-D-Rosi (p<0.0001) groups. In females, Fas expression significantly increased in the TBI+vehicle (p<0.001), TBI+Rosi (p<0.001) and TBI+2DG-D-Rosi (p<0.05) groups, compared with the sham group. Moreover, Fas expression significantly increased in the TBI+Rosi group, compared with the TBI+2DG-D-Rosi (p<0.01) groups (data not shown).

Because 2DG-D-Rosi treatment significantly decreased the cell death markers, next, we used the Fluoro-Jade C (FJC) staining to evaluate the neuroprotective effects of 2DG-D-Rosi. Upon two-way ANOVA analysis, there were significant differences in FJC+NeuN⁺ cells based on treatment [F_{(3, 24)}=126.91, p<0.0001]. In males, FJC+NeuN⁺ cells significantly increased in the TBI+vehicle (p<0.0001), TBI+Rosi (p<0.0001) and TBI+2DG-D-Rosi (p<0.0001) groups, compared with the sham group. FJC+NeuN⁺ cells significantly increased in the TBI+vehicle, compared with the TBI+Rosi (p<0.05) and TBI+2DG-D-Rosi (p<0.0001) groups. Moreover, FJC+NeuN⁺ cells significantly increased in the TBI+Rosi (p<0.05) group, compared with the TBI+2DG-D-Rosi group. In females, FJC+NeuN⁺ cells significantly increased in the TBI+vehicle (p<0.0001), TBI+Rosi (p<0.0001) and TBI+2DG-D-Rosi (p<0.0001) groups, compared with the sham group. Moreover, FJC+NeuN⁺ cells significantly increased in TBI+vehicle (p<0.01) and TBI+Rosi (p<0.05) groups, compared with the TBI+2DG-D-Rosi group.

Our in vivo results demonstrate that the 2DG-D-Rosi treatment significantly decreased pro-inflammatory markers, such as TNF-α, IL-1ß and NLRP3 in both males and females, compared with the free Rosi treatment. Studies have shown that pediatric TBI increase production of proinflammatory cytokines such as TNF-α and IL-1β, which can exacerbate lipid peroxidation and ionic imbalance, promotes cell necrosis and accelerates neurodegenerative processes. NLRP3 inflammasome plays an important role in the primary inflammatory response to TBI. NLRP3 activation promotes proinflammatory cytokines, such as IL-1ß and IL-18, which promote the accumulation of ROS and worsen neuroinflammation and oxidative stress, leading to cell death. The significant anti-inflammatory effects of 2DG-D-Rosi can be responsible for the decreased cell death marker and cell loss.

Interestingly, there are sex differences in the behavioral outcomes and the expression of inflammatory and cell death markers among treatment groups. Studies have shown that there are sex differences in the pharmacokinetics and efficacy of rosiglitazone. For example, Rosi can stimulate fat growth and improve insulin sensitivity in female mice, but not in males. Moreover, mounting evidence indicates that TBI alone can induce sex-specific neuroinflammatory responses and behavioral outcomes, depending on differential cellular responses, sex hormones, and metabolism. Therefore, the sex differences in behaviors and neuroinflammatory responses can be caused by the combined effects of brain injury and drug treatment, however, the underlying mechanisms need to be further investigated.

### Conclusions

We aimed to develop an effective neuron-targeted delivery system for Rosi for targeted treatment of TBI. Despite its potential as a therapeutic agent, Rosi faces significant limitations in TBI treatment, including poor solubility, systemic side effects, difficulty in crossing the BBB, and targeted delivery to neurons specifically at the site of brain injury. The 2DG-D-Rosi delivery platform effectively overcame the challenges of Rosi's poor solubility, and limited brain bioavailability. 2DG-D-Rosi demonstrated enhanced neuroprotective effects compared to free Rosi in vitro under TBI like conditions. The in vivo results demonstrated that by leveraging the dendrimer's targeting capabilities, 2DG-D-Rosi achieved precise delivery to injured neurons, reducing neuroinflammation and neuronal loss while improving functional outcomes in a pediatric TBI model. The 2DG-D-Rosi nanotherapy and 2DG-D nanoplatform offer a useful treatment strategy for TBI, with applications in other neurodegenerative conditions requiring targeted drug delivery to neurons across the BBB.

### Example 3

In this Example, we present the design and development of 2DG-D-TPP (the mitochondria-targeting moiety triphenylphosphonium (TPP) conjugated onto the 2DG-surfaced dendrimer (2DG-D) scaffold) and its Pio conjugate (2DG-D-TPP-Pio). The targeting and therapeutic efficacy of the constructs is evaluated in vivo using a pediatric traumatic brain injury (TBI) mouse model, presenting a new strategy for enhanced neuroprotection in neurodegenerative diseases.

### Experimental Section

### Material and Reagents

All starting materials and reagents were obtained from Sigma-Aldrich and Merck. Analytical-grade reagents and solvents used in the synthesis were used as received. Reactions requiring anhydrous conditions were conducted under a positive nitrogen atmosphere, with all glassware dried in an oven beforehand. Analytical thin-layer chromatography (TLC) was performed on silica gel 60 F₂₅₄ aluminum-backed plates, and spots were visualized using UV light or staining reagents. Compounds were purified by flash column chromatography on silica gel 60 (230-400 mesh). Dialysis membranes (Spectra/Por) were sourced from Repligen.

### Instruments

Nuclear Magnetic Resonance (NMR) spectra were acquired using a Bruker 500 MHz high-resolution NMR spectrometer, with samples prepared in deuterated solvents such as chloroform (CDCl₃), deuterated DMSO (DMSO-d₆), or deuterated water (D₂O). Chemical shifts for ¹H-NMR are given in parts per million (ppm) and are referenced to the solvent peak. Coupling constants (J) are reported in hertz (Hz), with the following abbreviations used to describe signal patterns: s=singlet, d=doublet, t=triplet, q=quartet, and m=multiplet. High resolution mass spectrometry (HRMS) spectra were acquired using a Waters Q-Tof Premier mass spectrometer operating in positive-ion, V-mode using electrospray ionization.

High-performance liquid chromatography (HPLC) was used to assess the purity of small molecules, as well as dendrimers and dendrimer-drug conjugates, and to evaluate drug release studies. Analyses were performed on a Waters Acquity Arc® system (Milford, MA, USA) equipped with binary pumps, a 2998 PDA detector, and a 2475 fluorescence detector, using Waters Empower® software. Samples were analyzed on a Waters C18 Symmetry 300 column (5 μm, 4.6×250 mm) with a gradient flow method. The gradient started at 90:10 (Solvent A: 0.1% TFA and 5% ACN in water; Solvent B: 0.1% TFA in ACN), gradually increasing to 50:50 (A:B) at 20 minutes, 10:90 (A:B) at 38 minutes finally returning to 90:10 (A:B) at 45 minutes, with a flow rate of 1 mL/min. Dendrimers and drug conjugates were monitored at 254 and 269 nm, while Cy5 conjugates were monitored at 650 nm.

### Synthetic Procedures

Synthesis of TPP-Azide (3): To a stirred solution of TPP-Acid (1) (860 mg, 2.0 mmol, 1.0 equiv) in anhydrous DMF (5 mL), HOBt (367.5 mg, 2.4 mmol, 1.2 equiv), DIPEA (0.5 mL, 3.0 mmol, 1.5 equiv), and HBTU (910 mg, 2.4 mmol, 1.2 equiv) were sequentially added at room temperature. Subsequently, azido-PEG5-amine (2) (1.2 g, 4.0 mmol, 2.0 equiv) was added to the reaction mixture. The reaction was stirred at ambient temperature for 12 hours, and the progress was monitored by thin-layer chromatography (TLC) under UV light. Upon completion, the reaction mixture was diluted with dichloromethane (DCM), and the organic phase was washed with water (5×30 mL) and brine (1 × 30 mL). The combined organic layers were dried over anhydrous sodium sulfate (Na₂SO₄), filtered, and concentrated under reduced pressure. The crude product was purified by silica gel column chromatography using ethyl acetate/hexanes (70:30, v/v) as the eluent to yield TPP-Azide (3) as a white solid in 77% yield.

HPLC purity: 97.69%, retention time: 19.252 minutes.

¹H NMR (500 MHZ, DMSO) δ 8.06 (t, J=5.6 Hz, 1H, Amide H), 7.94 (m, 4.4 Hz, 3H, Aromatic H), 7.83 (m, 12H, Aromatic H), 3.64-3.52 (m, 20H, PEG H), 3.44-3.42 (m, 4H, PEG H), 3.24 (q, J=5.8 Hz, 2H,—CH₂), 2.37 (t, J=7.1 Hz, 2H,—CH₂), 1.76 (m, 2H,—CH₂).

¹³C NMR (125 MHz, DMSO) δ 171.18, 162.77, 135.43, 135.40, 134.09, 134.00, 130.79, 130.69, 119.23, 118.55, 70.28, 70.25, 70.22, 70.18, 70.15, 70.01, 69.71, 69.55, 54.06, 50.44, 42.31, 39.04, 38.70, 36.23, 35.35, 35.21, 20.66, 20.25, 18.70, 18.67, 18.53, 17.17, 12.96. m/z: calculated for C₃₄H₄₇N₄O₆P [M+H]⁺: 637.3149; found 637.3243.

Synthesis of 2DG-D-Hexyne (6): EDC. HCl (31 mg, 18.0 eq, 0.16 mmol) was added to a solution of hexynoic acid (5) (9.2 mg, 9.0 eq, 0.081 mmol) in anhydrous DMF (5 mL) and stirred for 15 minutes. The resulting solution was then added dropwise to a solution of 2DG dendrimer (4) (200 mg, 1.0 eq, 0.009 mmol) in DMF (5 mL) under continuous stirring, followed by the addition of DMAP (5.5 mg, 5.0 eq, 0.045 mmol). The reaction mixture was stirred at room temperature for 24 hours. Completion of the reaction was confirmed by a shift in the retention time in the HPLC chromatogram. The crude product was purified by dialysis using a 1 kDa membrane in DMF for 12 hours, followed by 12-hour dialysis in deionized water. The purified product was lyophilized to afford compound 6 in 80% yield.

HPLC purity: 99.07%, retention time: 18.012 minutes.

¹H NMR (500 MHz, DMSO) δ 8.50 (m, 8H, Amide H), 8.08-7.85 (m, 52H, Amide and Triazole H), 7.22 (s, 16H, Aromatic H), 5.22-5.13 (m, 6H, Sugar H), 4.95-4.82 (m, 32H, Sugar H), 4.77 (m, 9H, Sugar H), 4.61-4.42 (m, 130H, Sugar H), 4.22-4.04 (m, 64H, Sugar H), 3.87-3.78 (m, 109H, Sugar and PEG H), 3.72-3.28 (m, PEG H), 3.19-3.00 (m, 68H, Core and PEG H), 2.83 (m, 11H, Core H), 2.70 (m, 19H, Core H), 2.62-2.60 (m, 22H, Core H), 2.46-2.42 (m, 14H, Hexyne H), 2.30-2.09 (m, 46H, Core and Hexyne H), 1.94-1.82 (m, 40H, Core and Sugar H), 1.76-1.71 (m, 14H, Hexyne H), 1.52-1.44 (m, 16H, Sugar H).

¹³C NMR (125 MHz, DMSO) δ 152.23, 144.24, 124.71, 122.68, 106.70, 97.13, 73.56, 72.32, 72.18, 72.12, 70.41, 70.30, 70.25, 70.23, 70.21, 70.14, 70.07, 69.99, 69.49, 69.41, 69.38, 69.23, 69.16, 68.81, 68.42, 66.24, 66.05, 64.15, 63.96, 61.48, 49.76, 49.63, 38.79, 38.33, 35.31, 25.61, 25.11, 17.53.

Synthesis of 2DG-D-TPP (7): To a stirred solution of 2DG-D-Hexyne (6) (500 mg, 1.0 eq, 0.02 mmol) in deionized water (1 mL) in a microwave vial was added a solution of TPP-Azide (3) (74.5 mg, 5.5 eq, 0.11 mmol) dissolved in DMF (1 mL). It was followed by the addition of CuSO₄.5H₂O (10 mol % per alkyne) dissolved in deionized water (0.1 mL). After 2 minutes, sodium ascorbate (15 mol % per alkyne) was added, and the reaction mixture was stirred at 40° C. for 10 hours. Upon completion, the crude product was purified by dialysis using a 1 kDa membrane in DMF for 12 hours, followed by 12-hour dialysis in deionized water. The aqueous solution was lyophilized to afford compound 7 in 86% yield.

HPLC purity: 96.00%, retention time: 20.678 minutes.

¹H NMR (500 MHZ, DMSO) δ 8.51 (m, 8H, Dendrimer-Amide H), 8.14-7.77 (m, 132H, Dendrimer-Amide and Triazole H plus TPP-Amide and Aromatic H), 7.22 (s, 16H, Dendrimer-Aromatic H), 5.17-5.18 (m, 6H, Dendrimer-Sugar H), 4.97-4.82 (m, 38H, Dendrimer-Sugar H), 4.78-4.77 (m, 9H, Dendrimer-Sugar H), 4.65-4.39 (m, 133H, Dendrimer-Sugar H), 4.21-4.05 (m, 64H, Dendrimer-Sugar H), 3.91-3.74 (m, 114H, Dendrimer-Sugar and PEG H), 3.72-3.34 (m, Dendrimer plus TPP-PEG H), 3.25-3.21 (m, 15H, Dendrimer-PEG H plus TPP-CH₂), 3.12-3.04 (m, 31H, Dendrimer-Core H and Hexyne

H), 2.67-2.64 (m, 17H, Dendrimer-Core H and Hexyne H), 2.44-2.35 (m, 46H, Dendrimer-Core plus TPP-CH₂), 2.27-2.11 (m, 32H, Dendrimer-Core and Hexyne H), 1.95-1.67 (m, 68H, Dendrimer-Core and Sugar H plus TPP-CH₂), 1.50-1.46 (m, 16H, Dendrimer-Sugar H).

¹³C NMR (125 MHz, DMSO) δ 172.75, 171.16, 170.05, 166.01, 152.22, 144.23, 134.08, 134.00, 130.78, 130.68, 124.71, 118.53, 106.70, 97.22, 97.12, 73.56, 72.32, 72.18, 72.11, 70.40, 70.29, 70.25, 70.21, 70.13, 70.07, 69.98, 69.40, 69.22, 69.16, 68.80, 68.42, 66.04, 63.95, 61.47, 49.75, 38.33, 25.61, 23.97, 17.53.

Synthesis of 2DG-D-TPP-Cy5 (9): To a stirred solution of 2DG-D-TPP (7) (30 mg, 1.0 eq, 0.00117 mmol) in deionized water (1 mL) in a microwave vial was added a solution of Cy5-Azide (8) (2.9 mg, 2.5 eq, 0.0029 mmol) dissolved in DMF (1 mL). It was followed by the addition of CuSO₄.5H₂O (10 mol % per alkyne) dissolved in deionized water (0.1 mL). After 2 minutes, sodium ascorbate (15 mol % per alkyne) was added, and the reaction mixture was stirred at 40° C. for 10 hours. Upon completion, the crude product was purified by the dialysis using a 1 kDa dialysis membrane in DMF for 12 hours, followed by 12-hour dialysis in deionized water. The aqueous solution was lyophilized to afford compound 9 in 83% yield.

HPLC purity: 98.31%, retention time: 20.048 minutes.

¹H NMR (500 MHZ, DMSO) δ 8.51 (m, 8H, Dendrimer-Amide H), 8.40 (m, 8H, Cy5-Aromatic H), 8.26 (m, 2H, Cy5-Aromatic H), 8.14-7.74 (m, 141H, Dendrimer-Amide and Triazole H plus TPP-Amide and Aromatic H plus Cy5-Aromatic H), 7.68 (m, 4H, Cy5-Aromatic H), 7.35 (m, 4H, Cy5-Aromatic H), 7.22 (s, 16H, Dendrimer-Aromatic H), 6.60 (m, 6H, Cy5 H), 6.34 (m, 6H, Cy5 H), 5.19 (m, 13H, Dendrimer-Sugar H plus Cy5 H), 4.99-4.82 (m, 40H, Dendrimer-Sugar H), 4.77 (m, 8H, Dendrimer-Sugar H), 4.51 (m, 132H, Dendrimer-Sugar H), 4.36-4.28 (m, 19H, Dendrimer-Sugar H plus Cy5 H), 4.12 (m, 64H, Dendrimer-Sugar H), 3.81 (m, 114H, Dendrimer-Sugar and PEG H), 3.73-3.45 (m, Dendrimer plus TPP-PEG H), 3.28-3.19 (m, 20H, Dendrimer-PEG H plus TPP-CH₂), 3.15-2.96 (m, 41H, Dendrimer-Core H), 2.75-2.62 (m, 42H, Dendrimer-Core H plus Cy5—CH₂), 2.45-2.32 (m, 37H, Dendrimer-Core and Hexyne H plus TPP-CH₂), 2.28-2.04 (m, 39H, Dendrimer-Core and Hexyne H), 1.97-1.65 (m, 101H, Dendrimer-Core and Sugar H plus TPP-CH₂ plus Cy5-CH₂), 1.61-1.40 (m, 31H, Dendrimer-Sugar H plus Cy5—CH₂), 1.39-1.22 (m, 29H, Cy5-CH₂).

Synthesis of 2DG-D-Hexyne′ (10): EDC. HCl (54 mg, 32.0 eq, 0.28 mmol) was added to a solution of hexynoic acid (5) (16.1 mg, 16.0 eq, 0.144 mmol) in anhydrous DMF (5 mL) and stirred for 15 minutes. The resulting solution was then added dropwise to a solution of 2DG dendrimer (4) (200 mg, 1.0 eq, 0.009 mmol) in DMF (5 mL) under continuous stirring, followed by the addition of DMAP (8.7 mg, 8.0 eq, 0.072 mmol). The reaction mixture was stirred at room temperature for 24 hours. Completion of the reaction was confirmed by a shift in the retention time in the HPLC chromatogram. The crude product was purified by dialysis using a 1 kDa membrane in DMF for 12 hours, followed by 12-hour dialysis in deionized water. The purified product was lyophilized to afford compound 10 in 80% yield.

HPLC purity: 99.84%, retention time: 19.667 minutes.

¹H NMR (500 MHz, DMSO) δ 8.50 (m, 8H, Amide H), 8.07-7.84 (m, 52H, Amide and Triazole H), 7.21 (s, 16H, Aromatic H), 5.18-5.16 (m, 6H, Sugar H), 4.99-4.84 (m, 40H, Sugar H), 4.77-4.76 (m, 9H, Sugar H), 4.55-4.44 (m, 133H, Sugar H), 4.16-4.07 (m, 64H, Sugar H), 3.84-3.77 (m, 114H, Sugar and PEG H), 3.70-3.48 (m, PEG H), 3.17-3.01 (m, 68H, Core H and PEG H), 2.83 (m, 12H, Core H), 2.70 (m, 19H, Core H), 2.63-2.60 (m, 22H, Core H), 2.45-2.42 (m, 32H, Hexyne H), 2.29-2.08 (m, 62H, Core and Hexyne H), 1.95-1.81 (m, 40H, Core and Sugar H), 1.74-1.70 (m, 32H, Hexyne H), 1.49-1.43 (m, 16H, Sugar H).

¹³C NMR (125 MHz, DMSO) & 162.78, 152.22, 144.24, 124.71, 106.71, 97.12, 73.56, 72.32, 72.18, 72.11, 72.05, 70.40, 70.29, 70.25, 70.23, 70.21, 70.13, 70.07, 69.98, 69.49, 69.41, 69.38, 69.16, 68.81, 68.42, 66.05, 64.15, 63.96, 61.47, 49.75, 38.33, 37.42, 36.25, 34.56, 31.23, 25.61, 23.97, 17.53, 16.15.

Synthesis of 2DG-D-TPP′ (11): To a stirred solution of 2DG-D-Hexyne′ (10) (500 mg, 1.0 eq, 0.02 mmol) in deionized water (1 mL) in a microwave vial was added a solution of TPP-Azide (3) (74.5 mg, 5.5 eq, 0.11 mmol) dissolved in DMF (1 mL). It was followed by the addition of CuSO₄.5H₂O (10 mol % per alkyne) dissolved in deionized water (0.1 mL). After 2 minutes, sodium ascorbate (15 mol % per alkyne) was added, and the reaction mixture was stirred at 40° C. for 10 hours. Upon completion, the crude product was purified by dialysis using a 1 kDa membrane in DMF for 12 hours, followed by 12-hour dialysis in deionized water. The aqueous solution was lyophilized to afford compound 11 in 85% yield. HPLC purity: 97.79%, retention time: 21.963 minutes.

¹H NMR (500 MHZ, DMSO) & 8.50 (m, 8H, Dendrimer-Amide H), 8.14-7.71 (m, 132H, Dendrimer-Amide and Triazole H plus TPP-Amide and Aromatic H), 7.21 (s, 16H, Dendrimer-Aromatic H), 5.18-5.17 (m, 7H, Dendrimer-Sugar H), 4.92-4.83 (m, 39H, Dendrimer-Sugar H), 4.78-4.76 (m, 8H, Dendrimer-Sugar H), 4.56-4.45 (m, 135H, Dendrimer-Sugar H), 4.22-4.05 (m, 65H, Dendrimer-Sugar H), 3.85-3.77 (m, 125H, Dendrimer-Sugar and PEG H), 3.74-3.46 (m, Dendrimer plus TPP-PEG H), 3.25-3.20 (m, 14H, Dendrimer-PEG H plus TPP-CH₂), 3.15-3.03 (m, 48H, Dendrimer-Core H and Hexyne H), 2.68-2.60 (m, 31H, Dendrimer-Core H and Hexyne H), 2.48-2.34 (m, 46H, Dendrimer-Core plus TPP-CH₂), 2.28-2.09 (m, 50H, Dendrimer-Core and Hexyne H), 1.97-1.65 (m, 68H, Dendrimer-Core and Sugar H plus TPP-CH₂), 1.68-1.32 (m, 20H, Dendrimer-Sugar H).

¹³C NMR (126 MHZ, DMSO) δ 172.75, 171.16, 170.05, 166.01, 152.22, 144.23, 134.08, 134.00, 130.78, 130.68, 124.71, 118.53, 106.70, 106.70, 99.76, 97.22, 97.12, 73.56, 72.32, 72.18, 72.11, 70.40, 70.29, 70.25, 70.21, 70.13, 70.07, 69.98, 69.40, 69.22, 69.16, 68.80, 68.42, 66.04, 63.95, 61.47, 52.37, 49.75, 38.33, 25.61, 23.97, 17.53.

Synthesis of 2DG-D-TPP-Pio (13): To a stirred solution of 2DG-D-TPP′ (11) (300 mg, 1.0 eq, 0.0116 mmol) in 3 mL DI water was added solution of Pio Azide (12) (70.5 mg, 11.5 eq, 0.134 mmol) dissolved in 1 mL DMF. It was followed by the addition of CuSO₄.5H₂O (10 mol % per acetylene) dissolved in 0.1 mL DI water. After 2 minutes, sodium ascorbate (15 mol % per acetylene) was added and the reaction was stirred at room temperature for 12 hours. Upon completion, the dialysis was performed using a 1 kDa dialysis membrane in DMF for 12 hours, followed by 12-hour dialysis in deionized water. The aqueous solution was lyophilized to afford compound 13 in 88% yield.

HPLC purity: 99.07%, retention time: 22.967 minutes.

¹H NMR (500 MHZ, DMSO) δ 8.52 (m, 8H, Dendrimer-Amide H), 8.39 (m, 11H, Pio-Aromatic H), 8.11-7.76 (m, 132H, Dendrimer-Amide and Triazole H plus TPP-Amide and Aromatic H), 7.60-7.58 (m, 11H, Pio-Aromatic H), 7.30-7.29 (m, 11H, Pio-Aromatic H), 7.22 (s, 16H, Aromatic H), 7.13-7.11 (m, 22H, Pio-Aromatic H), 6.85-6.84 (m, 22H, Pio-Aromatic H), 4.99-4.85 (m, 49H, Dendrimer-Sugar H), 4.61-4.41 (m, 180H, Dendrimer-Sugar H plus Pio-Aliphatic H), 4.33-4.28 (m, 50H, Dendrimer-Sugar H plus Pio-Aliphatic H), 4.19-4.05 (m, 66H, Dendrimer-Sugar H), 3.88-7.76 (m, 122H, Dendrimer-Sugar and PEG H), 3.69-3.49 (m, Dendrimer plus TPP-PEG H), 3.19-3.04 (m, 129H, Dendrimer-Core and Hexyne H), 2.69-2.59 (m, 99H, Dendrimer-Core H plus Pio-Aliphatic H), 2.42-2.34 (s, 53H, Dendrimer-Core and Hexyne H plus TPP-CH₂), 2.31-2.12 (m, 69H, Dendrimer-Core and Hexyne H plus Pio-Aliphatic H), 1.92-1.68 (m, 106H, Dendrimer-Core and Sugar H plus TPP-CH₂ plus Pio-Aliphatic H), 1.58-1.41 (m, 52H, Dendrimer-Sugar H plus Pio-Aromatic H), 1.36-1.13 (m, 81H, Pio-Aliphatic H).

¹³C NMR (125 MHz, DMSO) δ 172.78, 149.02, 146.46, 144.23, 136.15, 131.77, 124.70, 123.46, 114.11, 106.70, 97.22, 97.13, 73.56, 72.32, 72.11, 70.40, 70.29, 70.25, 70.20, 70.13, 70.07, 69.98, 69.40, 69.23, 69.16, 68.80, 68.42, 68.22, 67.04, 66.05, 63.96, 61.47, 49.74, 49.45, 38.79, 38.33, 37.92, 37.29, 35.31, 33.82, 29.91, 25.74, 25.61, 25.43, 25.11, 24.81, 24.29, 21.90, 15.86.

In Vivo mice

Male and female C57BL/6 mice were randomized at postnatal day 20-21 into sham, traumatic brain injury (TBI)+vehicle, TBI+Pioglitazone (TBI+Pio), TBI+2DG-D-Pioglitazone (TBI+D-Pio), TBI+2DG-D-TTP-Pioglitazone (TBI+D-TTP-Pio), and TBI+D-TTP-Pio-Cy5 groups. The TBI groups underwent TBI procedure, and the sham group underwent anesthesia without injury. The TBI groups received intraperitoneal administration of free pioglitazone (5 mg/kg, 100 μL), dendrimer-Pioglitazone (containing 5 mg/kg pioglitazone, 100 μL), dendrimer-TTP-Pioglitazone (containing 5 mg/kg pioglitazone, 100 μL), dendrimer-TTP-Pio-Cy5 (containing 50 mg/kg Cy5, 100 μL), or vehicle (100 μL) at 6-h post-injury. The mice from the sham group did not receive any intervention. Body weight and behavioral tests were measured before injury (baseline) and at 1-day (d) post-treatment. Animals were euthanized after behavioral testing, and the mitochondria of the neurons at the site of injury (or matching brain region in the sham animals) were isolated. The isolated mitochondria were further processed for RNA isolation and real-time quantitative PCR (qPCR) evaluations.

### Results and Discussion

### Synthesis and Characterization of 2DG-D-TPP Conjugate Using Click Chemistry

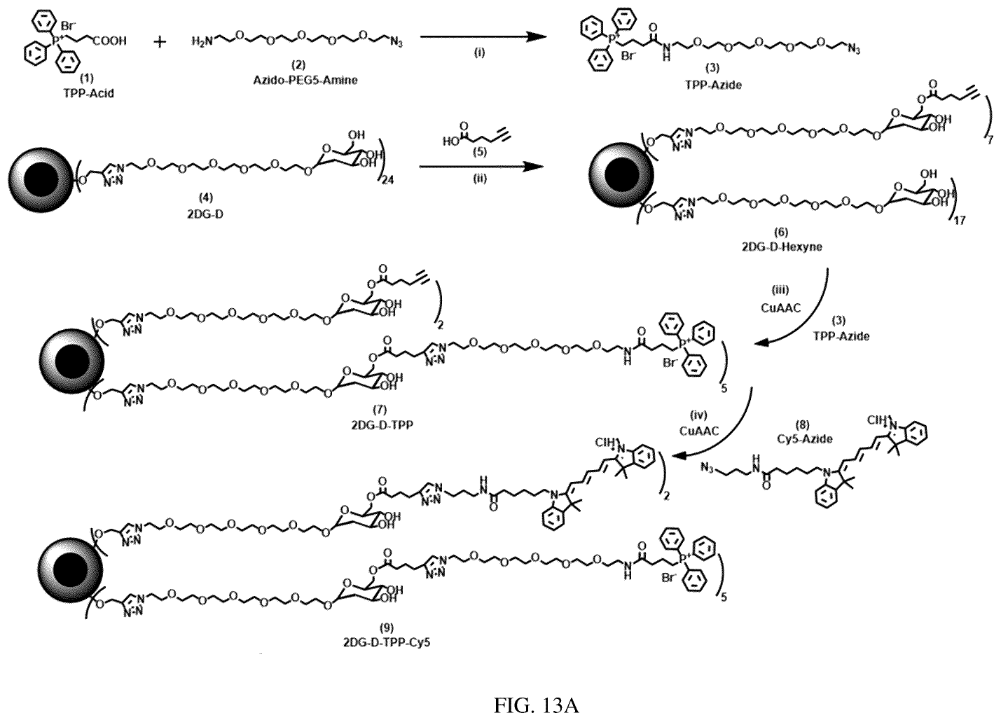

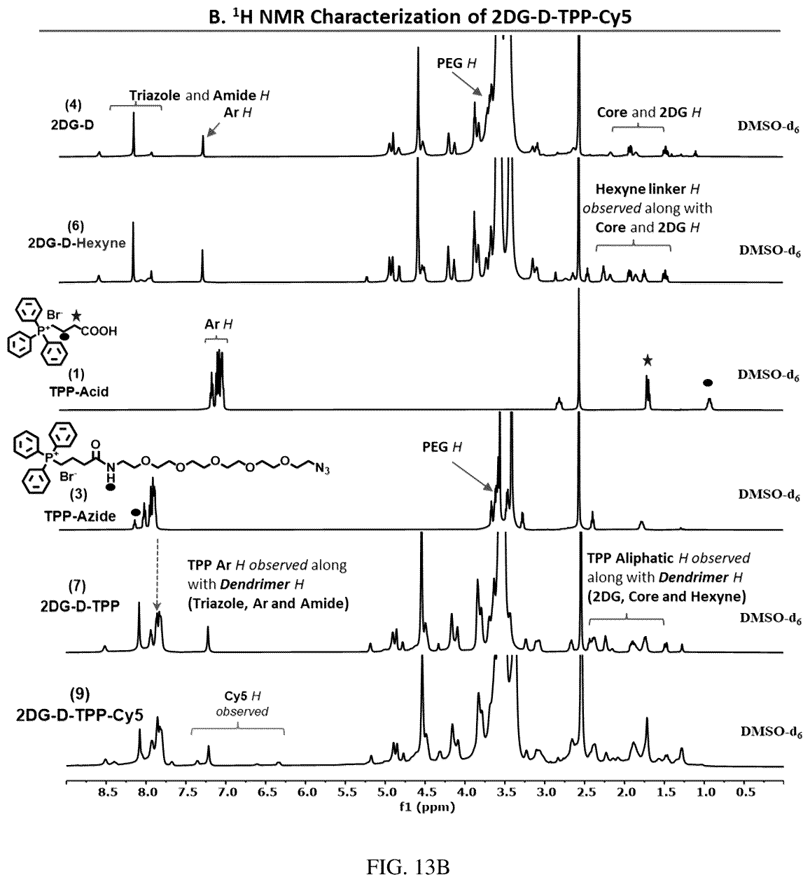

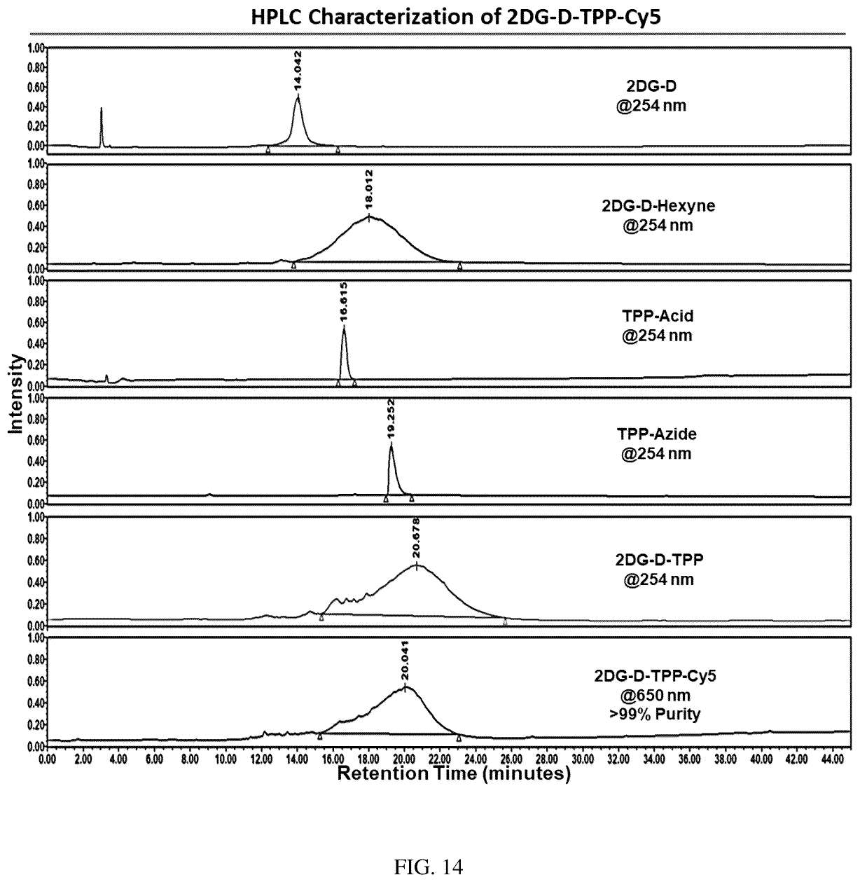

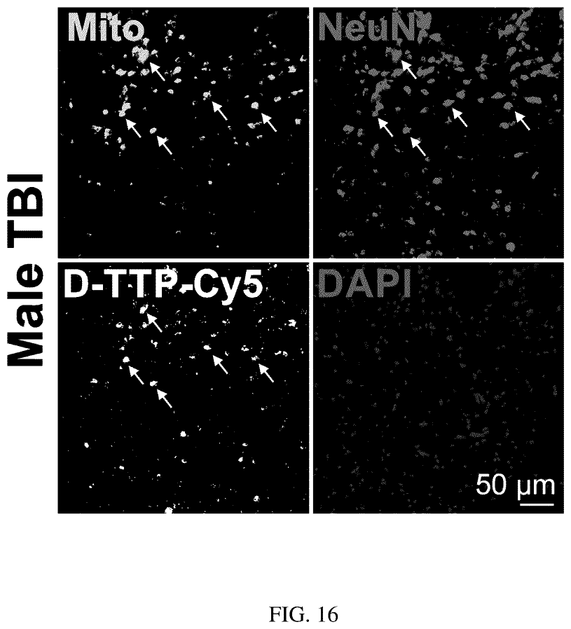

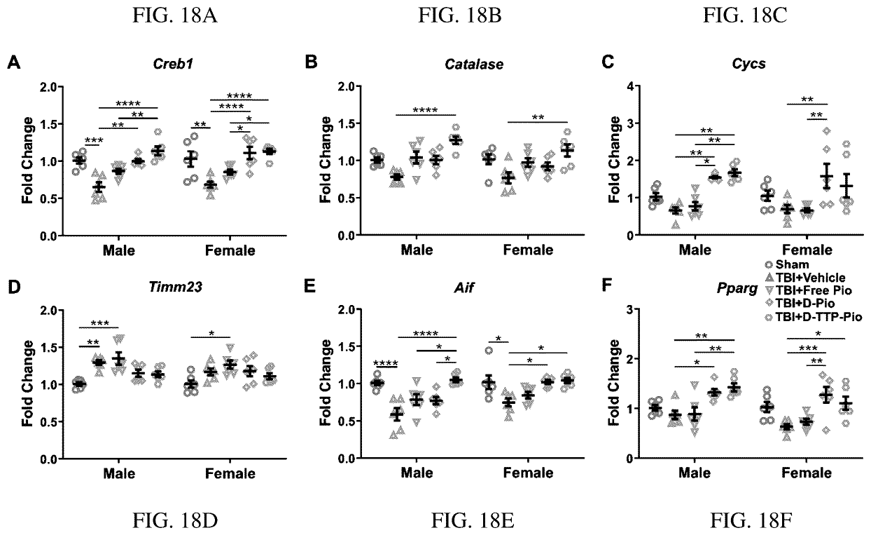

The 2DG-D-TPP conjugate was synthesized via a copper-catalyzed azide-alkyne cycloaddition (CuAAC) reaction. The CuAAC “click” reaction is a highly efficient and orthogonal coupling method that allows for the site-specific attachment of targeting ligands and drug molecules to nanomaterials, independent of other functional groups present. This strategy provides a reliable and versatile approach for the construction of dendrimer-based drug delivery systems. An azide group was first introduced onto the TPP moiety to enable the CuAAC reaction. TPP-Acid (1) was reacted with azido-PEG5-amine (2) in the presence of HOBt, HBTU and DIPEA to yield TPP-Azide (3) (FIG. 13A). The conjugation of the azide linker was confirmed through the ¹H NMR spectrum, which displayed PEG protons in between 83.2 and 83.6 ppm (FIG. 13B and FIG. 16). HPLC analysis showed a shift in retention time from 16.6 minutes for TPP-Acid (1) to 19.2 minutes for TPP-Azide (3), with more than 97% purity (FIG. 14 and FIG. 18). Next, we introduced alkynes on 2DG-D to facilitate the click reaction. We modified ˜7 hydroxyl groups on the surface of 2DG-D (4) by reacting with 5-hexynoic acid (5) in the presence of EDC-DMAP to afford 2DG-D-Hexyne (6) (FIG. 13A). The ¹H NMR showed the methylene protons from the hexynoic linker in the aliphatic region of dendrimer at 81.7 ppm (14H), between 82.1 and 82.2 ppm and at 82.4 ppm (14H) (FIG. 13B). The HPLC chromatogram showed a shift in retention time from 14.0 minutes for 2DG-D (4) to 18.0 minutes for 2DG-D-Hexyne (6) (FIG. 14). Subsequently, we conjugated TPP-Azide (3) and 2DG-D-Hexyne (6) using CuAAC to synthesize the neuronal mitochondria targeting platform 2DG-D-TPP (7). We hypothesized that attaching 5 TPP molecules (˜7% of the 72 hydroxyl surface groups) on the dendrimer would preserve its transport properties and maintain its inherent targeting capability for accumulation in neurons. Given TPP's high hydrophobicity, attaching more than this number could potentially reduce the dendrimer's aqueous solubility. Recent studies have also indicated that conjugating approximately 5 TPP moieties to dendrimers yields the optimal balance for effective mitochondrial targeting. CuAAC ensured high conjugation efficiency and enabled the desired loading of TPP ligand on the dendrimer. The shift in retention time of HPLC chromatogram from 18.0 minutes for 2DG-D-Hexyne (6) to 20.7 minutes for 2DG-D-TPP (7) suggested the completion of the reaction (FIG. 14). The number of attached TPP molecules was confirmed through the comparative integration of aromatic protons from the dendrimer at 87.2 ppm (16H), and the aromatic and amide protons of the ligand (16*5=80H) along with triazole and amide protons from the dendrimer (52H) between 87.8 and 88.1 ppm (80+52=132H). The HPLC purity level of 2DG-D-TPP (7) was found to be 96%. To investigate the targeting of neuronal mitochondria by 2DG-D-TPP using confocal microscopy and fluorescence spectroscopy, we further functionalized it by a near-infrared dye cyanine 5 (Cy5). The 2DG-D-TPP with ˜ 2 acetylene groups on its surface was reacted with Cy5-Azide (8) using CuAAC reaction to obtain fluorescently labeled 2DG-D-TPP-Cy5 (9). The successful attachment of Cy5 was confirmed by the presence of Cy5 protons in the ¹H NMR spectrum (FIG. 13B). The number of attached Cy5 molecules on the dendrimer surface was calculated using the proton integration method. Aromatic protons from the dendrimer at 87.2 ppm (16H), on comparison with Cy5 protons at 86.4 ppm (6H), 86.6 ppm (6H), 87.4 ppm (4H) and at 87.7 ppm (4H) suggested the attachment of ˜2 molecules of the Cy5 (FIG. 13B). The purity of 2DG-D-TPP-Cy5 was found to be >98% by HPLC, and the chromatogram showed a shift in the retention time from 20.7 to 20.0 minutes upon conjugation of Cy5 (FIG. 14).

### Synthesis and Characterization of 2DG-D-TPP-Pio Conjugate for therapy of TBI

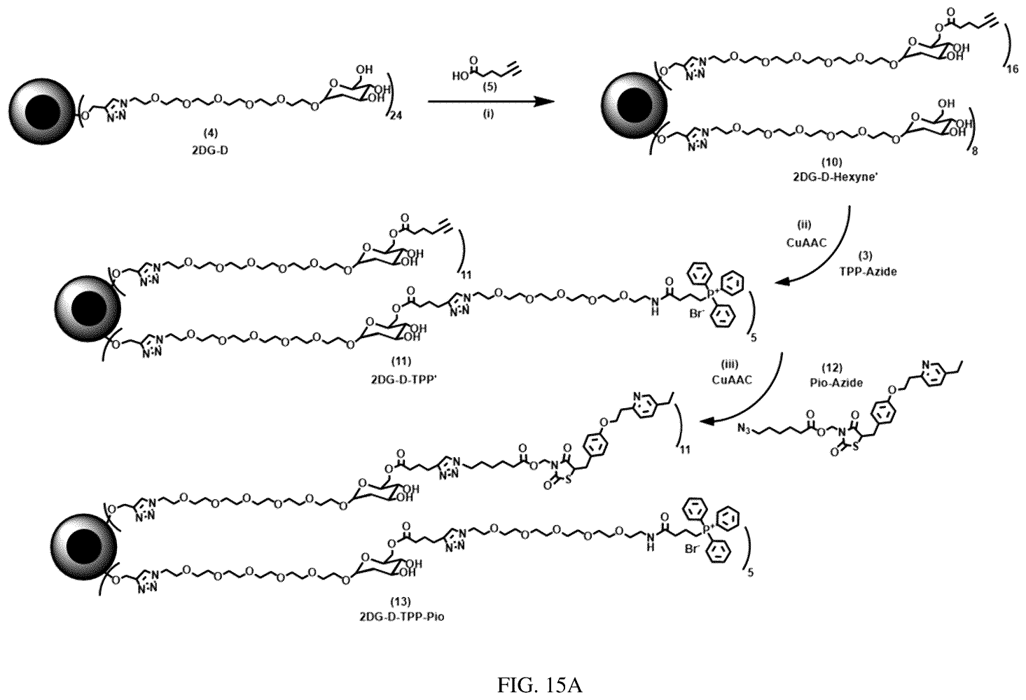

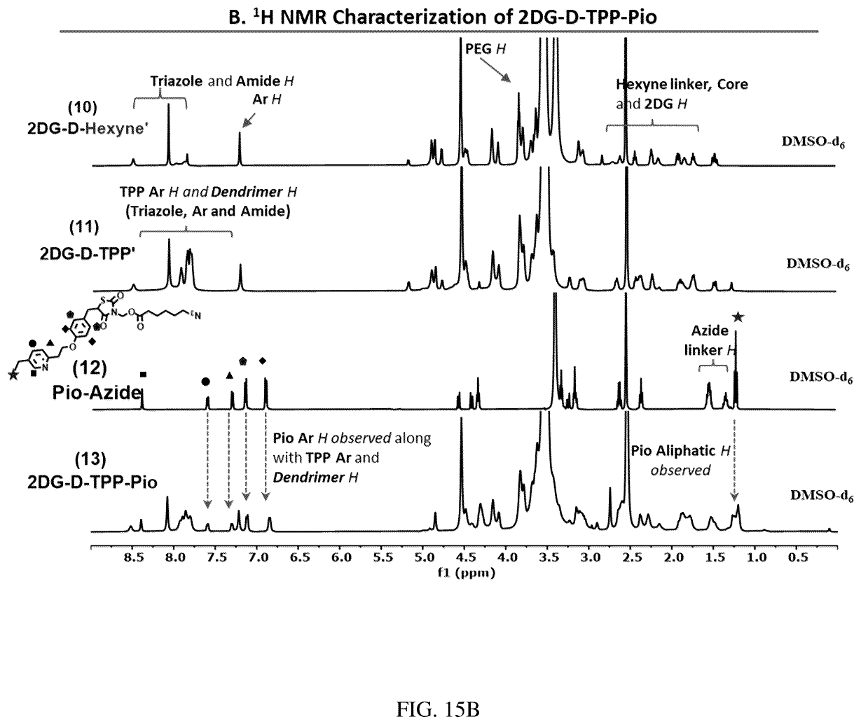

To obtain the Pio conjugate of 2DG-D-TPP for its targeted delivery to neuronal mitochondria using CuAAC reaction, Pio with a clickable azide linker was first synthesized using a previously published procedure. On the other side, ˜16 hydroxyl groups on the surface of 2DG-D (4) were modified this time, using 5-hexynoic acid (5) to afford 2DG-D-Hexyne′ (10) (FIG. 15A). The ¹H NMR showed the methylene protons from the 16 linkers in the aliphatic region of dendrimer at 81.7 ppm (32H), between 82.1 and 82.2 ppm and at 82.4 ppm (32H) (FIG. 15B). The HPLC showed a retention of 19.7 minutes with more than 99% purity. Next, we conjugated TPP-Azide (3) to the 2DG-D-Hexyne′ (10) using CuAAC reaction to synthesize the 2DG-D-TPP′ (11) with ˜11 free acetylene on its surface. The number of attached TPP molecules conjugated was calculated using the proton integration method as previously done for 2DG-D-TPP (7), suggesting the attachment of ˜5 molecules of the TPP. The HPLC purity of 2DG-D-TPP′ (11) was >97%, and the HPLC chromatogram showed a shift in the retention time from ˜19.7 minutes for 2DG-D-Hexyne (10) to ˜22.0 minutes for 2DG-D-TPP′ (11). The 2DG-D-TPP′ (11) was finally reacted with Pio-Azide (12) using CuAAC to obtain 2DG-D-TPP-Pio (13) dendrimer-drug conjugate. Conjugation of Pio was confirmed through the comparative integration of aromatic protons from the dendrimer at 87.2 ppm (16H), and the aromatic of the drug at 86.8 ppm (22H), 87.1 ppm (22H), 87.3 ppm (11H), 87.6 ppm (11H) and at 88.4 ppm (11H). The analysis of ¹H NMR confirmed the attachment of ˜11 drug molecules corresponding to ˜13 weight percent drug loading. The HPLC purity level of 2DG-D-TPP-Pio (13) was found to be more than 99%. The HPLC chromatogram showed a clear shift in the retention time from ˜22.0 to ˜23.0 min upon Pio conjugation. A key challenge in the clinical translation of nanoparticle-based therapeutics is the lack of reproducibility and scalability in their synthesis. To overcome this, we employed a streamlined and efficient synthetic strategy to develop 2DG-D-TPP-Pio (13), enabling precise characterization of intermediates at each stage, as outlined above. To assess the reproducibility of this approach, we synthesized three independent 500 mg-scale batches of the conjugate and evaluated them by ¹H NMR and HPLC. The ¹H NMR spectrum consistently showed the presence of approximately five TPP and eleven Pio molecules per dendrimer across all batches, indicating robust batch-to-batch uniformity. Correspondingly, HPLC analysis revealed identical retention times (˜23 minutes) for all three batches, confirming consistency in purity.

### 2DG-D-TTP-Cy5 was co-localized with the mitochondria of the neurons at the site of injury (FIG. 16) The in vivo study has shown that 2DG-D-TTP successfully crossed the blood brain barrier and achieved targeted delivery to the mitochondria of the neurons at the injured brain regions

### 2DG-D-TTP-Pio improved behavioral outcomes

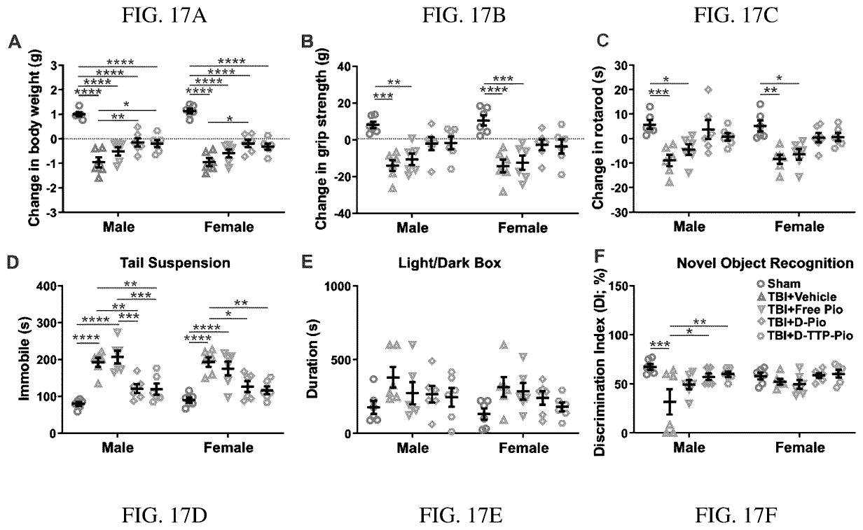

Both D-Pio and D-TTP-Pio treatments significantly improved body weight gain and decreased depression-like behaviors at 1-day post-treatment. Moreover, both D-Pio and D-TTP-Pio treatments significantly improved cognition in males. Both D-Pio and D-TTP-Pio treatments also showed a trend of improvement in muscle strength and sensorimotor coordination (FIG. 17).

### D-TTP-Pio ameliorated mitochondrial dysfunction of the neurons at the site of brain injury

D-TTP-Pio treatment significantly improved the expression of Creb1, Cycs, Aif and Pparg, compared to the TBI+vehicle and TBI+Pio (free drug) groups. Moreover, D-TTP-Pio treatment significantly increased the expression of catalase, compared to the TBI+vehicle group (FIG. 18).

### Conclusions

In this study, we successfully designed and synthesized a novel neuron-specific mitochondrial-targeted drug delivery platform, 2DG-D-TPP, utilizing a reproducible and efficient copper-catalyzed azide-alkyne cycloaddition (CuAAC) strategy. By conjugating the mitochondria-targeting moiety triphenylphosphonium (TPP) onto the 2DG-surfaced dendrimer (2DG-D) scaffold, we enabled efficient targeting of neuronal mitochondria following systemic administration in a pediatric TBI mouse model. We further functionalized the 2DG-D-TPP with Pio, creating 2DG-D-TPP-Pio conjugates as a targeted drug delivery therapy for TBI.

The results for the in vivo study have demonstrated that 2DG-D-TTP-Pio crossed the blood brain barrier and achieved targeted delivery to the mitochondria of the neurons in the site of injury, significantly improved behavioral outcomes and ameliorated the mitochondrial dysfunction. Moreover, at the same dose, 2DG-D-TTP-Pio showed a significantly higher efficacy, compared with the free drug group in improving the mitochondrial functions.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A dendrimer complex comprising
a 2-deoxyglucose (2DG) dendrimer; and
a neuroactive agent conjugated to an outer surface of the dendrimer, wherein the 2DG dendrimer is not conjugated to a prostate-specific membrane antigen (PSMA) ligand.

2. The dendrimer complex of claim 1, wherein the dendrimer is a generation 0-10 dendrimer.

3. The dendrimer complex of claim 1, wherein the dendrimer is a mixed layer dendrimer.

4. The dendrimer complex of claim 1, wherein the neuroactive agent is pioglitazone or rosiglitazone.

5. The dendrimer complex of claim 1, wherein the complex further comprises a mitochondria targeting moiety conjugated to an outer surface of the dendrimer.

6. The dendrimer complex of claim 5, wherein the mitochondria targeting moiety is selected from the group consisting of triphenylphosphonium, rhodamine derivatives, dequalinium (DQA), peptide-based targeting ligands, tetraphenylethylene (TPE) based molecule, mitochondria-penetrating peptides, indolinium based compounds, and szeto-schiller (SS) peptides.

7. The dendrimer complex of claim 1, wherein the complex further comprises one or more imaging agents and/or radioligands conjugated to an outer surface of the dendrimer.

8. A pharmaceutical composition comprising the dendrimer complex of claim 1 and a pharmaceutically acceptable carrier.

9. A method of delivering a neuroactive agent across the blood-brain barrier in a subject in need thereof, comprising administering to the subject a therapeutically effective amount of the dendrimer complex of claim 1.

10. The method of claim 9, wherein the dendrimer complex is administered via systemic administration.

11. The method of claim 9, wherein the subject has a traumatic brain injury.

12. A method of imaging neurons, comprising
contacting the neurons with the dendrimer complex of claim 1, wherein the dendrimer complex further comprises an imaging agent conjugated to an outer surface of the dendrimer; and
detecting the imaging agent.
